(19) United States
(12) Patent Application Publication
(10) Pub. No.: US 2026/0041788 A1
(43) Pub. Date: Feb. 12, 2026

(54) GENE THERAPY FOR TREATING PROPIONIC ACIDEMIA

(71) Applicant: Ultragenyx Pharmaceutical Inc., Novato, CA (US)

(72) Inventors: Matthew Scott Fuller, Millis, MA (US); Samuel Wadsworth, Monterey, MA (US); Kelly Reed Clark, Westerville, OH (US); Sean Christopher Daugherty, Petaluma, CA (US); Stewart Craig, Cambridge, MA (US)

(21) Appl. No.: 19/365,040

(22) Filed: Oct. 21, 2025

Related U.S. Application Data
(62) Division of application No. 17/278,847, now Pat. No. 12,472,268.
(60) Provisional application No. 62/739,471, filed on Oct. 1, 2018.

(51) Int. Cl. A61K 48/00; C12N 15/86

(52) U.S. Cl. CPC A61K 48/005; A61K 48/0091; C12N 15/86; C12N 2750/14143; C12N 2830/008; C12N 2830/50

(57) ABSTRACT

This present disclosure provides adeno-associated viral vectors, recombinant adeno-associated virus (rAAV), and methods of their use in gene therapy for treating propionic acidemia (PA). Also provided are pharmaceutical compositions comprising a recombinant adeno-associated virus of the invention and a pharmaceutically acceptable carrier or excipient. These pharmaceutical compositions may be useful in gene therapy for the treatment of PA caused by mutations in propionyl-CoA carboxylase α-subunit (PCCA) or mutations in propionyl-CoA carboxylase β-subunit (PCCB).

### CROSS-REFERENCE TO RELATED APPLICATIONS

This application is a divisional application of U.S. patent application Ser. No. 17/278,847, filed on Mar. 23, 2021, which is a U.S. National Stage Entry of International Application No. PCT/US2019/054003, filed on Oct. 1, 2019, which claims the benefit of and priority to U.S. Provisional Application No. 62/739,471, filed on Oct. 1, 2018, the entire disclosures of each of which are incorporated herein by reference in their entireties for all purposes.

### SEQUENCE LISTING

The instant application contains a Sequence Listing which has been submitted electronically in XML format and is hereby incorporated by reference in its entirety. Said XML copy, created on Oct. 20, 2025, is named 00021.011.1501_SL.xml and is 85,558 bytes in size.

### TECHNICAL FIELD OF THE INVENTION

The present disclosure relates generally to adeno-associated viral vectors, adeno-associated virus, and methods of their use in gene therapy for treating propionic acidemia.

### BACKGROUND OF THE INVENTION

Propionic acidemia (PA), also known as propionic aciduria, is an inborn error of organic acid metabolism caused by a deficiency of active propionyl-CoA carboxylase (PCC), an enzyme needed to convert propionyl-CoA to (D)-methylmalonyl-CoA, a key step in the catabolic pathway for odd chain fatty acids and the propiogenic amino acids, particularly isoleucine, threonine, methionine, and valine. The PCC enzyme is composed of two non-identical subunits, α and β, which are encoded by the PCCA and PCCB genes, respectively. A propionyl-CoA carboxylase deficiency can result from mutations in either PCCA or PCCB. In one study, a mutation analysis of 30 patients with PA was performed and found that 15 patients were α-subunit deficient and 15 patients were β-subunit deficient. See Yang et al., 2004, Mol Genet and Metab. 81: 335-342.

The estimated incidence of PA is 1:105,000 to 1:130,000 in the United States. This rare autosomal recessive metabolic disorder presents in the early neonatal period with poor feeding, vomiting, lethargy, seizures, and lack of muscle tone. Left untreated, death can occur quickly, due to secondary hyperammonemia, infection, cardiomyopathy, or basal ganglial stroke. PA can be diagnosed almost immediately in newborns and the disease is included in newborn screening panels in the United States.

PA is currently managed by dietary restriction of amino acid precursors, supplementation of L-carnitine to address diminished carnitine levels, and administration of antibiotics to reduce propionic acid production by intestinal bacteria. Liver transplantation is gaining a role in the management of PA in situations where the patient cannot be managed by standard treatment. However, despite the aggressive efforts to address the disease through complex combinations of nutritional, cofactor, and antibiotic therapy, the long-term prognosis for patients with PA remains poor. See van der Meer et al., 1996, Eur. J. Pediatr. 155: 205-210. Accordingly, improved therapeutic approaches are needed that address the underlying cause of the disease, namely the deficiency of PCC.

The present invention addresses this need via the creation of adeno-associated viral vectors that mediate the transfer of functional PCCA or PCCB genes to patients with PA. The present invention also describes the creation of recombinant adeno-associated virus (rAAV) that delivers functional PCCA or PCCB genes to patients with PA.

### SUMMARY OF THE INVENTION

This invention provides compositions and methods of their use in gene therapy. More specifically, provided herein are recombinant adeno-associated virus (rAAV) comprising an adeno-associated virus (AAV) capsid, and a vector genome packaged therein useful for the treatment of PA.

In one aspect, the present disclosure provides a recombinant adeno-associated virus (rAAV) comprising an AAV capsid, and a vector genome packaged therein, said vector genome comprising in 5′ to 3′ order: (a) a 5′-inverted terminal repeat sequence (5′-ITR) sequence; (b) a promoter sequence; (c) a partial or complete coding sequence for PCCA; and (d) a 3′-inverted terminal repeat sequence (3′-ITR) sequence.

In another aspect, the present disclosure provides an rAAV comprising an AAV capsid, and a vector genome packaged therein, said vector genome comprising as operably linked components in 5′ to 3′ order: (a) a 5′-ITR sequence; (b) an enhancer sequence; (c) a promoter sequence; (d) an intron sequence; (e) a partial or complete coding sequence for PCCA; (f) a polyadenylation signal sequence; and (g) a 3′-ITR sequence.

In certain embodiments, the present disclosure provides an rAAV comprising an AAV capsid, and a vector genome packaged therein that comprises an AAV 5′-ITR sequence, a promoter sequence, a partial or complete coding sequence for PCCA or an isoform thereof, or a functional fragment or functional variant thereof, and an AAV 3′-ITR sequence.

In certain embodiments, the present disclosure provides an rAAV comprising an AAV capsid, and a vector genome packaged therein that comprises an AAV 5′-ITR, a promoter sequence, a truncated or complete nucleotide sequence of human PCCA 5′-untranslated region (5′-UTR), a partial or complete coding sequence for PCCA or an isoform thereof, or a functional fragment or functional variant thereof, a truncated or complete human PCCA 3′-untranslated region (3′-UTR) nucleotide sequence, and an AAV 3′-ITR.

In certain embodiments, the present disclosure provides an rAAV comprising an AAV capsid, and a vector genome packaged therein that comprises complete human PCCA 5′-UTR represented by SEQ ID NO: 30. In certain embodiments, the packaged genome comprises truncated human PCCA 5′-UTR nucleotide sequence. In some embodiments, the truncated nucleotide sequence of human PCCA 5′-UTR comprises a portion of SEQ ID NO: 30. In some embodiments, the truncated nucleotide sequence of human PCCA 5′-UTR comprises a nucleotide sequence of at least 100 continuous nucleotides, which is at least 95% identical to an equal length region of SEQ ID NO: 30. In certain embodiments, the truncated or complete human PCCA 5′-UTR is located between the intron sequence and the partial or complete coding sequence for PCCA when the partial or complete coding sequence in the packaged vector genome is for PCCA.

In certain embodiments, the packaged vector genome comprises complete human PCCA 3′-UTR represented by SEQ ID NO: 31. In certain embodiments, the packaged genome comprises truncated human PCCA 3′-UTR nucleotide sequence. In some embodiments, the truncated nucleotide sequence of human PCCA 3′-UTR comprises a portion of SEQ ID NO: 31. In some embodiments, the truncated nucleotide sequence of human PCCA 3′-UTR comprises a nucleotide sequence of at least 15 continuous nucleotides, which is at least 95% identical to an equal length region of SEQ ID NO: 31. In certain embodiments, the truncated or complete human PCCA 3′-UTR is located between the polyadenylation signal sequence; and the 3′-ITR sequence when the partial or complete coding sequence in the packaged vector genome is for PCCA.

In one embodiment, the partial or complete coding sequence for PCCA is a wild-type coding sequence. In an alternative embodiment, the partial or complete coding sequence for PCCA is a codon-optimized coding sequence. In one exemplary embodiment, the partial or complete coding sequence for PCCA is codon-optimized for expression in humans.

In some embodiments, PCCA is encoded by the wild-type coding sequence shown in SEQ ID NO: 1. In another embodiment, a coding sequence expressing a natural isoform or variant of PCCA may be used, such as those shown in UniProtKB/Swiss-Prot Accession Nos. P05165-1 (SEQ ID NO: 25), P05165-2 (SEQ ID NO: 26), and P05165-3 (SEQ ID NO: 27). In certain embodiments, PCCA is encoded by a codon-optimized coding sequence. In some embodiments, PCCA is encoded by a codon-optimized coding sequence that is less than 80% identical to the wild-type coding sequence shown in SEQ ID NO: 1. In some exemplary embodiments, PCCA is encoded by a codon-optimized coding sequence selected from SEQ ID NOs: 2-6. In some embodiments, PCCA is encoded by a codon-optimized coding sequence which is at least 80% identical to a sequence selected from SEQ ID NOs: 2-6. In some embodiments, PCCA is encoded by a codon-optimized coding sequence which is at least 90% identical to a sequence selected from SEQ ID NOs: 2-6. In some embodiments, PCCA is encoded by a codon-optimized coding sequence which is at least 95% identical to a sequence selected from SEQ ID NOs: 2-6. In some embodiments, the coding sequence for PCCA may further comprise a stop codon (TGA, TAA, or TAG) at the 3′ end. In some embodiments, the expressed PCCA comprises or consists of an amino acid sequence of SEQ ID NO: 16.

In another aspect, the present disclosure provides a recombinant adeno-associated virus (rAAV) comprising an AAV capsid, and a vector genome packaged therein, said vector genome comprising in 5′ to 3′ order: (a) a 5′-ITR sequence; (b) a promoter sequence; (c) a partial or complete coding sequence for PCCB; and (d) a 3′-ITR sequence.

In another aspect, the present disclosure provides an rAAV comprising an AAV capsid, and a vector genome packaged therein, said vector genome comprising as operably linked components in 5′ to 3′ order: (a) a 5′-ITR sequence; (b) an enhancer sequence; (c) a promoter sequence; (d) an intron sequence; (e) a partial or complete coding sequence for PCCB; (f) a polyadenylation signal sequence; and (g) a 3′-ITR sequence.

In certain embodiments, the present disclosure provides an rAAV comprising an AAV capsid, and a vector genome packaged therein that comprises an AAV 5′-ITR sequence, a promoter sequence, a partial or complete coding sequence for PCCB or an isoform thereof, or a functional fragment or functional variant thereof, and an AAV 3′-ITR sequence.

In certain embodiments, the present disclosure provides an rAAV comprising an AAV capsid, and a vector genome packaged therein that comprises an AAV 5′-ITR, a promoter sequence, a truncated or complete nucleotide sequence of human PCCB 5′-untranslated region (5′-UTR), a partial or complete coding sequence for PCCB or an isoform thereof, or a functional fragment or functional variant thereof, a truncated or complete human PCCB 3′-untranslated region (3′-UTR) nucleotide sequence, and an AAV 3′-ITR.

In certain embodiments, the packaged vector genome comprises complete human PCCB 5′-UTR represented by SEQ ID NO: 32. In certain embodiments, the packaged vector genome comprises truncated human PCCB 5′-UTR nucleotide sequence. In some embodiments, the truncated nucleotide sequence of human PCCB 5′-UTR comprises a portion of SEQ ID NO: 32. In some embodiments, the truncated nucleotide sequence of human PCCB 5′-UTR comprises a nucleotide sequence of at least 100 continuous nucleotides, which is at least 95% identical to an equal length region of SEQ ID NO: 32. In certain embodiments, the truncated or complete human PCCB 5′-UTR is located between the intron sequence and the partial or complete coding sequence for PCCB when the partial or complete coding sequence in the packaged vector genome is for PCCB.

In certain embodiments, the packaged vector genome comprises complete human PCCB 3′-UTR represented by SEQ ID NO: 33. In certain embodiments, the packaged vector genome comprises truncated human PCCB 3′-UTR nucleotide sequence. In some embodiments, the truncated nucleotide sequence of human PCCB 3′-UTR comprises a portion of SEQ ID NO: 33. In some embodiments, the truncated nucleotide sequence of human PCCB 3′-UTR comprises a nucleotide sequence of at least 15 continuous nucleotides, which is at least 95% identical to an equal length region of SEQ ID NO: 33. In certain embodiments, the truncated or complete human PCCB 3′-UTR is located between the polyadenylation signal sequence; and the 3′-ITR sequence when the partial or complete coding sequence in the packaged vector genome is for PCCB.

In one embodiment, the partial or complete coding sequence for PCCB is a wild-type coding sequence. In an alternative embodiment, the partial or complete coding sequence for PCCB is a codon-optimized coding sequence. In one exemplary embodiment, the partial or complete coding sequence for PCCB is codon-optimized for expression in humans.

In some embodiments, PCCB is encoded by the wild-type coding sequence shown in SEQ ID NO: 7. In another embodiment, a coding sequence expressing a natural isoform or variant of PCCB may be used, such as those shown in UniProtKB/Swiss-Prot Accession Nos. P05166-1 (SEQ ID NO: 28) and P05166-2 (SEQ ID NO: 29). In alternative embodiments, PCCB is encoded by a codon-optimized coding sequence. In some embodiments, PCCB is encoded by a codon-optimized coding sequence that is less than 80% identical to the wild-type coding sequence shown in SEQ ID NO: 7. In some exemplary embodiments, PCCB is encoded by a codon-optimized coding sequence selected from SEQ ID NOs: 8-12. In some embodiments, PCCB is encoded by a codon-optimized coding sequence which is at least 80% identical to a sequence selected from SEQ ID NOs: 8-12. In some embodiments, PCCB is encoded by a codon-optimized coding sequence which is at least 90% identical to a sequence selected from SEQ ID NOs: 8-12. In some embodiments, PCCB is encoded by a codon-optimized coding sequence which is at least 95% identical to a sequence selected from SEQ ID NOs: 8-12. In some embodiments, the coding sequence for PCCB may further comprise a stop codon (TGA, TAA, or TAG) at the 3′ end. In some embodiments, the expressed PCCB comprises or consists of an amino acid sequence of SEQ ID NO: 17.

In some embodiments, the 5′-ITR sequence is from AAV2. In some embodiments, the 3′-ITR sequence is from AAV2. In some embodiments, the 5′-ITR sequence and the 3′-ITR sequence are from AAV2. In some embodiments, the 5′-ITR sequence and/or the 3′-ITR sequence comprise or consist of SEQ ID NO: 15. In other embodiments, the 5′-ITR sequence and/or the 3′-ITR sequence are from a non-AAV2 source.

In one embodiment, the promoter is selected from a chicken 3-actin (CBA) promoter, a cytomegalovirus (CMV) immediate early gene promoter, a transthyretin (TTR) promoter, a thyroxine binding globulin (TBG) promoter, an alpha-1 anti-trypsin (A1AT) promoter, a CAG promoter (constructed using the CMV early enhancer element, the promoter, the first exon, and the first intron of CBA gene, and the splice acceptor of the rabbit beta-globin gene), a PCCA gene-specific endogenous promoter, and a PCCB gene-specific endogenous promoter. In an exemplary embodiment, the promoter is the CBA promoter. In one embodiment, the CBA promoter comprises or consists of SEQ ID NO: 18. In some embodiments, the promoter is a gene-specific endogenous promoter. In one some embodiment, the promoter comprises native gene promoter elements. In an exemplary embodiment, the promoter is the PCCA gene-specific endogenous promoter comprising a nucleotide sequence of at least 15 continuous nucleotides, which is at least 95% identical to an equal length region of SEQ ID NO: 34 when the partial or complete coding sequence in the vector genome is for PCCA. In an exemplary embodiment, the promoter is the PCCB gene-specific endogenous promoter comprising a nucleotide sequence of at least 15 continuous nucleotides, which is at least 95% identical to an equal length region of SEQ ID NO: 36 when the partial or complete coding sequence in the vector genome is for PCCB.

In some embodiments, the packaged vector genome further comprises one or more enhancer sequences. In one embodiment, the enhancer is selected from a cytomegalovirus (CMV) immediate early gene enhancer, a transthyretin enhancer (enTTR), a chicken β-actin (CBA) enhancer, an En34 enhancer, and an apolipoprotein E (ApoE) enhancer. In an exemplary embodiment, the enhancer is the CMV enhancer. In one embodiment, the CMV enhancer comprises or consists of SEQ ID NO: 19. In certain embodiments, the enhancer is located upstream of the promoter sequence.

In some embodiments, packaged vector genome further comprises one or more intron sequences. In one embodiment, the intron is selected from an SV40 Small T intron, a rabbit hemoglobin subunit beta (rHBB) intron, a human beta globin IVS2 intron, a β-globin/IgG chimeric intron (Promega chimeric intron), or an hFIX intron. In one exemplary embodiment, the intron is the SV40 Small T intron. In one embodiment, the SV40 Small T intron sequence comprises or consists of SEQ ID NO: 20. In another exemplary embodiment, the intron is the rHBB intron. In one embodiment, the rHBB intron sequence comprises or consists of SEQ ID NO: 21.

In some embodiments, packaged vector genome further comprises a consensus Kozak sequence. In some embodiments, the consensus Kozak sequence is located downstream of an intron sequence. In one embodiment, the consensus Kozak sequence is GCCGCC (SEQ ID NO: 24). In certain embodiments, the consensus Kozak sequence is located upstream of coding sequence for PCCA. In certain embodiments, the consensus Kozak sequence is located upstream of coding sequence for PCCB.

In some embodiments, packaged vector genome further comprises a polyadenylation signal sequence. In certain embodiments, the polyadenylation signal sequence is selected from a bovine growth hormone (BGH) polyadenylation signal sequence, an SV40 polyadenylation signal sequence, a rabbit beta globin polyadenylation signal sequence, a PCCA gene-specific endogenous polyadenylation signal sequence, a PCCB gene-specific endogenous polyadenylation signal sequence. In an exemplary embodiment, the polyadenylation signal sequence is the bovine growth hormone (BGH) polyadenylation signal sequence. In one embodiment, the BGH polyadenylation signal sequence comprises or consists of SEQ ID NO: 22. In another exemplary embodiment, the polyadenylation signal sequence is the SV40 polyadenylation signal sequence. In one embodiment, the SV40 polyadenylation signal sequence comprises or consists of SEQ ID NO: 23. In another exemplary embodiment, the polyadenylation signal sequence comprises the PCCA gene-specific endogenous polyadenylation signal sequence when the partial or complete coding sequence in the vector genome is for PCCA. In one embodiment, the PCCA gene-specific endogenous polyadenylation signal sequence comprises a nucleotide sequence of at least 15 continuous nucleotides, which is at least 95% identical to an equal length region of SEQ ID NO: 35. In another exemplary embodiment, the polyadenylation signal sequence comprises the PCCB gene-specific endogenous polyadenylation signal sequence when the partial or complete coding sequence in the vector genome is for PCCB. In one embodiment, the PCCB gene-specific endogenous polyadenylation signal sequence comprises a nucleotide sequence of at least 15 continuous nucleotides, which is at least 95% identical to an equal length region of SEQ ID NO: 37.

In certain embodiments, the AAV capsid is from an AAV of serotype 1, 2, 3, 4, 5, 6, 7, 8, 9, 10, 11, 12, rh10, or hu37. In one exemplary embodiment, the AAV capsid is from AAV9. In another exemplary embodiment, the AAV capsid is from AAV8. In another exemplary embodiment, the AAV capsid is an AAV9 variant capsid.

In certain embodiments, the present disclosure provides rAAV in which the coding sequence is for PCCA and not for PCCB, the promoter is a PCCA gene-specific endogenous promoter and not a PCCB gene-specific endogenous promoter, and the polyadenylation signal sequence is a PCCA gene-specific endogenous polyadenylation signal sequence and not a PCCB gene-specific endogenous polyadenylation signal sequence. In certain embodiments, the present disclosure provides rAAV in which the coding sequence is for PCCB and not for PCCA, the promoter is a PCCB gene-specific endogenous promoter and not a PCCA gene-specific endogenous promoter, and the polyadenylation signal sequence is a PCCB gene-specific endogenous polyadenylation signal sequence and not a PCCA gene-specific endogenous polyadenylation signal sequence.

In some aspects, the present disclosure provides novel codon-optimized nucleic acid sequences encoding PCCA. In one embodiment, the codon-optimized nucleic acid sequence encoding PCCA is less than 80% identical to the wild-type coding sequence shown in SEQ ID NO: 1. In some embodiments, the codon-optimized nucleic acid sequence encoding PCCA is at least 80%, 85%, 90%, 91%, 92%, 93%, 94%, 95%, 96%, 97%, 98%, 99% or more identical to SEQ ID NOs: 2-6. In some embodiments, the present disclosure provides nucleic acid sequences which are less than 80% identical to the wild-type coding sequence shown in SEQ ID NO: 1 and are at least 80%, 85%, 90%, 91%, 92%, 93%, 94%, 95%, 96%, 97%, 98%, 99% or more identical to SEQ ID NOs: 2-6. In exemplary embodiments, the present disclosure provides a nucleic acid sequence encoding PCCA selected from SEQ ID NOs: 2-6. Further provided are fragments of the nucleic acid sequences shown in SEQ ID NOs: 2-6 which encode a polypeptide having functional PCCA activity. In some embodiments, the nucleic acid sequence encoding PCCA may further comprise a stop codon (TGA, TAA, or TAG) at the 3′ end.

In some aspects, the present disclosure provides novel codon-optimized nucleic acid sequences encoding PCCB. In one embodiment, the codon-optimized nucleic acid sequence encoding PCCB is less than 80% identical to the wild-type coding sequence shown in SEQ ID NO: 7. In some embodiments, the codon-optimized nucleic acid sequence encoding PCCB is at least 80%, 85%, 90%, 91%, 92%, 93%, 94%, 95%, 96%, 97%, 98%, 99% or more identical to SEQ ID NOs: 8-12. In some embodiments, the present disclosure provides nucleic acid sequences which are less than 80% identical to the wild-type coding sequence shown in SEQ ID NO: 7 and are at least 80%, 85%, 90%, 91%, 92%, 93%, 94%, 95%, 96%, 97%, 98%, 99% or more identical to SEQ ID NOs: 8-12. In exemplary embodiments, the present disclosure provides a nucleic acid sequence encoding PCCB selected from SEQ ID NOs: 8-12. Further provided are fragments of the nucleic acid sequences shown in SEQ ID NOs: 8-12, which encode a polypeptide having functional PCCB activity. In some embodiments, the nucleic acid sequence encoding PCCB may further comprise a stop codon (TGA, TAA, or TAG) at the 3′ end.

In certain embodiments, the present disclosure provides recombinant adeno-associated virus (rAAV) useful as agents for gene therapy in the treatment of PA, wherein said rAAV comprises an AAV capsid, and a vector genome as described herein packaged therein. In some embodiments, the AAV capsid is from an AAV of serotype 1, 2, 3, 4, 5, 6, 7, 8, 9, 10, 11, 12, rh10, or hu37 (i.e., AAV1, AAV2, AAV3, AAV4, AAV5, AAV6, AAV7, AAV9, AAV10, AAV11, AAV12, AAVrh10, or AAVhu37). In an exemplary embodiment, the AAV vector is an AAV serotype 9 (AAV9) vector, an AAV9 variant vector, an AAV serotype 8 (AAV8) vector, or an AAV serotype 2 (AAV2) vector.

In certain embodiments, the present disclosure provides an rAAV useful for the treatment of propionic acidemia (PA), said rAAV comprising an AAV capsid, and a vector genome packaged therein, said vector genome comprising as operably linked components in 5′ to 3′ order: (a) a 5′-ITR sequence; (b) an enhancer sequence; (c) a promoter sequence; (d) an intron sequence; (e) a coding sequence for PCCA selected from SEQ ID NOs: 1-6; (f) a polyadenylation signal sequence; and (g) a 3′ITR sequence.

In certain embodiments, the present disclosure provides an rAAV useful for the treatment of propionic acidemia (PA), said rAAV comprising an AAV capsid, and a vector genome packaged therein, said vector genome comprising as operably linked components in 5′ to 3′ order: (a) a 5′-ITR sequence; (b) an enhancer sequence; (c) a promoter sequence; (d) an intron sequence; (e) a coding sequence for PCCB selected from SEQ ID NOs: 7-12; (f) a polyadenylation signal sequence; and (g) a 3′ITR sequence.

In certain embodiments, the present disclosure provides an rAAV useful for the treatment of propionic acidemia (PA), said rAAV comprising an AAV9 capsid, and a vector genome packaged therein, said vector genome comprising as operably linked components in 5′ to 3′ order: (a) a 5′-ITR sequence; (b) an enhancer sequence; (c) a promoter sequence; (d) an intron sequence; (e) a coding sequence for PCCA selected from SEQ ID NOs: 1-6; (f) a polyadenylation signal sequence; and (g) a 3′-ITR sequence.

In certain embodiments, the present disclosure provides an rAAV useful for the treatment of propionic acidemia (PA), said rAAV comprising an AAV9 capsid, and a vector genome packaged therein, said vector genome comprising as operably linked components in 5′ to 3′ order: (a) a 5′-ITR sequence; (b) an enhancer sequence; (c) a promoter sequence; (d) an intron sequence; (e) a coding sequence for PCCB selected from SEQ ID NOs: 7-12; (f) a polyadenylation signal sequence; and (g) a 3′-ITR sequence.

In certain embodiments, the present disclosure provides an rAAV useful for the treatment of propionic acidemia (PA), said rAAV comprising an AAV9 capsid, and a vector genome packaged therein, said vector genome comprising as operably linked components in 5′ to 3′ order: (a) an AAV2 5′-ITR sequence; (b) a CMV enhancer sequence; (c) a CBA promoter sequence; (d) an rHBB or an SV40 Small T intron sequence; (e) a coding sequence for PCCA selected from SEQ ID NOs: 1-6; (f) a BGH or SV40 polyadenylation signal sequence; and (g) an AAV2 3′-ITR sequence.

In certain embodiments, the present disclosure provides an rAAV useful for the treatment of propionic acidemia (PA), said rAAV comprising an AAV9 capsid, and a vector genome packaged therein, said vector genome comprising as operably linked components in 5′ to 3′ order: (a) an AAV2 5′-ITR sequence; (b) a CMV enhancer sequence; (c) a CBA promoter sequence; (d) an rHBB or an SV40 Small T intron sequence; (e) a coding sequence for PCCB selected from SEQ ID NOs: 7-12; (f) a BGH or SV40 polyadenylation signal sequence; and (g) an AAV2 3′-ITR sequence.

In certain embodiments, the present disclosure provides a recombinant nucleic acid construct comprising (a) a 5′-ITR sequence; (b) a promoter sequence; (c) a partial or complete coding sequence for PCCA; and (d) a 3′-ITR sequence. In certain embodiments, the present disclosure provides a recombinant nucleic acid construct comprising (a) a 5′-ITR sequence; (b) a promoter sequence; (c) a partial or complete coding sequence for PCCB; and (d) a 3′-ITR sequence.

In some aspects, the present disclosure provides the use of an rAAV disclosed herein for the treatment of PA, wherein the rAAV includes an AAV capsid and a vector genome packaged therein. In some embodiments, the rAAV contains a packaged genome comprising as operably linked components in 5′ to 3′ order: a 5′-ITR, a promoter sequence, a partial or complete coding sequence for PCCA or an isoform thereof, or a functional fragment or functional variant thereof, and a 3′-ITR. In an exemplary embodiment, the packaged genome also comprises an enhancer sequence upstream of the promoter sequence, an intron downstream of the promoter, and a polyadenylation sequence upstream of the 3′-ITR. In one exemplary embodiment, the rAAV contains a packaged genome comprising as operably linked components in 5′ to 3′ order: an AAV2 5′-ITR sequence, a CMV enhancer, a CBA promoter, an SV40 Small T intron, a coding sequence for PCCA, a BGH polyadenylation signal sequence, and an AAV2 3′-ITR. In some embodiments, the packaged genome further comprises a consensus Kozak sequence located downstream of the intron sequence. In some embodiments, the coding sequence for PCCA is selected from SEQ ID NOs: 1-6. In some embodiments, the capsid is an AAV9 capsid.

In some aspects, the present disclosure provides the use of an rAAV disclosed herein for the treatment of PA, wherein the rAAV includes an AAV capsid and a vector genome packaged therein. In some embodiments, the packaged genome comprising as operably linked components in 5′ to 3′ order comprises: a 5′-ITR, a promoter sequence (e.g., PCCA gene-specific endogenous promoter sequence), a truncated or complete nucleotide sequence of human PCCA 5′-UTR, a partial or complete coding sequence for PCCA or an isoform thereof, or a functional fragment or functional variant thereof, a truncated or complete nucleotide sequence of human PCCA 3′-UTR, and a 3′-ITR. In an exemplary embodiment, the packaged genome also comprises an enhancer sequence upstream of the promoter sequence, an intron downstream of the promoter, and a PCCA gene-specific polyadenylation sequence upstream of the 3′-ITR. In some embodiments, the packaged genome further comprises a consensus Kozak sequence located downstream of the truncated or complete nucleotide sequence of human PCCA 5′-UTR and upstream of partial or complete coding sequence for PCCA or an isoform thereof, or a functional fragment or functional variant thereof. In some embodiments, the coding sequence for PCCA is selected from SEQ ID NOs: 1-6. In some embodiments, the capsid is an AAV9 capsid.

In some aspects, the present disclosure provides the use of an rAAV disclosed herein for the treatment of PA, wherein the rAAV includes an AAV capsid and a vector genome packaged therein. In some embodiments, the packaged genome comprising as operably linked components in 5′ to 3′ order comprises: a 5′-ITR, a promoter sequence, a partial or complete coding sequence for PCCB or an isoform thereof, or a functional fragment or functional variant thereof, and a 3′-ITR. In an exemplary embodiment, the packaged genome also comprises an enhancer sequence upstream of the promoter sequence, an intron downstream of the promoter, and a polyadenylation sequence upstream of the 3′-ITR. In one exemplary embodiment, the packaged genome comprising as operably linked components in 5′ to 3′ order comprises: an AAV2 5′-ITR sequence, a CMV enhancer, a CBA promoter, an SV40 Small T intron, a coding sequence for PCCB, a BGH polyadenylation signal sequence, and an AAV2 3′-ITR. In some embodiments, the packaged genome further comprises a consensus Kozak sequence located downstream of the intron sequence. In some embodiments, the coding sequence for PCCB is selected from SEQ ID NOs: 7-12. In some embodiments, the capsid is an AAV9 capsid.

In some aspects, the present disclosure provides the use of an rAAV disclosed herein for the treatment of PA, wherein the rAAV includes an AAV capsid and a vector genome packaged therein. In some embodiments, the packaged genome comprising as operably linked components in 5′ to 3′ order comprises: a 5′-ITR, a promoter sequence (e.g., PCCB gene-specific endogenous promoter sequence), a truncated or complete nucleotide sequence of human PCCB 5′-UTR, a partial or complete coding sequence for PCCB or an isoform thereof, or a functional fragment or functional variant thereof, a truncated or complete nucleotide sequence of human PCCB 3′-UTR, and a 3′-ITR. In an exemplary embodiment, the packaged genome also comprises an enhancer sequence upstream of the promoter sequence, an intron downstream of the promoter, and a PCCB gene-specific polyadenylation sequence upstream of the 3′-ITR. In some embodiments, the packaged genome further comprises a consensus Kozak sequence located downstream of the truncated or complete nucleotide sequence of human PCCB 5′-UTR and upstream of partial or complete coding sequence for PCCB or an isoform thereof, or a functional fragment or functional variant thereof. In some embodiments, the coding sequence for PCCB is selected from SEQ ID NOs: 7-12. In some embodiments, the capsid is an AAV9 capsid.

The present disclosure further relates to pharmaceutical compositions comprising an rAAV disclosed herein. In some embodiments, the pharmaceutical composition comprises a pharmaceutically acceptable carrier or excipient. In some embodiments, the pharmaceutical composition is formulated for subcutaneous, intramuscular, intradermal, intraperitoneal, or intravenous administration. In an exemplary embodiment, the pharmaceutical composition is formulated for intravenous administration.

In yet another aspect, the present disclosure provides methods of treating PA in a human subject comprising administering to the human subject a therapeutically effective amount of at least one rAAV disclosed herein. In one embodiment, the present disclosure provides a method of treating PA comprising administering an rAAV that includes an AAV capsid and a vector genome packaged therein, wherein the vector genome comprises a partial or complete coding sequence for PCCA or an isoform thereof, or a functional fragment or functional variant thereof. In another embodiment, the present disclosure provides a method of treating PA comprising administering an rAAV that includes an AAV capsid and a vector genome packaged therein, wherein the vector genome comprises a partial or complete coding sequence for PCCB or an isoform thereof, or a functional fragment or functional variant thereof.

In certain embodiments, the present disclosure provides a method of treating PA comprising administering (1) an rAAV that includes an AAV capsid and a vector genome packaged therein, wherein the vector genome comprises a partial or complete coding sequence for PCCA or an isoform thereof, or a functional fragment or functional variant thereof; and (2) an rAAV that includes an AAV capsid and a vector genome packaged therein, wherein the vector genome comprises a partial or complete coding sequence for PCCB or an isoform thereof, or a functional fragment or functional variant thereof. In some embodiments, the rAAV of (1) and (2) may administered simultaneously. In some embodiments, the rAAV of (1) and (2) may administered sequentially. In some embodiments, the rAAV of (1) and (2) may administered separately.

In certain embodiments, the present disclosure provides a method of treating PA comprising administering (1) an rAAV that includes an AAV capsid and a vector genome packaged therein in which the coding sequence is for PCCA and not for PCCB, the promoter is a PCCA gene-specific endogenous promoter and not a PCCB gene-specific endogenous promoter, and the polyadenylation signal sequence is a PCCA gene-specific endogenous polyadenylation signal sequence and not a PCCB gene-specific endogenous polyadenylation signal sequence; and/or; and (2) an rAAV that includes an AAV capsid and a vector genome in which the coding sequence is for PCCB and not for PCCA, the promoter is a PCCB gene-specific endogenous promoter and not a PCCA gene-specific endogenous promoter, and the polyadenylation signal sequence is a PCCB gene-specific endogenous polyadenylation signal sequence and not a PCCA gene-specific endogenous polyadenylation signal sequence; and/or. In some embodiments, the rAAV of (1) and (2) may administered simultaneously. In some embodiments, the rAAV of (1) and (2) may administered sequentially. In some embodiments, the rAAV of (1) and (2) may administered separately.

In certain embodiments, the present disclosure provides methods of treating PA in a human subject comprising administering to a human subject diagnosed with at least one mutation in PCCA a therapeutically effective amount of at least one rAAV disclosed herein. In one embodiment, the present disclosure provides a method of treating PA in a human subject diagnosed with at least one mutation in PCCA comprising administering an rAAV that includes an AAV capsid and a vector genome packaged therein, wherein the vector genome comprises a partial or complete coding sequence for PCCA or an isoform thereof, or a functional fragment or functional variant thereof. In certain embodiments, the present disclosure provides a method of treating PA in a human subject diagnosed with at least one mutation in PCCA comprising administering an rAAV that includes an AAV capsid and a vector genome packaged therein in which the coding sequence is for PCCA and not for PCCB, the promoter is a PCCA gene-specific endogenous promoter and not a PCCB gene-specific endogenous promoter, and the polyadenylation signal sequence is a PCCA gene-specific endogenous polyadenylation signal sequence and not a PCCB gene-specific endogenous polyadenylation signal sequence. In certain embodiments, the mutation in PCCA is selected from Table 1. In some embodiments, the coding sequence for PCCA is selected from SEQ ID NOs: 1-6. In some embodiments, the capsid is an AAV9 capsid.

In some aspects, the present disclosure provides methods of treating PA in a human subject comprising administering to a human subject diagnosed with at least one mutation in PCCB a therapeutically effective amount of at least one rAAV disclosed herein. In one embodiment, the present disclosure provides a method of treating PA in a human subject diagnosed with at least one mutation in PCCB comprising administering an rAAV that includes an AAV capsid and a vector genome packaged therein, wherein the vector genome comprises a partial or complete coding sequence for PCCB or an isoform thereof, or a functional fragment or functional variant thereof. In certain embodiments, the present disclosure provides a method of treating PA in a human subject diagnosed with at least one mutation in PCCB comprising administering an rAAV that includes an AAV capsid and a vector genome packaged therein in which the coding sequence is for PCCB and not for PCCA, the promoter is a PCCB gene-specific endogenous promoter and not a PCCA gene-specific endogenous promoter, and the polyadenylation signal sequence is a PCCB gene-specific endogenous polyadenylation signal sequence and not a PCCA gene-specific endogenous polyadenylation signal sequence. In certain embodiments, the mutation in PCCB is selected from Table 2. In some embodiments, the coding sequence for PCCB is selected from SEQ ID NOs: 7-12. In some embodiments, the capsid is an AAV9 capsid.

In some embodiments, the rAAV is administered subcutaneously, intramuscularly, intradermally, intraperitoneally, or intravenously. In an exemplary embodiment, the rAAV is administered intravenously. In some embodiments, the rAAV is administered at a dose of about 1×10¹¹ to about 1×10¹⁴ genome copies (GC)/kg. In further embodiments, the rAAV is administered at a dose of about 1×10¹² to about 1×10¹³ genome copies (GC)/kg. In some embodiments, a single dose of rAAV is administered. In other embodiments, multiple doses of rAAV are administered.

In some aspects, provided herein are host cells comprising a recombinant nucleic acid molecule, an AAV vector, or an rAAV disclosed herein. In specific embodiments, the host cells may be suitable for the propagation of AAV. In certain embodiments, the host cell is selected from a HeLa, Cos-7, HEK293, A549, BHK, Vero, RD, HT-1080, ARPE-19, or MRC-5 cell.

These and other aspects and features of the invention are described in the following sections of the present disclosure.

### BRIEF DESCRIPTION OF THE DRAWINGS

The invention can be more completely understood with reference to the following drawings.

FIG. 1 is an illustrative diagram showing an exemplary packaged vector genome construct comprising PCCA, according to one embodiment. 5′-ITR, CMV enhancer, chicken (3-actin promoter, SV40 small T intron, consensus Kozak sequence, PCCA coding sequence, SV40 polyadenylation signal, and 3′-ITR are represented by 1, 2, 3, 4, 5, 6, 7, and 8, respectively in the diagram.

FIG. 2 is an illustrative diagram showing an exemplary packaged vector genome construct comprising PCCB, according to one embodiment. 5′-ITR, CMV enhancer, chicken (3-actin promoter, SV40 small T intron, consensus Kozak sequence, PCCB coding sequence, SV40 polyadenylation signal, and 3′-ITR are represented by 1, 2, 3, 4, 5, 6, 7, and 8, respectively in the diagram.

FIG. 3 is a bar graph showing fold-change in PCCA RNA expression as determined by RT-qPCR following transfection of a continuous hepatocyte cell line (HepG2) with a control plasmid (plasmid with an empty vector, no TX), an rAAV vector plasmid carrying a nucleotide sequence encoding enhanced green fluorescent protein, eGFP (DTC343), an rAAV vector plasmid carrying a nucleotide sequence encoding the complete native sequence of human PCCA (DTC346), or an rAAV vector plasmid carrying a nucleotide sequence encoding a codon-optimized sequence of human PCCA (DTC347, DTC348, or DTC349).

FIG. 4A is an image showing protein expression levels, detected by Western blot, in continuous hepatocyte cell line (HepG2) transfected with a control plasmid (plasmid with an empty vector, no TX), an rAAV vector plasmid carrying a nucleotide sequence encoding the complete native sequence of human PCCA (DTC346), an rAAV vector plasmid carrying a nucleotide sequence encoding a codon-optimized sequence of human PCCA (DTC347, DTC348, or DTC349), an rAAV vector plasmid carrying a nucleotide sequence encoding the complete native sequence of human PCCA and a nucleotide sequence encoding the complete native sequence of human PCCB (DTC365), or an rAAV vector plasmid carrying a nucleotide sequence encoding enhanced green fluorescent protein, eGFP (DTC343), from lane 1 to lane 7, respectively. β-actin (“Actin”) was used as a loading control.

FIG. 4B is an image showing protein expression levels, detected by Western blot, in continuous hepatocyte cell line (Huh7) transfected with a control plasmid (plasmid with an empty vector, no TX), an rAAV vector plasmid carrying a nucleotide sequence encoding enhanced green fluorescent protein, eGFP (DTC343), an rAAV vector plasmid carrying a nucleotide sequence encoding the complete native sequence of human PCCA (DTC346), an rAAV vector plasmid carrying a nucleotide sequence encoding a codon-optimized sequence of human PCCA (DTC347, DTC348, or DTC349), or an rAAV vector plasmid carrying a nucleotide sequence encoding the complete native sequence of human PCCA and a nucleotide sequence encoding the complete native sequence of human PCCB (DTC365), from lane 1 to lane 7, respectively. β-actin (“Actin”) was used as a loading control.

FIG. 5 is an image showing PCCA protein expression levels, detected by Western blot, in whole cell lysates (WCL); subcellular fractions (cytoplasmic fractions (cyto); and mitochondrial fractions (mitochondria)), isolated from a continuous hepatocyte cell line (HepG2) transfected with or without an rAAV vector plasmid carrying a nucleotide sequence encoding the complete native sequence of human PCCA (DTC346). 1× and 2× denote relative volumes of loaded mitochondrial fractions. β-actin (“Actin”) was used as a loading control.

FIG. 6 is a bar graph showing, from left-to-right, fold-change of PCCA AAV9 titer (in genome copies (GC)/mL) as determined by qPCR following triple transfection of a continuous kidney cell line (HEK293) with an rAAV vector plasmid carrying a nucleotide sequence encoding enhanced green fluorescent protein, eGFP (DTC343); an rAAV vector plasmid carrying a nucleotide sequence encoding the complete native sequence of human PCCA (DTC346); or an rAAV vector plasmid carrying a nucleotide sequence encoding a codon-optimized sequence of human PCCA (DTC347, DTC348, or DTC349); each are expressed in a cell co-transfected with an AAV9 capsid-expressing plasmid (pAAV2/9) and a plasmid supplying adenovirus helper functions (pAdHelper). Untransfected cells were used as control and are represented as “no TX”.

FIG. 7 is an image of a DNA alkaline agarose gel of affinity purified AAV9 particles containing PCCA and PCCB-expressing recombinant AAV (rAAV) vector cassette (DTC365), PCCA-expressing recombinant AAV (rAAV) vector cassettes (DTC349, DTC348, DTC347, or DTC346), or eGFP-expressing recombinant AAV (rAAV) vector cassette (DTC343), from left to right, respectively.

FIG. 8 is a bar graph showing fold-change in PCCB RNA expression as determined by RT-qPCR following transfection of a continuous hepatocyte cell line (HepG2) with a control plasmid (plasmid with an empty vector, no TX), an rAAV vector plasmid carrying a nucleotide sequence encoding enhanced green fluorescent protein, eGFP (DTC343), an rAAV vector plasmid carrying a nucleotide sequence encoding the complete native sequence of human PCCB (DTC366), or an rAAV vector plasmid carrying a nucleotide sequence encoding a codon-optimized sequence of human PCCB (DTC367, DTC370, DTC368, DTC369, or DTC371).

FIG. 9A is an image showing protein expression levels, detected by Western blot, in continuous hepatocyte cell line (HepG2) transfected with a control plasmid (plasmid with an empty vector, no TX), an rAAV vector plasmid carrying a nucleotide sequence encoding enhanced green fluorescent protein, eGFP (DTC343), an rAAV vector plasmid carrying a nucleotide sequence encoding the complete native sequence of human PCCB (DTC366), or an rAAV vector plasmid carrying a nucleotide sequence encoding a codon-optimized sequence of human PCCB (DTC367, DTC368, DTC369, DTC370, or DTC371), from lane 1 to lane 8, respectively. β-actin (“Actin”) was used as a loading control.

FIG. 9B is an image showing protein expression levels, detected by Western blot, in continuous hepatocyte cell line (Huh7) transfected with a control plasmid (plasmid with an empty vector, no TX), an rAAV vector plasmid carrying a nucleotide sequence encoding enhanced green fluorescent protein, eGFP (DTC343), an rAAV vector plasmid carrying a nucleotide sequence encoding the complete native sequence of human PCCB (DTC366), or an rAAV vector plasmid carrying a nucleotide sequence encoding a codon-optimized sequence of human PCCB (DTC367, DTC369, DTC368, DTC370, or DTC371), from lane 1 to lane 8, respectively. β-actin (“Actin”) was used as a loading control.

FIG. 10 is an image showing PCCB protein expression levels, detected by Western blot, in whole cell lysates (WCL); subcellular fractions (cytoplasmic fractions (cyto); and mitochondrial fractions (mitochondria)), isolated from a continuous hepatocyte cell line (HepG2) transfected with or without an rAAV vector plasmid carrying a nucleotide sequence encoding the complete native sequence of human PCCB (DTC366). 1× and 2× denote relative volumes of loaded mitochondrial fractions. β-actin (“Actin”) was used as a loading control.

FIG. 11 is a bar graph showing, from left-to-right, fold-change of PCCB AAV9 titer (in genome copies (GC)/mL) as determined by qPCR following triple transfection of a continuous kidney cell line (HEK293) with an rAAV vector plasmid carrying nucleotide sequence encoding enhanced green fluorescent protein, eGFP (DTC343); an rAAV vector plasmid carrying nucleotide sequence encoding the complete native sequence of human PCCB (DTC366); or an rAAV vector plasmid carrying a nucleotide sequence encoding a codon-optimized sequence of human PCCA (DTC367, DTC368, DTC369, DTC370, or DTC371); each are expressed in a cell co-transfected with an AAV9 capsid expressing plasmid (pAAV2/9) and a plasmid supplying adenovirus helper functions (pAdHelper). Untransfected cells were used as control and are represented as “no TX”.

FIG. 12 is an image of a DNA alkaline agarose gel of affinity purified AAV9 particles containing control plasmid (plasmid with an empty vector, no TX), an eGFP-expressing recombinant AAV (rAAV) vector cassette (DTC343), or a PCCB-expressing recombinant AAV (rAAV) vector cassette (DTC366, DTC367, DTC368, DTC369, DTC370, or DTC371), from left to right, respectively.

FIG. 13 is a bar graph showing the percentage of human PCCA protein expression relative to mouse endogenous PCCA expression in wild-type FVB mice following treatment with rAAV encoding the complete native sequence of human PCCA (DTC346), or a codon-optimized sequence of human PCCA (DTC347, DTC348, or DTC349). Error bars represent standard deviations.

FIG. 14 is a bar graph showing the concentration (in nmol/g protein) of human wild-type PCCA protein expressed in a hypomorphic PCCA mouse model (A138T mutant) following treatment with rAAV encoding the complete native sequence of human PCCA (DTC346) or a codon-optimized sequence of human PCCA (DTC347, DTC348, or DTC349). The listed percentage number denotes human wildtype PCCA expression calculated as a percentage to endogenous PCCA protein level of wildtype FVB mice. Error bars represent standard deviations.

FIG. 15 is a bar graph of human PCCA activity in a hypomorphic PCCA mouse model (A138T mutant) following treatment with rAAV encoding the complete native sequence of human PCCA (DTC346), as measured by mean counts per minute (CPM) data. Error bars represent standard deviations. The listed percentage numbers describe the CPM data relative to those observed in wild-type FVB mice. ** denotes p<0.01 in comparison to PBS-treated group using Dunnett multiple comparison test.

FIGS. 16A-C are bar graphs showing concentrations of known biomarkers of propionic acidemia in a hypomorphic PCCA mouse model (A138T mutant) following treatment with rAAV encoding the complete native sequence of human PCCA (DTC346). FIG. 16A is a bar graph showing plasma concentrations of C3 (propionylcarnitine) in a hypomorphic PCCA mouse model (A138T mutant) following treatment with rAAV encoding the complete native sequence of human PCCA (DTC346). FIG. 16B is bar graph of plasma C3/C2 concentration ratio (propionylcarnitine/acetylcarnitine) in a hypomorphic PCCA mouse model (A138T mutant) following treatment with rAAV encoding the complete native sequence of human PCCA (DTC346). FIG. 16C is a bar graph showing plasma concentrations of 2-methylcitrate (2MC) in a hypomorphic PCCA mouse model (A138T mutant) following treatment with rAAV encoding the complete native sequence of human PCCA (DTC346). Pre indicates 0 to 14 days before injection of rAAV, 2W indicates 2 weeks after rAAV injection, 3W indicates 3 weeks after rAAV injection, 4W indicates 4 weeks after rAAV injection, and 6W indicates 6 weeks after rAAV injection. Error bars represent standard deviations. ** denotes p<0.01 in comparison to PBS-treated group and *** denotes p<0.001 in comparison to PBS-treated group using Dunnett multiple comparison test.

### DETAILED DESCRIPTION OF THE INVENTION

This invention provides a range of novel agents and compositions to be used for therapeutic applications. The nucleic acid sequences, vectors, recombinant viruses, and associated compositions of this invention can be used for ameliorating, preventing, or treating PA as described herein.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Adeno-associated virus (AAV): A small, replication-defective, non-enveloped virus that infects humans and some other primate species. AAV is not known to cause disease and elicits a very mild immune response. Gene therapy vectors that utilize AAV can infect both dividing and quiescent cells and can persist in an extrachromosomal state without integrating into the genome of the host cell. These features make AAV an attractive viral vector for gene therapy. There are currently 12 recognized serotypes of AAV (AAV1-12).

Administration/Administer: To provide or give a subject an agent, such as a therapeutic agent (e.g., a recombinant AAV), by any effective route. Exemplary routes of administration include, but are not limited to, injection (such as subcutaneous, intramuscular, intradermal, intraperitoneal, and intravenous), oral, intraductal, sublingual, rectal, transdermal, intranasal, vaginal and inhalation routes.

Codon-optimized: A “codon-optimized” nucleic acid refers to a nucleic acid sequence that has been altered such that the codons are optimal for expression in a particular system (such as a particular species or group of species). For example, a nucleic acid sequence can be optimized for expression in mammalian cells or in a particular mammalian species (such as human cells). Codon optimization does not alter the amino acid sequence of the encoded protein.

Enhancer: A nucleic acid sequence that increases the rate of transcription by increasing the activity of a promoter.

Intron: A stretch of DNA within a gene that does not contain coding information for a protein. Introns are removed before translation of a messenger RNA.

Inverted terminal repeat (ITR): Symmetrical nucleic acid sequences in the genome of adeno-associated viruses required for efficient replication. ITR sequences are located at each end of the AAV DNA genome. The ITRs serve as the origins of replication for viral DNA synthesis and are essential cis components for generating AAV integrating vectors.

Isolated: An “isolated” biological component (such as a nucleic acid molecule, protein, virus or cell) has been substantially separated or purified away from other biological components in the cell or tissue of the organism, or the organism itself, in which the component naturally occurs, such as other chromosomal and extra-chromosomal DNA and RNA, proteins and cells. Nucleic acid molecules and proteins that have been “isolated” include those purified by standard purification methods. The term also embraces nucleic acid molecules and proteins prepared by recombinant expression in a host cell as well as chemically synthesized nucleic acid molecules and proteins.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carrier: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds, molecules or agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating or ameliorating a disease: “Preventing” a disease (such as PA) refers to inhibiting the full development of a disease. “Treating” refers to a therapeutic intervention that ameliorates a sign or symptom of a disease or pathological condition (such as PA) after it has begun to develop. “Ameliorating” refers to the reduction in the number or severity of signs or symptoms of a disease (such as PA).

Promoter: A region of DNA that directs/initiates transcription of a nucleic acid (e.g., a gene). A promoter includes necessary nucleic acid sequences near the start site of transcription. Many promoter sequences are known to the person skilled in the art and even a combination of different promoter sequences in artificial nucleic acid molecules is possible. As used herein, gene-specific endogenous promoter refers to native promoter element that regulates expression of the endogenous gene of interest. In an exemplary embodiment, a PCCA gene-specific endogenous promoter regulates expression of a PCCA gene. In another exemplary embodiment, a PCCB gene-specific endogenous promoter regulates expression of a PCCB gene.

Purified: The term “purified” does not require absolute purity; rather, it is intended as a relative term. Thus, for example, a purified peptide, protein, virus, or other active compound is one that is isolated in whole or in part from naturally associated proteins and other contaminants. In certain embodiments, the term “substantially purified” refers to a peptide, protein, virus or other active compound that has been isolated from a cell, cell culture medium, or other crude preparation and subjected to fractionation to remove various components of the initial preparation, such as proteins, cellular debris, and other components.

Recombinant: A recombinant nucleic acid molecule is one that has a sequence that is not naturally occurring or has a sequence that is made by an artificial combination of two otherwise separated segments of sequence. This artificial combination can be accomplished by chemical synthesis or by the artificial manipulation of isolated segments of nucleic acid molecules, such as by genetic engineering techniques.

Similarly, a recombinant virus is a virus comprising sequence (such as genomic sequence) that is non-naturally occurring or made by artificial combination of at least two sequences of different origin. The term “recombinant” also includes nucleic acids, proteins and viruses that have been altered solely by addition, substitution, or deletion of a portion of a natural nucleic acid molecule, protein or virus. As used herein, “recombinant AAV” refers to an AAV particle in which a recombinant nucleic acid molecule such as a recombinant nucleic acid molecule encoding PCCA and/or a recombinant nucleic acid molecule encoding PCCB has been packaged.

Sequence identity: The identity or similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and mouse sequences), compared to species more distantly related (such as human and C. elegans sequences).

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970: Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Rio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J Mol. Biol. 215:403-10, 1990) is available from several sources, including the National Center for Biological Information (NCBI) and on the internet, for use in connection with the sequence analysis programs blastp, blastn, blastx, tblastn and tblastx. Additional information can be found at the NCBI web site.

Serotype: A group of closely related microorganisms (such as viruses) distinguished by a characteristic set of antigens.

Stuffer sequence: Refers to a sequence of nucleotides contained within a larger nucleic acid molecule (such as a vector) that is typically used to create desired spacing between two nucleic acid features (such as between a promoter and a coding sequence), or to extend a nucleic acid molecule so that it is of a desired length. Stuffer sequences do not contain protein coding information and can be of unknown/synthetic origin and/or unrelated to other nucleic acid sequences within a larger nucleic acid molecule.

Subject: Living multi-cellular vertebrate organisms, a category that includes human and non-human mammals.

Synthetic: Produced by artificial means in a laboratory, for example a synthetic nucleic acid can be chemically synthesized in a laboratory.

Untranslated region (UTR): A typical mRNA contains a 5′ untranslated region (“5′ UTR”) and a 3′ untranslated region (3′ UTR) upstream and downstream, respectively, of the coding region (see Mignone F. et. al., (2002) Genome Biol 3:REVIEWS0004).

Therapeutically effective amount: A quantity of a specified pharmaceutical or therapeutic agent (e.g., a recombinant AAV) sufficient to achieve a desired effect in a subject, or in a cell, being treated with the agent. The effective amount of the agent will be dependent on several factors, including, but not limited to the subject or cells being treated, and the manner of administration of the therapeutic composition.

Vector: A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and translation of inserted gene or genes. In some embodiments herein, the vector is an AAV vector.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Viral Vectors:

In some aspects, the present disclosure provides adeno-associated virus (AAV) vector that contains a packaged genome that comprises an AAV 5′-ITR, a promoter sequence, a partial or complete coding sequence for PCCA or an isoform thereof, or a functional fragment or functional variant thereof, and an AAV 3′-ITR.

In certain embodiments, the present disclosure provides an AAV vector that contains a packaged genome that comprises an AAV 5′-ITR, a promoter sequence, a truncated or complete nucleotide sequence of human PCCA 5′-UTR, a partial or complete coding sequence for PCCA or an isoform thereof, or a functional fragment or functional variant thereof, a truncated or complete human PCCA 3′-UTR nucleotide sequence, and an AAV 3′-ITR.

In some aspects, the present disclosure provides AAV vector that contains a packaged genome that comprises an AAV 5′-ITR, a promoter sequence, a partial or complete coding sequence for PCCB or an isoform thereof, or a functional fragment or functional variant thereof, and an AAV 3′-ITR.

In certain embodiments, the present disclosure provides an AAV vector that contains a packaged genome that comprises an AAV 5′-ITR, a promoter sequence, a truncated or complete nucleotide sequence of human PCCB 5′-UTR, a partial or complete coding sequence for PCCB or an isoform thereof, or a functional fragment or functional variant thereof, a truncated or complete human PCCB 3′-UTR nucleotide sequence, and an AAV 3′-ITR.

In some embodiments, the packaged genome may further comprise an enhancer, an intron, a consensus Kozak sequence, and/or a polyadenylation signal as described herein. In some embodiments, the recombinant vector can further include one or more stuffer nucleic acid sequences. In one embodiment, a stuffer nucleic acid sequence is situated between the intron and the partial or complete coding sequence for PCCA or PCCB.

In various embodiments described herein, the recombinant virus vector is an AAV vector. The AAV vector can be an AAV vector of serotype 1, 2, 3, 4, 5, 6, 7, 8, 9, 10, 11 or 12 (i.e., AAV1, AAV2, AAV3, AAV4, AAV5, AAV6, AAV7, AAV8, AAV9, AAV10, AAV11 or AAV12), as well as any one of the more than 100 variants isolated from human and nonhuman primate tissues. See, e.g., Choi et al., 2005, Curr Gene Ther. 5: 299-310, 2005 and Gao et al., 2005, Curr Gene Ther. 5: 285-297. AAV vectors of any serotype may be used in the present invention, and the selection of AAV serotype will depend in part on the cell type(s) that are targeted for gene therapy. For treatment of PA, the liver is one of the relevant target organs. In some embodiments, the AAV vector is selected from serotype 9 (AAV9), serotype 8 (AAV8), or variant thereof. In an exemplary embodiment, the AAV vector is serotype 9 (AAV9) or a variant thereof.

In some embodiments, the recombinant AAV vector includes an AAV ITR sequence, which functions as both the origin of vector DNA replication and the packaging signal of the vector genome, when AAV and adenovirus helper functions are provided in trans. Additionally, the ITRs serve as the target for single-stranded endonucleatic nicking by the large Rep proteins, resolving individual genomes from replication intermediates.

In some embodiments, the 5′-ITR sequence is from AAV2. In some embodiments, the 3′-ITR sequence is from AAV2. In some embodiments, the 5′-ITR sequence and the 3′-ITR sequence are from AAV2. In some embodiments, the 5′-ITR sequence and/or the 3′-ITR sequence are from AAV2 and comprise or consist of SEQ ID NO: 15. In other embodiments, the 5′-ITR sequence and/or the 3′-ITR sequence are from a non-AAV2 source.

In some exemplary embodiments, the AAV vector is an AAV serotype 9 (AAV9) vector, and the vector includes an enhancer, a promoter, an intron, a coding sequence for PCCA or PCCB, and a polyadenylation signal described herein. In some embodiments, the AAV9 vector further includes two AAV2, AAV8, or AAV9 inverted terminal repeat (ITR) sequences: one 5′ of the enhancer and one 3′ of the polyadenylation signal. In an exemplary embodiment, the AAV9 vector includes two AAV2 inverted terminal repeat (ITR) sequences: one 5′ of the enhancer and one 3′ of the polyadenylation signal. In some embodiments, the AAV2 ITR sequences comprise or consist of SEQ ID NO: 15. In another exemplary embodiment, the AAV9 vector includes two AAV9 inverted terminal repeat (ITR) sequences: one 5′ of the enhancer and one 3′ of the polyadenylation signal.

### Promoter:

In various aspects described herein, viral vectors are provided, which contain a packaged genome which comprises a promoter sequence which helps drive and regulate transgene expression, e.g., expression of PCCA or PCCB. In certain embodiments, the promoter is selected from a chicken β-actin (CBA) promoter, a cytomegalovirus (CMV) immediate early gene promoter, a transthyretin (TTR) promoter, a thyroxine binding globulin (TBG) promoter, an alpha-1 anti-trypsin (A1AT) promoter, a CAG promoter, a PCCA gene-specific endogenous promoter, and a PCCB gene-specific endogenous promoter. In exemplary embodiments, the promoter sequence is located between the selected 5′-ITR sequence and the partial or complete coding sequence for PCCA or PCCB. In some embodiments, the promoter sequence is located downstream of an enhancer sequence. In some embodiments, the promoter sequence is located upstream of an intron sequence. In some embodiments, the promoter sequence is located between the selected 5′-ITR sequence and the truncated or complete nucleotide sequence of human PCCA or human PCCB 5′-untranslated region (UTR).

In some illustrative embodiments, a ubiquitous chicken β-actin promoter (CBA), which may optionally be located downstream of a CMV immediate early enhancer (CMV IE), is used. In some certain embodiments, a packaged genome described herein comprises the native PCCA or PCCB promoter element. In some illustrative embodiments, a packaged genome described herein comprises a PCCA gene-specific endogenous promoter comprising a nucleotide sequence of at least 15 continuous nucleotides, which is at least 95% identical to an equal length region of SEQ ID NO: 34 when the partial or complete coding sequence in the packaged genome is for PCCA. In certain embodiments, a packaged genome described herein comprises a PCCA gene-specific endogenous promoter comprising a nucleotide sequence of about 15 continuous nucleotides (for example about 30, about 45, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 325, about 350, about 375, about 400, about 425, about 450, about 475, about 500, about 525, about 550, about 575, about 600, about 625, about 650, about 675, about 700, about 725, or about 750), which is at least 95% identical to an equal length region of SEQ ID NO: 34. In some illustrative embodiments, a a packaged genome described herein comprises a PCCA gene-specific endogenous promoter comprising a nucleotide sequence of at least 15 continuous nucleotides, which is 100% identical to an equal length region of SEQ ID NO: 34 when the partial or complete coding sequence in the vector genome is for PCCA.

In some illustrative embodiments a packaged genome described herein comprises a PCCB gene-specific endogenous promoter comprising a nucleotide sequence of at least 15 continuous nucleotides, which is at least 95% identical to an equal length region of SEQ ID NO: 36 when the partial or complete coding sequence in the vector genome is for PCCB. In certain embodiments, a packaged genome described herein comprises a PCCB gene-specific endogenous promoter comprising a nucleotide sequence of about 15 continuous nucleotides (for example about 30, about 45, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 325, about 350, about 375, about 400, about 425, or about 450), which is at least 95% identical to an equal length region of SEQ ID NO: 36. In some illustrative embodiments a packaged genome described herein comprises a PCCB gene-specific endogenous promoter comprising a nucleotide sequence of at least 15 continuous nucleotides, which is 100% identical to an equal length region of SEQ ID NO: 36 when the partial or complete coding sequence in the vector genome is for PCCB.

In some embodiments, the promoter is selected from a chicken β-actin (CBA) promoter, a cytomegalovirus (CMV) immediate early gene promoter, a transthyretin (TTR) promoter, a thyroxine binding globulin (TBG) promoter, an alpha-1 anti-trypsin (A1AT) promoter, and a CAG promoter. In an exemplary embodiment, the promoter is the CBA promoter. In one embodiment, the CBA promoter comprises or consists of SEQ ID NO: 18.

In addition to a promoter, a packaged genome may contain other appropriate transcription initiation, termination, enhancer sequence, and efficient RNA processing signals. As described in further detail below, such sequences include splicing and polyadenylation (poly A) signals, regulatory elements that enhance expression (i.e., WPRE), sequences that stabilize cytoplasmic mRNA, sequences that enhance translation efficiency (i.e., the Kozak consensus sequence), and sequences that enhance protein stability.

In some embodiments, the packaged genome further comprises a consensus Kozak sequence. In some embodiments, the consensus Kozak sequence is located downstream of an intron sequence. In one embodiment, the consensus Kozak sequence is GCCGCC (SEQ ID NO: 24). As will be understood by those skilled in the art, the consensus Kozak sequence is typically located immediately upstream of a coding sequence; in this case, immediately upstream of a partial or complete coding sequence for PCCA or PCCB. As will be appreciated by the skilled artisan, the consensus Kozak sequence can be considered to share an ATG residue corresponding to the start codon of the therapeutic polypeptide, e.g., PCCA or PCCB. For the simplicity of disclosure, the consensus Kozak sequence, as described herein, comprises a six-nucleotide sequence corresponding to the region not shared with the therapeutic polypeptide, e.g., PCCA or PCCB.

### Untranslated Region (UTR):

5′-untranslated region (UTR) from endogenous gene-specific mRNA have been known to play an important role in optimizing transgene production by competing with cellular transcripts for translation initiation factors and ribosomes, increasing mRNA half-life by minimizing mRNA decay or post-transcriptional gene silencing, and avoiding deleterious interactions with regulatory proteins or inhibitory RNA secondary structures (see Chiba, Y., and Green, P. (2009). J. Plant Biol. 52, 114-124, Moore, M. J., and Proudfoot, N. J. (2009). Cell 136, 688-700, Jackson, R. J., et. al. (2010). Nat. Rev. Mol. Cell Biol. 11, 113-127). The 3′-untranslated region (3′-UTR), situated downstream of the protein coding sequence, has been found to be involved in numerous regulatory processes including transcript cleavage, stability and polyadenylation, translation and mRNA localisation. They are thus critical in determining the fate of an mRNA (see Barrett, L. W., et. al. (2012). Cell Mol Life Sci. November; 69(21): 3613-3634). In certain embodiments, the present disclosure provides an rAAV that contains a packaged genome that comprises a truncated or complete nucleotide sequence of human PCCA or PCCB 5′-UTR. In certain embodiments, the present disclosure provides an rAAV that contains a packaged genome that comprises a truncated or complete nucleotide sequence of human PCCA or PCCB 3′-UTR.

In certain embodiments, the present disclosure provides an rAAV that contains a packaged genome that comprises an AAV 5′-ITR, a promoter sequence, a truncated or complete nucleotide sequence of human PCCA 5′-UTR, a partial or complete coding sequence for PCCA or an isoform thereof, or a functional fragment or functional variant thereof, a truncated or complete human PCCA 3′-UTR nucleotide sequence, and an AAV 3′-inverted terminal repeat sequence (ITR).

In certain embodiments, the present disclosure provides an rAAV that contains a packaged genome that comprises an AAV 5′-ITR, a promoter sequence, a truncated or complete nucleotide sequence of human PCCB 5′-UTR, a partial or complete coding sequence for PCCB or an isoform thereof, or a functional fragment or functional variant thereof, a truncated or complete human PCCB 3′-UTR nucleotide sequence, and an AAV 3′-ITR.

In certain embodiments, the packaged genome comprises the complete human PCCA 5′-UTR represented by SEQ ID NO: 30. In certain embodiments, the packaged genome comprises complete human PCCB 5′-UTR represented by SEQ ID NO: 32.

In certain embodiments, the packaged genome comprises a truncated native human PCCA 5′-UTR. In certain embodiments, the truncated human PCCA 5′-UTR comprises a nucleotide sequence of about 50 continuous nucleotides to about 1400 continuous nucleotides (e.g., about 75, about 100, about 125, about 150, about 175, about 200, about 250, about 275, about 300, about 325, about 350, about 400, about 425, about 450, about 475, about 500, about 525, about 550, about 575, about 600, about 625, about 650, about 675, about 700, about 725, about 750, about 775, about 800, about 825, about 850, about 900, about 925, about 950, about 975, about 1000, about 1025, about 1050, about 1075, about 1100, about 1125, about 1150, about 1175, about 1200, about 1225, about 1250, about 1275, about 1300, about 1325, or about 1350), which is at least 95% identical to an equal length region of SEQ ID NO: 30.

In certain embodiments, the packaged genome comprises a truncated native human PCCB 5′-UTR. In certain embodiments, the truncated human PCCB 5′-UTR comprises a nucleotide sequence of about 50 continuous nucleotides to about 1400 continuous nucleotides (e.g., about 75, about 100, about 125, about 150, about 175, about 200, about 250, about 275, about 300, about 325, about 350, about 400, about 425, about 450, about 475, about 500, about 525, about 550, about 575, about 600, about 625, about 650, about 675, about 700, about 725, about 750, about 775, about 800, about 825, about 850, about 900, about 925, about 950, about 975, about 1000, about 1025, about 1050, about 1075, about 1100, about 1125, about 1150, about 1175, about 1200, about 1225, about 1250, about 1275, about 1300, about 1325, or about 1350), which is at least 95% identical to an equal length region of SEQ ID NO: 32.

In certain embodiments, the packaged genome comprises complete human PCCA 3′-UTR represented by SEQ ID NO: 31. In certain embodiments, the packaged genome comprises complete human PCCB 3′-UTR represented by SEQ ID NO: 33.

In certain embodiments, the packaged genome comprises a truncated native human PCCA 3′-UTR. In certain embodiments, the truncated human PCCA 3′-UTR comprises a nucleotide sequence of about 15 continuous nucleotides (e.g., about 30, about 45, about 60, about 70, about 80, about 90, about 100, about 110, about 120, about 130, about 140, about 150, about 160, about 170, about 180, about 190, about 200, about 210, about 220, about 230, about 240, about 250, about 260, about 270), which is at least 95% identical to an equal length region of SEQ ID NO: 31.

In certain embodiments, the packaged genome comprises a truncated native human PCCB 3′-UTR. In certain embodiments, the truncated human PCCB 3′-UTR comprises a nucleotide sequence of about 15 continuous nucleotides (e.g., about 30, about 45, about 60, about 70, about 80, about 90, about 100, about 110, about 120, about 130, about 140, or about 150), which is at least 95% identical to an equal length region of SEQ ID NO: 33.

### PCCA or PCCB Polypeptides:

As described herein, aspects of the invention provide recombinant vectors that include a packaged genome that comprises an AAV 5′-ITR, a promoter sequence, a partial or complete coding sequence for PCCA or an isoform thereof, or a functional fragment or functional variant thereof, and an AAV 3′-ITR. Further described herein are recombinant vectors that include a packaged genome that comprises an AAV 5′-ITR, a promoter sequence, a partial or complete coding sequence for PCCB or an isoform thereof, or a functional fragment or functional variant thereof, and an AAV 3′-ITR.

In one embodiment, the partial or complete coding sequence for PCCA or PCCB is a wild-type coding sequence. As used herein, the term “wild-type” refers to a biopolymer (e.g., a polypeptide sequence or polynucleotide sequence) that is the same as the biopolymer (e.g., polypeptide sequence or polynucleotide sequence) that exists in nature.

In an alternative embodiment, the partial or complete coding sequence for PCCA or PCCB is a codon-optimized coding sequence. In one embodiment, the partial or complete coding sequence for PCCA or PCCB is codon-optimized for expression in humans.

In various embodiments described herein, vectors are provided that contain a packaged genome that comprise a coding sequence for PCCA or PCCB. The polypeptides delivered with the vectors described herein encompass PCCA and PCCB polypeptides that may be useful in the treatment of mammals, including humans.

In some embodiments, the polypeptide expressed with a vector described herein is PCCA (SEQ ID NO: 16; GenBank Accession No. NP_000273.2; 728 amino acids) or a functional fragment, functional variant, or functional isoform thereof. In some embodiments, the polypeptide expressed with a vector described herein is PCCA and comprises or consists of SEQ ID NO: 16.

In one embodiment, the PCCA polypeptide is encoded by the wild-type coding sequence shown in SEQ ID NO: 1. In another embodiment, a coding sequence expressing a natural isoform or variant of PCCA may be used, such as those shown in UniProtKB/Swiss-Prot Accession Nos. P05165-1 (SEQ ID NO: 25), P05165-2 (SEQ ID NO: 26), and P05165-3 (SEQ ID NO: 27). In alternative embodiments, the PCCA polypeptide is encoded by a codon-optimized coding sequence. In some embodiments, the PCCA polypeptide is encoded by a codon-optimized coding sequence that is less than 80% identical to the wild-type coding sequence shown in SEQ ID NO: 1. In some exemplary embodiments, the PCCA polypeptide is encoded by a codon-optimized coding sequence selected from SEQ ID NOs: 2-6. In some embodiments, the coding sequence for PCCA may further comprise a stop codon (TGA, TAA, or TAG) at the 3′ end.

In some embodiments, the polypeptide expressed with a vector described herein is PCCB (SEQ ID NO: 17; GenBank Accession No. NP_000523.2; 539 amino acids) or a functional fragment, functional variant, or functional isoform thereof. In some embodiments, the polypeptide expressed with a vector described herein is PCCB and comprises or consists of SEQ ID NO: 17.

In one embodiment, the PCCB polypeptide is encoded by the wild-type coding sequence shown in SEQ ID NO: 7. In another embodiment, a coding sequence expressing a natural isoform or variant of PCCB may be used, such as those shown in UniProtKB/Swiss-Prot Accession Nos. P05166-1 (SEQ ID NO: 28) and P05166-2 (SEQ ID NO: 29). In alternative embodiments, the PCCB polypeptide is encoded by a codon-optimized coding sequence. In some embodiments, the PCCB polypeptide is encoded by a codon-optimized coding sequence that is less than 80% identical to the wild-type coding sequence shown in SEQ ID NO: 7. In some exemplary embodiments, the PCCB polypeptide is encoded by a codon-optimized coding sequence selected from SEQ ID NOs: 8-12. In some embodiments, the coding sequence for PCCB may further comprise a stop codon (TGA, TAA, or TAG) at the 3′ end.

In various aspects, the invention may be used to deliver fragments, variants, isoforms, or fusions of the PCCA or PCCB polypeptides described herein.

In some embodiments, the invention may be used to deliver fragments of the PCCA or PCCB polypeptides, which comprise at least 50, 100, 150, 200, 250, 300, 350, 400, 450, 500, 550, or 600 amino acid residues and retain one or more activities associated with the full-length polypeptide (e.g., catalytic activity in the case of an enzyme). Such fragments may be obtained by recombinant techniques that are routine and well-known in the art. Moreover, such fragments may be tested for catalytic activity by routine in vitro assays known to the skilled artisan. For instance, propionyl-CoA carboxylase (PCC) activity can be assayed by (1) diluting the polypeptide in 10 mM phosphate buffer (pH 7.0) containing 1 mM 2-mercaptoethanol and 0.1 mg/ml of bovine serum albumin, (2) taking the standard reaction mixture containing 50 mM Tris-HCl pH 8.0, 5 mM glutathione, 2 mM ATP, 100 mM KCl, 10 mM MgCl₂, 10 mM [¹⁴C]-bicarbonate (specific activity 12.4 mCi/mmol), 3 mM propionyl-CoA, and incubating enzyme at 37 degrees C. for 15 min, (3) stopping the reaction by addition of 10% trichloroacetic acid, (4) centrifuging at 200×g, (5) drying an aliquot under a heat lamp, (6) dissolving in water, and (7) counting in Aquasol, wherein one unit of enzyme activity is defined as that amount of enzyme catalyzing the fixation of 1 pmol of bicarbonate/min at 37 degrees C. See Kalousek et al., 1980, J Biol Chem 255(1): 60-65 and Hsia et al., 1973 J. Pediatr. 83: 625-628 for a description of PCC activity assays. The invention further includes nucleic acid molecules which encode the above-described polypeptide fragments.

In some embodiments, the invention may be used to deliver variants of the PCCA or PCCB polypeptides. In some embodiments, the variant polypeptides may be at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or 100%) identical to the wild-type therapeutic polypeptide, e.g., a wild-type PCCA polypeptide of SEQ ID NO: 16 or a wild-type PCCB polypeptide of SEQ ID NO: 17. In some embodiments, the variant therapeutic polypeptides may have at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 different residues as compared to the respective wild-type polypeptide. Such variants may be obtained by recombinant techniques that are routine and well-known in the art. Moreover, such variants may be tested for catalytic activity by routine in vitro assays known to the skilled artisan. See, e.g., Kalousek et al., 1980, J Biol Chem 255(1): 60-65 and Hsia et al., 1973 J. Pediatr. 83: 625-628 for a description of propionyl-CoA carboxylase activity assays. The invention further includes nucleic acid molecules which encode the above described therapeutic polypeptide variants.

### Novel Codon-Optimized Sequences:

In some aspects, the present disclosure provides novel codon-optimized nucleic acid sequences encoding PCCA. In one embodiment, the codon-optimized nucleic acid sequence encoding PCCA is less than 80% identical to the wild-type coding sequence shown in SEQ ID NO: 1. In some embodiments, the codon-optimized nucleic acid sequence encoding PCCA is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or 100%) identical to SEQ ID NOs: 2-6. In some embodiments, the codon-optimized nucleic acid sequence encoding PCCA is 100% identical to a sequence selected from SEQ ID NOs: 2-6. In some embodiments, the present disclosure provides nucleic acid sequences which are less than 80% identical to the wild-type coding sequence shown in SEQ ID NO: 1 and are at least 80%, 85%, 90%, 91%, 92%, 93%, 94%, 95%, 96%, 97%, 98%, 99% or more identical to SEQ ID NOs: 2-6. In exemplary embodiments, the present disclosure provides a nucleic acid sequence encoding PCCA selected from SEQ ID NOs: 2-6. Further provided are fragments of the nucleic acid sequences shown in SEQ ID NOs: 2-6 which encode a polypeptide having functional PCCA activity. In some embodiments, the nucleic acid sequence encoding PCCA may further comprise a stop codon (TGA, TAA, or TAG) at the 3′ end.

In some aspects, the present disclosure provides novel codon-optimized nucleic acid sequences encoding PCCB. In one embodiment, the codon-optimized nucleic acid sequence encoding PCCB is less than 80% identical to the wild-type coding sequence shown in SEQ ID NO: 7. In some embodiments, the codon-optimized nucleic acid sequence encoding PCCB is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or 100%) identical to SEQ ID NOs: 8-12. In some embodiments, the codon-optimized nucleic acid sequence encoding PCCB is 100% identical to a sequence selected from SEQ ID NOs: 8-12. In some embodiments, the present disclosure provides nucleic acid sequences which are less than 80% identical to the wild-type coding sequence shown in SEQ ID NO: 7 and are at least 80%, 85%, 90%, 91%, 92%, 93%, 94%, 95%, 96%, 97%, 98%, 99% or more identical to SEQ ID NOs: 8-12. In exemplary embodiments, the present disclosure provides a nucleic acid sequence encoding PCCB selected from SEQ ID NOs: 8-12. Further provided are fragments of the nucleic acid sequences shown in SEQ ID NOs: 8-12 which encode a polypeptide having functional PCCB activity. In some embodiments, the nucleic acid sequence encoding PCCB may further comprise a stop codon (TGA, TAA, or TAG) at the 3′ end.

### Vector Genome Elements:

In some embodiments, the rAAV contains a packaged vector genome that further comprises one or more enhancer sequences. In one embodiment, the enhancer is selected from a cytomegalovirus immediate early gene (CMV) enhancer, a transthyretin enhancer (enTTR), a chicken β-actin (CBA) enhancer, an En34 enhancer, and an ApoE enhancer. In an exemplary embodiment, the enhancer is the CMV enhancer. In one embodiment, the CMV enhancer comprises or consists of SEQ ID NO: 19.

In some embodiments, the rAAV contains a packaged vector genome that further comprises one or more intron sequences. In one embodiment, the intron is selected from an SV40 Small T intron, a rabbit hemoglobin subunit beta (rHBB) intron, a human beta globin IVS2 intron, a Promega chimeric intron, or an hFIX intron. In one exemplary embodiment, the intron is the SV40 Small T intron. In one embodiment, the SV40 Small T intron sequence comprises or consists of SEQ ID NO: 20. In another exemplary embodiment, the intron is the rHBB intron. In one embodiment, the rHBB intron sequence comprises or consists of SEQ ID NO: 21.

In some embodiments, the rAAV contains a packaged vector genome that further comprises a polyadenylation signal sequence. In one embodiment, the polyadenylation signal sequence is selected from a bovine growth hormone (BGH) polyadenylation signal sequence, an SV40 polyadenylation signal sequence, a rabbit beta globin polyadenylation signal sequence, a PCCA gene-specific endogenous polyadenylation signal sequence, a PCCB gene-specific endogenous polyadenylation signal sequence. In an exemplary embodiment, the polyadenylation signal sequence is the bovine growth hormone (BGH) polyadenylation signal sequence. In one embodiment, the BGH polyadenylation signal sequence comprises or consists of SEQ ID NO: 22. In another exemplary embodiment, the polyadenylation signal sequence is the SV40 polyadenylation signal sequence. In one embodiment, the SV40 polyadenylation signal sequence comprises or consists of SEQ ID NO: 23. In one embodiment, the polyadenylation signal sequence is a gene-specific endogeneous polyadenylation sequence. In an exemplary embodiment, the polyadenylation signal sequence is a PCCA gene-specific endogenous polyadenylation signal sequence when the partial or complete coding sequence in the vector genome is for PCCA. In certain embodiments, the PCCA gene-specific endogenous polyadenylation signal sequence comprises a nucleotide sequence of at least 15 continuous nucleotides, which is at least 95% identical to an equal length region of SEQ ID NO: 35. In certain embodiments, the PCCA gene-specific endogenous polyadenylation signal sequence comprises a nucleotide sequence of about 15 continuous nucleotides (e.g., about 20, about 30, about 40, about 50, about 60, about 70, about 80, about 90, about 100, about 110, about 120, about 130, about 140, about 150, or about 160), which is at least 95% identical to an equal length region of SEQ ID NO: 35. In another exemplary embodiment, the polyadenylation signal sequence is a PCCB gene-specific endogenous polyadenylation signal sequence when the partial or complete coding sequence in the vector genome is for PCCB. In certain embodiments, the PCCB gene-specific endogenous polyadenylation signal sequence comprises a nucleotide sequence of at least 15 continuous nucleotides, which is at least 95% identical to an equal length region of SEQ ID NO: 37. In certain embodiments, the PCCA gene-specific endogenous polyadenylation signal sequence comprises a nucleotide sequence of about 15 continuous nucleotides (e.g., about 20, about 30, about 40, about 50, about 60, about 70, about 80, about 90, or about 100), which is at least 95% identical to an equal length region of SEQ ID NO: 37.

### AAV Capsids:

In another aspect, the present disclosure provides rAAV useful as agents for gene therapy in the treatment of PA, wherein said rAAV comprises an AAV capsid, and a vector genome as described herein packaged therein. In some embodiments, the AAV capsid is from an AAV of serotype 1, 2, 3, 4, 5, 6, 7, 8, 9, 10, 11, 12, rh10, or hu37 (i.e., AAV1, AAV2, AAV3, AAV4, AAV5, AAV6, AAV7, AAV9, AAV10, AAV11, AAV12, AAVrh10, or AAVhu37). In an exemplary embodiment, the AAV vector is an AAV serotype 9 (AAV9) vector, an AAV9 variant vector, an AAV serotype 8 (AAV8) vector, or an AAV serotype 2 (AAV2) vector.

The AAV9 capsid is a self-assembled AAV capsid composed of multiple AAV9 vp proteins. The AAV9 vp proteins are typically expressed as alternative splice variants encoded by a nucleic acid sequence of SEQ ID NO: 13 or a sequence that is at least 70%, at least 75%, at least 80%, at least 85%, at least 90%, at least 95%, at least 97%, at least 99% thereto, which encodes the vp1 amino acid sequence of SEQ ID NO: 14 (GenBank Accession: AAS99264). These splice variants result in proteins of different length of SEQ ID NO: 14. In certain embodiments, an AAV9 capsid includes an AAV having an amino acid sequence which is 99% identical to AAS99264 or 99% identical to SEQ ID NO: 14. See also U.S. Pat. No. 7,906,111, and WO/2005/033321. As used herein, an AAV9 variant includes those described in, e.g., WO/2016/049230, U.S. Pat. No. 8,927,514, US Patent Publication No. 2015/0344911, and U.S. Pat. No. 8,734,809.

As indicated herein, the AAV9 sequences and proteins are useful in the production of rAAV. However, in other embodiments, another AAV capsid is selected. Tissue specificity is determined by the capsid type. AAV serotypes which transduce a suitable target (e.g., liver, muscle, lung, or CNS) may be selected as sources for capsids of AAV viral vectors including, e.g., AAV1, AAV2, AAV3, AAV4, AAV5, AAV6, AAV6.2, AAV7, AAV8, AAV9, AAVrh10, AAVrh64R1, AAVrh64R2, AAVrh8. See, e.g., US Patent Publication No. 2007/0036760; US Patent Publication No. 2009/0197338; and EP1310571. See also WO 2003/042397 (AAV7 and other simian AAV), U.S. Pat. Nos. 7,282,199 and 7,790,449 (AAV8). In addition, AAV yet to be discovered, or a recombinant AAV based thereon, may be used as a source for the AAV capsid. These documents also describe other AAV which may be selected for generating AAV and are incorporated by reference. In some embodiments, an AAV capsid for use in the viral vector can be generated by mutagenesis (i.e., by insertions, deletions, or substitutions) of one of the aforementioned AAV capsids or its encoding nucleic acid. In some embodiments, the AAV capsid is chimeric, comprising domains from two or three or four or more of the aforementioned AAV capsid proteins. In some embodiments, the AAV capsid is a mosaic of Vp1, Vp2, and Vp3 monomers from two or three different AAVs or recombinant AAVs. In some embodiments, an rAAV composition comprises more than one of the aforementioned capsids.

### Host Cells Comprising a Recombinant Nucleic Acid Molecule:

In some aspects, provided herein are host cells comprising a recombinant nucleic acid molecule, viral vector, e.g., an AAV vector, or an rAAV disclosed herein. In specific embodiments, the host cells may be suitable for the propagation of AAV.

A vast range of host cells can be used, such as bacteria, yeast, insect, mammalian cells, etc. In some embodiments, the host cell can be a cell (or a cell line) appropriate for production of recombinant AAV (rAAV), for example, a HeLa, Cos-7, HEK293, A549, BHK, Vero, RD, HT-1080, ARPE-19, or MRC-5 cell.

The recombinant nucleic acid molecules or vectors can be delivered into the host cell culture using any suitable method known in the art. In some embodiments, a stable host cell line that has the recombinant nucleic acid molecule or vector inserted into its genome is generated. In some embodiments, a stable host cell line is generated, which contains an rAAV vector described herein. After transfection of the rAAV vector to the host culture, integration of the rAAV into the host genome can be assayed by various methods, such as antibiotic selection, fluorescence-activated cell sorting, southern blot, PCR based detection, fluorescence in situ hybridization as described by Nakai et al, Nature Genetics (2003) 34, 297-302; Philpott et al, Journal of Virology (2002) 76(11):5411-5421, and Howden et al, J Gene Med 2008; 10:42-50. Furthermore, a stable cell line can be established according to protocols well known in the art, such as those described in Clark, Kidney International Vol 61 (2002):S9-S15, and Yuan et al, Human Gene Therapy 2011 May; 22(5):613-24.

### Recombinant AAV for Gene Therapy:

AAV belongs to the family Parvoviridae and the genus Dependovirus. AAV is a small, non-enveloped virus that packages a linear, single-stranded DNA genome. Both sense and antisense strands of AAV DNA are packaged into AAV capsids with equal frequency.

The AAV genome is characterized by two inverted terminal repeats (ITRs) that flank two open reading frames (ORB). In the AAV2 genome, for example, the first 125 nucleotides of the ITR are a palindrome, which folds upon itself to maximize base pairing and forms a T-shaped hairpin structure. The other 20 bases of the ITR, called the D sequence, remain unpaired. The ITRs are cis-acting sequences important for AAV DNA replication; the ITR is the origin of replication and serves as a primer for second-strand synthesis by DNA polymerase. The double-stranded DNA formed during this synthesis, which is called replicating-form monomer, is used for a second round of self-priming replication and forms a replicating-form dimer. These double-stranded intermediates are processed via a strand displacement mechanism, resulting in single-stranded DNA used for packaging and double-stranded DNA used for transcription. Located within the ITR are the Rep binding elements and a terminal resolution site (TRS). These features are used by the viral regulatory protein Rep during AAV replication to process the double-stranded intermediates. In addition to their role in AAV replication, the ITR is also essential for AAV genome packaging, transcription, negative regulation under non-permissive conditions, and site-specific integration (Days and Berns, Cin Microbiol Rev 21(4):583-593, 2008).

The left ORF of AAV contains the Rep gene, which encodes four proteins—Rep78, Rep68, Rep52 and Rep40. The right ORF contains the Cap gene, which produces three viral capsid proteins (VP1, VP2 and VP3). The AAV capsid contains 60 viral capsid proteins arranged into an icosahedral symmetry. VP1, VP2 and VP3 are present in a 1:1:10 molar ratio (Daya and Berns, Clin Microbiol Rev 21(4):583-593, 2008).

AAV is currently one of the most frequently used viruses for gene therapy. Although AAV infects humans and some other primate species, it is not known to cause disease and elicits a very mild immune response. Gene therapy vectors that utilize AAV can infect both dividing and quiescent cells and persist in an extrachromosomal state without integrating into the genome of the host cell. Because of the advantageous features of AAV, the present disclosure contemplates the use of AAV for the recombinant nucleic acid molecules and methods disclosed herein.

AAV possesses several desirable features for a gene therapy vector, including the ability to bind and enter target cells, enter the nucleus, the ability to be expressed in the nucleus for a prolonged period of time, and low toxicity. However, the small size of the AAV genome limits the size of heterologous DNA that can be incorporated. To minimize this problem, AAV vectors have been constructed that do not encode Rep and the integration efficiency element (IEE). The ITRs are retained as they are cis signals required for packaging (Daya and Berns, Clin Microbiol Rev, 21(4):583-593, 2008).

Methods for producing rAAV suitable for gene therapy are well known in the art (see, for example, U.S. Patent Application Nos. 2012/0100606; 2012/0135515; 2011/0229971; and 2013/0072548; and Ghosh et al., Gene Ther 13(4):321-329, 2006), and can be utilized with the recombinant nucleic acid molecules and methods disclosed herein.

In some aspects, the present disclosure provides the use of an rAAV disclosed herein for the treatment of propionic acidemia (PA), wherein the rAAV includes an AAV capsid and a vector genome packaged therein. In some embodiments, the rAAV contains a packaged genome comprising as operably linked components in 5′ to 3′ order: a 5′-ITR, a promoter sequence, a partial or complete coding sequence for PCCA or an isoform thereof, or a functional fragment or functional variant thereof, and a 3′-ITR. In some embodiments, the rAAV contains a packaged genome comprising as operably linked components in 5′ to 3′ order: a 5′-ITR, a promoter sequence, a truncated or complete nucleotide sequence of human PCCA 5′-UTR, a partial or complete coding sequence for PCCA or an isoform thereof, or a functional fragment or functional variant thereof, a truncated or complete nucleotide sequence of human PCCA 3′-UTR, and a 3′-ITR. In an exemplary embodiment, the packaged genome also comprises an enhancer sequence upstream of the promoter sequence, an intron downstream of the promoter, and a polyadenylation sequence upstream of the 3′-ITR. Thus, in another exemplary embodiment, the rAAV contains a packaged genome comprising as operably linked components in 5′ to 3′ order: a 5′-ITR, an enhancer sequence, a promoter sequence, an intron sequence, a partial or complete coding sequence for PCCA or an isoform thereof, or a functional fragment or functional variant thereof, a polyadenylation signal sequence, and a 3′-ITR. In another exemplary embodiment, the rAAV contains a packaged genome comprising as operably linked components in 5′ to 3′ order: a 5′-ITR, an enhancer sequence, a promoter sequence, an intron sequence, a truncated or complete nucleotide sequence of human PCCA 5′-UTR, a partial or complete coding sequence for PCCA or an isoform thereof, or a functional fragment or functional variant thereof, a polyadenylation signal sequence, a truncated or complete nucleotide sequence of human PCCA 3′-UTR, and a 3′-ITR. In a further exemplary embodiment, the rAAV contains a packaged genome comprising as operably linked components in 5′ to 3′ order: an AAV2 5′-ITR sequence, a CMV enhancer, a CBA promoter, an SV40 Small T intron, a coding sequence for PCCA, a BGH polyadenylation signal sequence, and an AAV2 3′-ITR. In a further exemplary embodiment, the rAAV contains a packaged genome comprising as operably linked components in 5′ to 3′ order: an AAV2 5′-ITR sequence, a CMV enhancer, a native PCCA promoter, an SV40 Small T intron, a nucleotide sequence of PCCA 5′-UTR, a coding sequence for PCCA, a native PCCA polyadenylation signal sequence, a nucleotide sequence of PCCA 3′-UTR, and an AAV2 3′-ITR. In some embodiments, the packaged genome further comprises a consensus Kozak sequence located downstream of the intron sequence. In some embodiments, the packaged genome further comprises a consensus Kozak sequence located downstream of the native PCCA 5′-UTR sequence. In some embodiments, the coding sequence for PCCA is selected from SEQ ID NOs: 1-6. In some embodiments, the capsid is an AAV9 capsid.

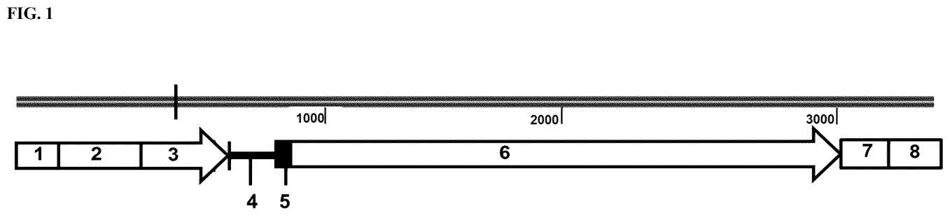

An illustrative diagram showing an exemplary packaged vector genome construct for the expression of PCCA is provided in FIG. 1, which shows in 5′ to 3′ order: a 5′-ITR, a CMV enhancer, a CBA promoter, an SV40 Small T intron, a consensus Kozak sequence, a PCCA coding sequence, an SV40 polyadenylation signal sequence, and a 3′-ITR.

In some aspects, the present disclosure provides the use of an rAAV disclosed herein for the treatment of propionic acidemia (PA), wherein the rAAV includes an AAV capsid and vector genome packaged therein, wherein the packaged genome comprises as operably linked components in 5′ to 3′ order: a 5′-ITR, a promoter sequence, a partial or complete coding sequence for PCCB or an isoform thereof, or a functional fragment or functional variant thereof, and a 3′-ITR. In some embodiments, the rAAV contains a packaged genome comprising as operably linked components in 5′ to 3′ order: a 5′-ITR, a promoter sequence, a truncated or complete nucleotide sequence of human PCCB 5′-UTR, a partial or complete coding sequence for PCCB or an isoform thereof, or a functional fragment or functional variant thereof, a truncated or complete nucleotide sequence of human PCCB 3′-UTR, and a 3′-ITR. In an exemplary embodiment, the packaged genome also comprises an enhancer sequence upstream of the promoter sequence, an intron downstream of the promoter, and a polyadenylation sequence upstream of the 3′-ITR. Thus, in another exemplary embodiment, the rAAV contains a packaged genome comprising as operably linked components in 5′ to 3′ order: a 5′-ITR, an enhancer sequence, a promoter sequence, an intron sequence, a partial or complete coding sequence for PCCB or an isoform thereof, or a functional fragment or functional variant thereof, a polyadenylation signal sequence, and a 3′-ITR. In another exemplary embodiment, the rAAV contains a packaged genome comprising as operably linked components in 5′ to 3′ order: a 5′-ITR, an enhancer sequence, a promoter sequence, an intron sequence, a truncated or complete nucleotide sequence of human PCCB 5′-UTR, a partial or complete coding sequence for PCCB or an isoform thereof, or a functional fragment or functional variant thereof, a polyadenylation signal sequence, a truncated or complete nucleotide sequence of human PCCB 3′-UTR, and a 3′-ITR. In a further exemplary embodiment, the rAAV contains a packaged genome comprising as operably linked components in 5′ to 3′ order: an AAV2 5′-ITR sequence, a CMV enhancer, a CBA promoter, an SV40 Small T intron, a coding sequence for PCCB, a BGH polyadenylation signal sequence, and an AAV2 3′-ITR. In a further exemplary embodiment, the rAAV contains a packaged genome comprising as operably linked components in 5′ to 3′ order: an AAV2 5′-ITR sequence, a CMV enhancer, a native PCCB promoter, an SV40 Small T intron, a nucleotide sequence of PCCB 5′-UTR, a coding sequence for PCCB, a native PCCB polyadenylation signal sequence, a nucleotide sequence of PCCB 3′-UTR, and an AAV2 3′-ITR. In some embodiments, the packaged genome further comprises a consensus Kozak sequence located downstream of the intron sequence. In some embodiments, the packaged genome further comprises a consensus Kozak sequence located downstream of the native PCCB 5′-UTR sequence. In some embodiments, the coding sequence for PCCB is selected from SEQ ID NOs: 7-12. In some embodiments, the capsid is an AAV9 capsid.

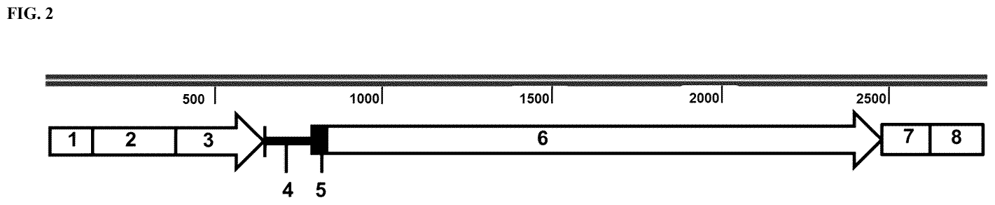

An illustrative diagram showing an exemplary packaged vector genome construct for the expression of PCCB is provided in FIG. 2, which shows in 5′ to 3′ order: a 5′-ITR, a CMV enhancer, a CBA promoter, an SV40 Small T intron, a consensus Kozak sequence, a PCCA coding sequence, an SV40 polyadenylation signal sequence, and a 3′-ITR.

### Protein Localization:

Propionyl-CoA carboxylase is a mutimeric protein that is localized to the mitochondrial matrix (see Browner et al., (1989). Journal of Biological Chemistry. 264:12680-5). Therefore, it is imperative that the gene therapy vectors delivering PCCA and/or PCCB gene(s) are capable of encoding for proteins that are functional and localized in mitochondria. In one aspect, one or more viral vectors disclosed herein drive and regulate the expression of genes (e.g., expression of PCCA or PCCB) that encode for PCCA and/or PCCB protein(s) that localize in the mitochondria.

### Pharmaceutical Compositions:

Compositions comprising the rAAV disclosed herein and a pharmaceutically acceptable carrier are provided by the present disclosure. Suitable pharmaceutical formulations for administration of rAAV can be found, for example, in U.S. Patent Application Publication No. 2012/0219528. The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds, molecules or agents

As highlighted in the preceding paragraph, the present disclosure relates in some aspects to pharmaceutical compositions comprising an rAAV of the invention. In some embodiments, the pharmaceutical composition comprises a pharmaceutically acceptable carrier or excipient. In some embodiments, the pharmaceutical composition is formulated for subcutaneous, intramuscular, intradermal, intraperitoneal, or intravenous administration. In an exemplary embodiment, the pharmaceutical composition is formulated for intravenous administration.

In some embodiments, the rAAV is formulated in a buffer/carrier suitable for infusion in human subjects. The buffer/carrier should include a component that prevents the rAAV from sticking to the infusion tubing but does not interfere with the rAAV binding activity in vivo. Various suitable solutions may include one or more of: a buffering saline, a surfactant, and a physiologically compatible salt or mixture of salts adjusted to an ionic strength equivalent to about 100 mM sodium chloride (NaCl) to about 250 mM sodium chloride, or a physiologically compatible salt adjusted to an equivalent ionic concentration. The pH may be in the range of 6.5 to 8.5, or 7 to 8.5, or 7.5 to 8. A suitable surfactant, or combination of surfactants, may be selected from among Poloxamers, i.e., nonionic triblock copolymers composed of a central hydrophobic chain of polyoxypropylene 10 (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol.

### Methods of Treating Propionic Acidemia:

In yet another aspect, the present disclosure provides methods of treating PA in a human subject comprising administering to the human subject a therapeutically effective amount of at least one rAAV disclosed herein.

In one embodiment, the present disclosure provides a method of treating PA comprising administering an rAAV that includes an AAV capsid and a vector genome packaged therein, wherein the vector genome comprises a partial or complete coding sequence for PCCA or an isoform thereof, or a functional fragment or functional variant thereof.

In another embodiment, the present disclosure provides a method of treating PA comprising administering an rAAV that includes an AAV capsid and a vector genome packaged therein, wherein the vector genome comprises a partial or complete coding sequence for PCCB or an isoform thereof, or a functional fragment or functional variant thereof.

In yet another embodiment, the present disclosure provides a method of treating PA comprising administering (1) an rAAV that includes an AAV capsid and a vector genome packaged therein, wherein the vector genome comprises a partial or complete coding sequence for PCCA or an isoform thereof, or a functional fragment or functional variant thereof; and (2) an rAAV that includes an AAV capsid and a vector genome packaged therein, wherein the vector genome comprises a partial or complete coding sequence for PCCB or an isoform thereof, or a functional fragment or functional variant thereof. In some embodiments, the rAAV of (1) and (2) may administered simultaneously. In some embodiments, the rAAV of (1) and (2) may administered sequentially. In some embodiments, the rAAV of (1) and (2) may administered separately.

In certain embodiments, the present disclosure provides a method of treating PA comprising administering (1) an rAAV that includes an AAV capsid and a vector genome packaged therein in which the coding sequence is for PCCA and not for PCCB, the promoter is a PCCA gene-specific endogenous promoter and not a PCCB gene-specific endogenous promoter, and the polyadenylation signal sequence is a PCCA gene-specific endogenous polyadenylation signal sequence and not a PCCB gene-specific endogenous polyadenylation signal sequence; and/or; and (2) an rAAV that includes an AAV capsid and a vector genome in which the coding sequence is for PCCB and not for PCCA, the promoter is a PCCB gene-specific endogenous promoter and not a PCCA gene-specific endogenous promoter, and the polyadenylation signal sequence is a PCCB gene-specific endogenous polyadenylation signal sequence and not a PCCA gene-specific endogenous polyadenylation signal sequence; and/or. In some embodiments, the rAAV of (1) and (2) may administered simultaneously. In some embodiments, the rAAV of (1) and (2) may administered sequentially. In some embodiments, the rAAV of (1) and (2) may administered separately.

In certain embodiments, the present disclosure provides methods of treating PA in a human subject comprising administering to a human subject diagnosed with at least one mutation in PCCA a therapeutically effective amount of at least one rAAV disclosed herein. In one embodiment, the present disclosure provides a method of treating PA in a human subject diagnosed with at least one mutation in PCCA comprising administering an rAAV that includes an AAV capsid and a vector genome packaged therein, wherein the vector genome comprises a partial or complete coding sequence for PCCA or an isoform thereof, or a functional fragment or functional variant thereof. In certain embodiments, the present disclosure provides a method of treating PA in a human subject diagnosed with at least one mutation in PCCA comprising administering an rAAV that includes an AAV capsid and a vector genome packaged therein in which the coding sequence is for PCCA and not for PCCB, the promoter is a PCCA gene-specific endogenous promoter and not a PCCB gene-specific endogenous promoter, and the polyadenylation signal sequence is a PCCA gene-specific endogenous polyadenylation signal sequence and not a PCCB gene-specific endogenous polyadenylation signal sequence. In certain embodiments, the mutation in PCCA is selected from Table 1. In some embodiments, the coding sequence for PCCA is selected from SEQ ID NOs: 1-6. In some embodiments, the capsid is an AAV9 capsid.

In some aspects, the present disclosure provides methods of treating PA in a human subject comprising administering to a human subject diagnosed with at least one mutation in PCCB a therapeutically effective amount of at least one rAAV disclosed herein. In one embodiment, the present disclosure provides a method of treating PA in a human subject diagnosed with at least one mutation in PCCB comprising administering an rAAV that includes an AAV capsid and a vector genome packaged therein, wherein the vector genome comprises a partial or complete coding sequence for PCCB or an isoform thereof, or a functional fragment or functional variant thereof. In certain embodiments, the present disclosure provides a method of treating PA in a human subject diagnosed with at least one mutation in PCCB comprising administering an rAAV that includes an AAV capsid and a vector genome packaged therein in which the coding sequence is for PCCB and not for PCCA, the promoter is a PCCB gene-specific endogenous promoter and not a PCCA gene-specific endogenous promoter, and the polyadenylation signal sequence is a PCCB gene-specific endogenous polyadenylation signal sequence and not a PCCA gene-specific endogenous polyadenylation signal sequence. In certain embodiments, the mutation in PCCB is selected from Table 2. In some embodiments, the coding sequence for PCCB is selected from SEQ ID NOs: 7-12. In some embodiments, the capsid is an AAV9 capsid.

A review article describing PA-causing mutations in PCCA and PCCB is provided in Ugarte et al., 1999, Hum. Mutat. 14(4): 275-282.

In PA caused by a mutated PCCA gene, the following mutations and polymorphisms in the PCCA gene have been identified:

TABLE 1
PCCA Gene Mutations and Polymorphisms:

| | |
|---|---|
| Missense Mutations: | 148 G→C, 154 C→T, 337 G→A, 416 T→C, 611 T→C, 815 |
| | A→G, 1028 A→G, 1043 T→A, 1061 G→T 1121 G→A, 1193 |
| | C→T, 1601 G→T, 1816 G→C, 1927 G→T |
| Nonsense Mutations: | 862 C→T, 1610 C→G |
| Small Deletions: | 700 del5, 1115del4, 2058del3 |
| Splicing Mutations: | 1645IVS + 1G→A, 1671IVS + 5G→C, 1771IV2-2del9, |
| | 1824IVS + 3del4, 1824IVS + 3insCT |
| Polymorphisms: | 552A→G, 1348A→G |

In PA caused by a mutated PCCB gene, the following mutations and polymorphisms in the PCCB gene have been identified:

TABLE 2
PCCB Gene Mutations and Polymorphisms:

| | |
|---|---|
| Missense Mutations: | 49 C→A, 131 G→C, 318 C→ A, 391 G→C, 493 C→T, 502 |
| | G→A, 593 G→A, 605 T→ A, 683 C→T 1228 C→T, 1283 C→T, |
| | 1325 T→C, 1490 C→T, 1534 C→ T, 1556 T→C, 1606 A→G, |
| Nonsense Mutations: | 1495 C→T, 1593 G→ A |
| Insertions and | 418ins12, 790insG, 1170insT, 1218del14ins12, 1222del3, |
| Deletions: | 1298insA |
| Splicing Mutations: | IVS1 + 3G→C, IVS4 + 3del4, IVS10-11del6, IVS12 + 3del8, |
| | IVS13 + 1G→T |

In some embodiments, the present disclosure provides methods of treating PA in a human subject diagnosed with at least one mutation in PCCA selected from Table 1, comprising administering to said human subject a therapeutically effective amount of at least one rAAV disclosed herein. In one embodiment, the present disclosure provides a method of treating PA in a human subject diagnosed with at least one mutation in PCCA selected from Table 1 comprising administering an rAAV that includes an AAV capsid and a vector genome packaged therein, wherein the vector genome comprises a partial or complete coding sequence for PCCA or an isoform thereof, or a functional fragment or functional variant thereof. In some embodiments, the coding sequence for PCCA is selected from SEQ ID NOs: 1-6. In some embodiments, the capsid is an AAV9 capsid.

In some embodiments, the present disclosure provides methods of treating PA in a human subject diagnosed with at least one mutation in PCCB selected from Table 2, comprising administering to said human subject a therapeutically effective amount of at least one rAAV disclosed herein. In one embodiment, the present disclosure provides a method of treating PA in a human subject diagnosed with at least one mutation in PCCB selected from Table 2 comprising administering an rAAV that includes an AAV capsid and a vector genome packaged therein, wherein the vector genome comprises a partial or complete coding sequence for PCCB or an isoform thereof, or a functional fragment or functional variant thereof. In some embodiments, the coding sequence for PCCB is selected from SEQ ID NOs: 7-12. In some embodiments, the capsid is an AAV9 capsid.

Any suitable method or route can be used to administer an rAAV or an rAAV-containing composition described herein. Routes of administration include, for example, systemic, oral, inhalation, intranasal, intratracheal, intraarterial, intraocular, intravenous, intramuscular, subcutaneous, intradermal, and other parental routes of administration. In some embodiments, the rAAV, a composition comprising an rAAV, or a composition comprising multiple rAAVs (e.g., one rAAV expressing PCCA and a second rAAV expressing PCCB) are administered intravenously.

The specific dose administered can be a uniform dose for each patient, for example, 1.0×10¹¹-1.0×10¹⁴ genome copies (GC) of virus per patient. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can also be adjusted as the progress of the disease is monitored

In some embodiments, the rAAV is administered at a dose of, e.g., about 1.0×10¹¹ genome copies per kilogram of patient body weight (GC/kg) to about 1×10¹⁴ GC/kg, about 5×10¹¹ genome copies per kilogram of patient body weight (GC/kg) to about 5×10¹³ GC/kg, or about 1×10¹² to about 1×10¹³ GC/kg, as measured by qPCR or digital droplet PCR (ddPCR). In some embodiments, the rAAV is administered at a dose of about 1×10¹² to about 1×10¹³ genome copies (GC)/kg. In some embodiments, the rAAV is administered at a dose of about 1.1×10¹¹, about 1.3×10¹¹, about 1.6×10¹¹, about 1.9×10¹¹, about 2×10¹¹, about 2.5×10¹¹, about 3.0×10¹¹, about 3.5×10¹¹, about 4.0×10¹¹, about 4.5×10¹¹, about 5.0×10¹¹, about 5.5×10¹¹, about 6.0×10¹¹, about 6.5×10¹¹, about 7.0×10¹¹, about 7.5×10¹¹, about 8.0×10¹¹, about 8.5×10¹¹, about 9.0×10¹¹, about 9.5×10¹¹, about 1.0×10¹², about 1.5×10¹², about 2.0×10¹², about 2.5×10¹², about 3.0×10¹², about 3.5×10¹², about 4.0×10¹², about 4.5×10¹², about 5.0×10¹², about 5.5×10¹², about 6.0×10¹², about 6.5×10¹², about 7.0×10¹², about 7.5×10¹², about 8.0×10¹², about 8.5×10¹², about 9.0×10¹², about 9.5×10¹², about 1.0×10¹³, about 1.5×10¹³, about 2.0×10¹³, about 2.5×10¹³, about 3.0×10¹³, about 3.5×10¹³, about 4.0×10¹³, about 4.5×10¹³, about 5.0×10¹³, about 5.5×10¹³, about 6.0×10¹³, about 6.5×10¹³, about 7.0×10¹³, about 7.5×10¹³, about 8.0×10¹³, about 8.5×10¹³, about 9.0×10¹³, about 9.5×10¹³ genome copies (GC)/kg. The rAAV can be administered in a single dose, or in multiple doses (such as 2, 3, 4, 5, 6, 7, 8, 9, 10 or more doses) as needed for the desired therapeutic results.

Doses may be given once or more times weekly, monthly or yearly, or even once every 2 to 20 years. For example, each dose may be given at minimum of 1 week apart, 2 weeks apart, 3 weeks apart, a months apart, 3 months apart, 6 months apart, or 1 year apart. Persons of ordinary skill in the art can easily estimate repetition rates for dosing based on measured residence times and concentrations of the targetable construct or complex in bodily fluids or tissues.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the present disclosure, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within the present disclosure, embodiments have been described and depicted in a way that enables a clear and concise disclosure to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a +10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including” is intended merely to illustrate better the present invention and does not pose a limitation on the scope of the invention unless claimed. No language in the specification should be construed as indicating any non-claimed element as essential to the practice of the present invention.

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1: RNA and Protein Expression of PCCA

### RNA Expression

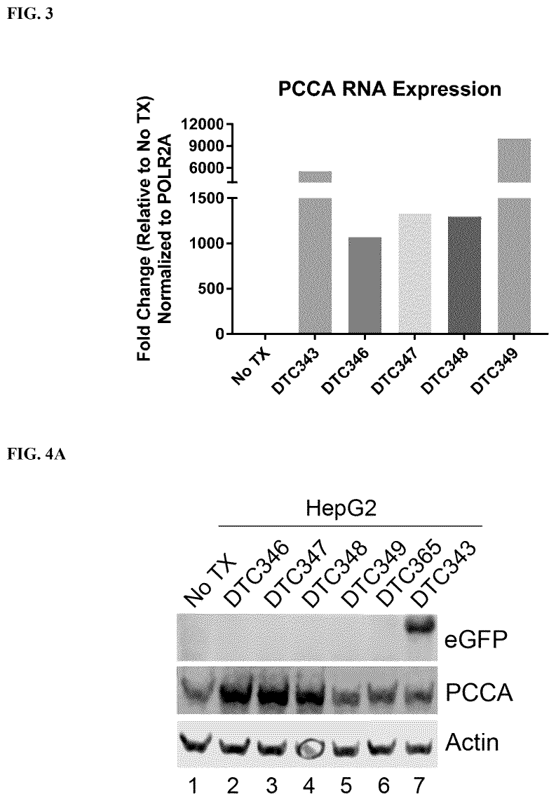

This example relates to RNA expression of PCCA following transfection of continuous hepatocyte cell line (HepG2) as determined by RT-qPCR. Briefly, HepG2 cells were transfected with a control plasmid (plasmid with an empty vector, No TX), an rAAV vector plasmid carrying a nucleotide sequence encoding enhanced green fluorescent protein, eGFP (DTC343), an rAAV vector plasmid carrying a nucleotide sequence encoding the complete native sequence of human PCCA (DTC346), or an rAAV vector plasmid carrying a nucleotide sequence encoding a codon-optimized sequence of human PCCA (DTC347, DTC348, or DTC349) using Lipofectamine 2000 (Invitrogen) according to manufacturer's instructions. HepG2 cells were harvested 72 hours post-transfection and lysed. RNA was extracted from the lysed HepG2 cells using TRIzol reagent (Invitrogen) according to the manufacturer's instructions. The quality and quantity of RNA were measured using NanoDrop ND-1000 (Thermo Scientific) through OD260/280 and OD260/230 ratios. Total RNA was reversed transcribed to cDNA using a High-Capacity cDNA reverse transcription kit (ThermoFisher Scientific) according to the manufacturer's instructions. Real-time RT-qPCR was performed using Power SYBR Green PCR Master Mix (Applied Biosystems) according to the manufacturer's instructions using the Applied Biosystems 7500 Real-Time PCR System. Fold-change in PCCA expression was calculated by the delta-delta Ct (2^{-ΔΔCt}) method utilizing RNA polymerase II polypeptide A (POLR2A) as an internal control for each sample and normalizing the calculated fold-change (see Livak KJ, Schmittgen TD (2001) Analysis of relative gene expression data using real-time quantitative PCR and the 2(−delta delta C(T)) method. Methods 25(4): 402-408) relative to the “no Tx” control. FIG. 3 is a bar graph showing fold-change in PCCA RNA expression as determined by RT-qPCR following transfection of a continuous hepatocyte cell line (HepG2) with a control plasmid (plasmid with an empty vector, no TX), an rAAV vector plasmid carrying a nucleotide sequence encoding enhanced green fluorescent protein, eGFP (DTC343), an rAAV vector plasmid carrying a nucleotide sequence encoding the complete native sequence of human PCCA (DTC346), or an rAAV vector plasmid carrying a nucleotide sequence encoding a codon-optimized sequence of human PCCA (DTC347, DTC348, or DTC349).

The plotted data shows that the RNA expression of complete native human PCCA post-transfection with DTC346 was comparable to expression of two codon-optimized human PCCA versions post-transfection with DTC347 and DTC348. Cells transfected with DTC349 demonstrated higher RNA expression.

### Protein Expression

This example also relates to protein expression of PCCA following transfection of continuous hepatocyte cell lines, HepG2 and Huh7. Briefly, HepG2 and Huh7 cell lines were transfected with a control plasmid (plasmid with an empty vector, No TX), an rAAV vector plasmid carrying a nucleotide sequence encoding the complete native sequence of human PCCA (DTC346), an rAAV vector plasmid carrying a nucleotide sequence encoding a codon-optimized sequence of human PCCA (DTC347, DTC348, or DTC349), an rAAV vector plasmid carrying a nucleotide sequence encoding the complete native sequence of human PCCA and a nucleotide sequence encoding the complete native sequence of human PCCB (DTC365), or an rAAV vector plasmid carrying a nucleotide sequence encoding enhanced green fluorescent protein, eGFP (DTC343) using Lipofectamine 2000 (Invitrogen) according to manufacturer's instructions. Cells were harvested 72 hours post-transfection and lysed with NP-40 lysis buffer (50 mM Tris HCl pH 8.0, 150 mM NaCl, 1.0% NP-40) supplemented with protease inhibitor mix (Sigma) and phosphatase inhibitor cocktail 2+3 (Sigma P5726 and P0044). Proteins were resolved on 10% sodium dodecyl sulfate polyacrylamide electrophoresis (SDS-PAGE) gels and transferred onto IMPMUN-BLOT® polyvinylidene fluoride (PVDF) membrane (Bio-Rad). Western blot analysis was performed using anti-PCCA (Abcam, Cambridge, UK) antibody at 1:1000 dilution followed by a secondary antibody conjugated to far red fluorophores (Licor IRDye Secondary Antibodies).

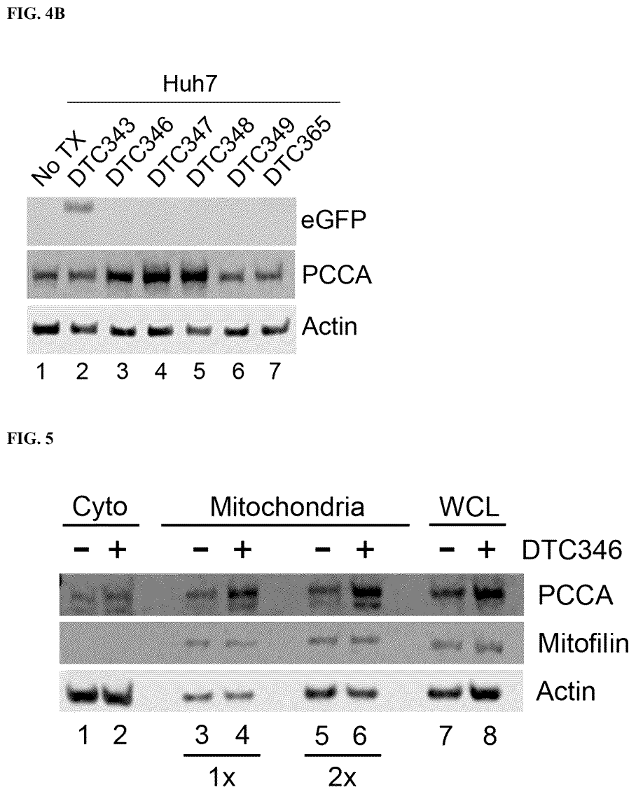

FIG. 4A is an image showing protein expression levels, detected by Western blot, in continuous hepatocyte cell line (HepG2) transfected with a control plasmid (plasmid with an empty vector, no TX), an rAAV vector plasmid carrying a nucleotide sequence encoding the complete native sequence of human PCCA (DTC346), an rAAV vector plasmid carrying a nucleotide sequence encoding a codon-optimized sequence of human PCCA (DTC347, DTC348, or DTC349), an rAAV vector plasmid carrying a nucleotide sequence encoding the complete native sequence of human PCCA and a nucleotide sequence encoding the complete native sequence of human PCCB (DTC365), or an rAAV vector plasmid carrying a nucleotide sequence encoding enhanced green fluorescent protein, eGFP (DTC343), from lane 1 to lane 7, respectively. β-actin (“Actin”) was used as a loading control.

As shown in FIG. 4A, there was an expected basal level of PCCA protein expression in the “no TX” control (lane 1) and overexpression of PCCA protein in cells transfected with DTC346 (lane 2) and in cells transfected with DTC347 (lane 3) or DTC348 (lane 4) when compared to the “no TX” control (lane 1). Cells transfected with DTC349 (lane 5) did not demonstrate overexpression of PCCA protein even though this version was observed to express high amounts of RNA. Cells transfected with DTC343 (lane 7) demonstrated eGFP expression as expected.

FIG. 4B is an image showing protein expression levels, detected by Western blot, in continuous hepatocyte cell line (Huh7) transfected with a control plasmid (plasmid with an empty vector, no TX), an rAAV vector plasmid carrying a nucleotide sequence encoding enhanced green fluorescent protein, eGFP (DTC343), an rAAV vector plasmid carrying a nucleotide sequence encoding the complete native sequence of human PCCA (DTC346), an rAAV vector plasmid carrying a nucleotide sequence encoding a codon-optimized sequence of human PCCA (DTC347, DTC348, or DTC349), or an rAAV vector plasmid carrying a nucleotide sequence encoding the complete native sequence of human PCCA and a nucleotide sequence encoding the complete native sequence of human PCCB (DTC365), from lane 1 to lane 7, respectively. β-actin (“Actin”) was used as a loading control.

As shown in FIG. 4B, there was an expected basal level of PCCA protein expression in the “no TX” control (lane 1) and overexpression of PCCA protein in cells transfected with DTC346 (lane 3) and in cells transfected with DTC347 (lane 4) or DTC348 (lane 5) when compared to the “no TX” control (lane 1). Cells transfected with DTC349 (lane 6) did not demonstrate overexpression of PCCA protein even though this version was observed to express high amounts of RNA. Cells transfected with DTC343 (lane 2) demonstrated eGFP expression as expected.

### Example 2: Overexpressed Human PCCA Protein Localizes to Mitochondria

This example shows that human PCCA overexpressed in continuous hepatocyte cell line (HepG2) localizes to mitochondria. Briefly, continuous hepatocyte cell line (HepG2) was transfected with an rAAV vector plasmid carrying a nucleotide sequence encoding the complete native sequence of human PCCA (DTC346). Following transfection, cells were subjected to subcellular fractionation where cytoplasmic and mitochondrial fractions were isolated. Whole cell lysates were also processed. Proteins were resolved on 10% sodium dodecyl sulfate polyacrylamide electrophoresis (SDS-PAGE) gels and transferred onto IMMUN-BLOT® polyvinylidene fluoride (PVDF) membrane (Bio-Rad). Western blot analysis was performed using anti-PCCA (Abcam, Cambridge, UK) antibody at 1:1000 dilution followed by a secondary antibody conjugated to far red fluorophores (Licor IRDye Secondary Antibodies). FIG. 5 is an image showing PCCA protein expression levels, detected by Western blot, in whole cell lysates (WCL); subcellular fractions (cytoplasmic fractions (cyto); and mitochondrial fractions (mitochondria)), isolated from a continuous hepatocyte cell line (HepG2) transfected with or without an rAAV vector plasmid carrying a nucleotide sequence encoding the complete native sequence of human PCCA (DTC346). 1× and 2× denote relative volumes of loaded mitochondrial fractions. β-actin (“Actin”) was used as a loading control.

As shown in FIG. 5, and consistent with results shown in FIGS. 4A-B, cells transfected with rAAV vector plasmid carrying a nucleotide sequence encoding the complete native sequence of human PCCA (DTC346) demonstrated PCCA overexpression when compared to untransfected control cells (lane 7 versus lane 8 in FIG. 5) in whole cell lysates. Levels of mitofilin, a protein known to be localized to mitochondria, were interrogated to determine the relative purity of isolated cellular fractions. As shown in FIG. 5, cytoplasmic fractions for untransfected and DTC346-transfected cells did not demonstrate detectable levels of mitofilin (lane 1 versus lane 2 in FIG. 5), whereas mitofilin was readily detected in the mitochondrial fractions and whole cell lysates as expected (lanes 3-8 in FIG. 5). Overexpressed PCCA localized predominantly to mitochondria (lane 2 versus lanes 4 and 6 in FIG. 5).

### Example 3: Comparison of PCCA AAV9 Titer Yield

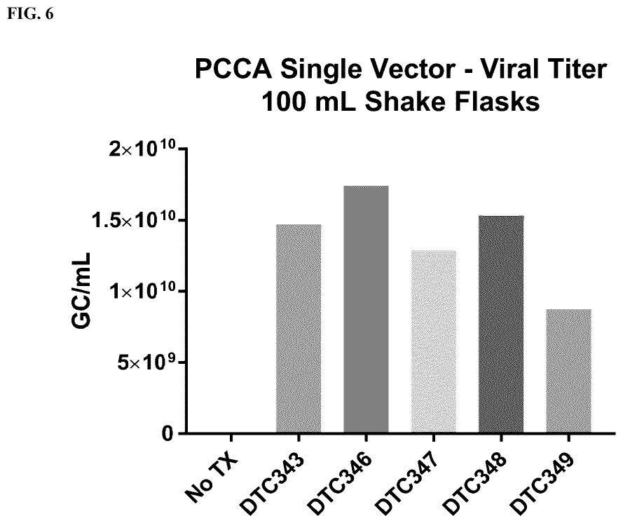

This example relates to comparison of PCCA AAV9 titer yield from rAAV vector plasmid carrying a nucleotide sequence encoding the complete native sequence of human PCCA (DTC346) or an rAAV vector plasmid carrying a nucleotide sequence encoding a codon-optimized sequence of human PCCA (DTC347, DTC348, or DTC349). A continuous kidney cell line (HEK293) was triple transfected with an rAAV vector plasmid carrying a nucleotide sequence encoding the complete native sequence of human PCCA (DTC346), an rAAV vector plasmid carrying a nucleotide sequence encoding a codon-optimized sequence of human PCCA (DTC347, DTC348, or DTC349), or an rAAV vector plasmid carrying a nucleotide sequence encoding enhanced green fluorescent protein, eGFP (DTC343); each were expressed in a cell co-transfected with an AAV9 capsid expressing plasmid (pAAV2/9) and a plasmid supplying adenovirus helper functions (pAdHelper). Untransfected cells were used as control and are represented as “no TX” in FIG. 6. Cell supernatant was harvested and treated with DNase I, or equivalent, to eliminate non-encapsidated DNA and facilitate quantification of DNase-resistant particle (DRP) titer utilizing a primer/probe set specific to the polyadenylation signal included in the design of these constructs. FIG. 6 is a bar graph showing, from left-to-right, fold-change of PCCA AAV9 titer (in genome copies (GC)/mL) as determined by qPCR following triple transfection of a continuous kidney cell line (HEK293) with an rAAV vector plasmid carrying a nucleotide sequence encoding enhanced green fluorescent protein, eGFP (DTC343); an rAAV vector plasmid carrying a nucleotide sequence encoding the complete native sequence of human PCCA (DTC346); or an rAAV vector plasmid carrying a nucleotide sequence encoding a codon-optimized sequence of human PCCA (DTC347, DTC348, or DTC349); each are expressed in a cell co-transfected with an AAV9 capsid-expressing plasmid (pAAV2/9) and a plasmid supplying adenovirus helper functions (pAdHelper). Untransfected cells were used as control and are represented as “no TX”. FIG. 6 shows fold-change in PCCA AAV9 titer (in genome copies (GC)/ml) relative to untransfected cells as determined by qPCR. The data shown in FIG. 6 demonstrates comparable rAAV titers between the native complete human PCCA expressing construct (DTC346) and all codon-optimized versions (DTC347, DTC348, and DTC349).

### Example 4: Testing PCCA DNA Integrity

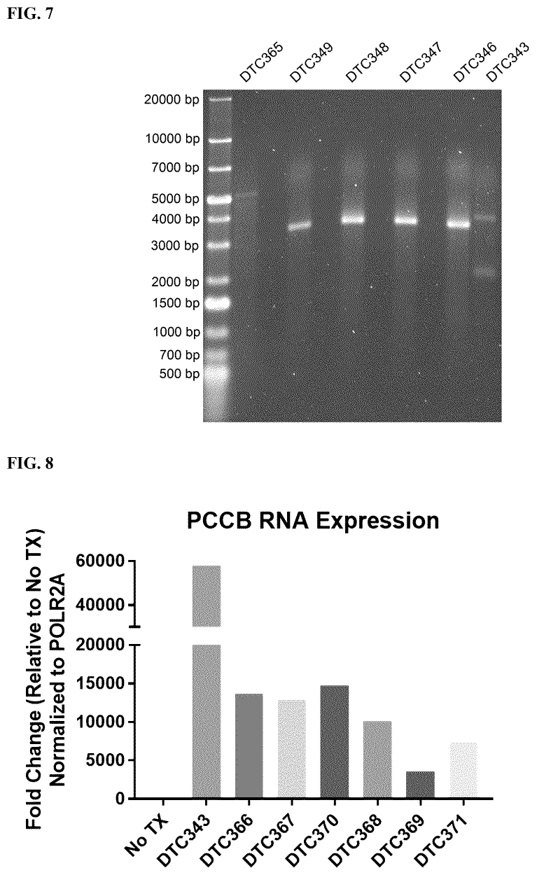

Following generation of AAV9 rAAV viral particles via triple transfection in HEK293 cells, cellular supernatant was harvested and incubated with AAVX resin (Thermo Fisher), or equivalent. AAV9 rAAV viral particles were purified and then concentrated using Amicon centrifugal filters (Millipore Sigma). Purified virus was incubated in an alkaline lysis buffer, loaded into a DNA agarose gel and run overnight in the presence of an alkaline running buffer. FIG. 7 is an image of a DNA alkaline agarose gel of affinity purified AAV9 particles containing PCCA and PCCB-expressing recombinant AAV (rAAV) vector cassette (DTC365), PCCA-expressing recombinant AAV (rAAV) vector cassettes (DTC349, DTC348, DTC347, or DTC346), or eGFP-expressing recombinant AAV (rAAV) vector cassette (DTC343), from left to right, respectively.

As shown in FIG. 7, the gel image shows clear single bands at the expected size for native and codon-optimized PCCA vector cassettes suggesting appropriate packaging of vector genome (DTC346, DTC347, DTC348, and DTC349). The eGFP control (DTC343) demonstrated packaging of two bands, consistent with known limitations of packaging small vector genomes.

### Example 5: RNA and Protein Expression of PCCB

### RNA Expression

This example relates to RNA expression of PCCB following transfection of continuous hepatocyte cell line (HepG2) as determined by RT-qPCR. Briefly, HepG2 cells were transfected with a control plasmid (plasmid with an empty vector, No TX), an rAAV vector plasmid carrying a nucleotide sequence encoding enhanced green fluorescent protein, eGFP (DTC343), an rAAV vector plasmid carrying a nucleotide sequence encoding the complete native sequence of human PCCB (DTC366), or an rAAV vector plasmid carrying a nucleotide sequence encoding a codon-optimized sequence of human PCCB (DTC367, DTC370, DTC368, DTC369, or DTC371) using Lipofectamine 2000 (Invitrogen) according to manufacturer's instructions. HepG2 cells were harvested 72 hours post-transfection and lysed. RNA was extracted from the lysed HepG2 cells using TRIzol reagent (Invitrogen) according to the manufacturer's instructions. The quality and quantity of RNA were measured using NanoDrop ND-1000 (Thermo Scientific) through OD260/280 and OD260/230 ratios. Total RNA was reversed transcribed to cDNA using a High-Capacity cDNA reverse transcription kit (ThermoFisher Scientific) according to the manufacturer's instructions. Real-time RT-qPCR was performed using Power SYBR Green PCR Master Mix (Applied Biosystems) according to the manufacturer's instructions using the Applied Biosystems 7500 Real-Time PCR System. Fold-change in PCCB expression was calculated by the delta-delta Ct (2^{-ΔΔCt}) method utilizing RNA polymerase II polypeptide A (POLR2A) as an internal control for each sample and normalizing the calculated fold-change (see Livak K J, Schmittgen T D (2001) Analysis of relative gene expression data using real-time quantitative PCR and the 2(−delta delta C(T)) method. Methods 25(4): 402-408) relative to the “No Tx” control. FIG. 8 is a bar graph showing fold-change in PCCB RNA expression as determined by RT-qPCR following transfection of a continuous hepatocyte cell line (HepG2) with a control plasmid (plasmid with an empty vector, no TX), an rAAV vector plasmid carrying a nucleotide sequence encoding enhanced green fluorescent protein, eGFP (DTC343), an rAAV vector plasmid carrying a nucleotide sequence encoding the complete native sequence of human PCCB (DTC366), or an rAAV vector plasmid carrying a nucleotide sequence encoding a codon-optimized sequence of human PCCB (DTC367, DTC370, DTC368, DTC369, or DTC371).

The plotted data shows that the RNA expression of complete native sequence of human PCCB post-transfection with DTC366 was comparable to expression of four codon-optimized human PCCB versions post-transfection with DTC367, DTC368, DTC370, or DTC371. Cells transfected with DTC369 demonstrated lower RNA expression.

### Protein Expression

This example also relates to protein expression of PCCB following transfection of continuous hepatocyte cell lines, HepG2 and Huh7. Briefly, HepG2 and Huh7 cell lines were transfected with a control plasmid (plasmid with an empty vector, no TX), an rAAV vector plasmid carrying a nucleotide sequence encoding enhanced green fluorescent protein, eGFP (DTC343), an rAAV vector plasmid carrying a nucleotide sequence encoding the complete native sequence of human PCCB (DTC366), or an rAAV vector plasmid carrying a nucleotide sequence encoding a codon-optimized sequence of human PCCB (DTC367, DTC368, DTC369, DTC370, or DTC371) using Lipofectamine 2000 (Invitrogen) according to manufacturer's instructions. Cells were harvested 72 hours post-transfection and lysed with NP-40 lysis buffer (50 mM Tris HCl pH 8.0, 150 mM NaCl, 1.0% NP-40) supplemented with protease inhibitor mix (Sigma) and phosphatase inhibitor cocktail 2+3 (Sigma P5726 and P0044). Proteins were resolved on 10% sodium dodecyl sulfate polyacrylamide electrophoresis (SDS-PAGE) gels and transferred onto IMPMUN-BLOT® polyvinylidene fluoride (PVDF) membrane (Bio-Rad). Western blot analysis was performed using an anti-PCCB (Abcam, Cambridge, UK) antibody at 1:1000 dilution followed by a secondary antibody conjugated to far red fluorophores (Licor IRDye Secondary Antibodies).

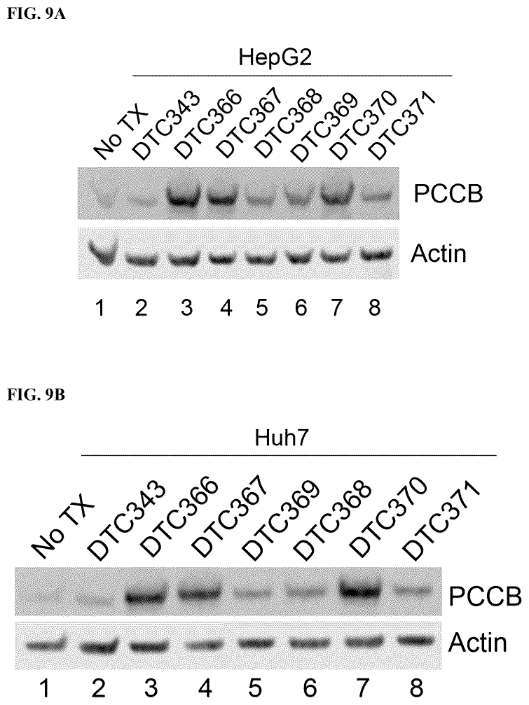

FIG. 9A is an image showing protein expression levels, detected by Western blot, in continuous hepatocyte cell line (HepG2) transfected with a control plasmid (plasmid with an empty vector, no TX), an rAAV vector plasmid carrying a nucleotide sequence encoding enhanced green fluorescent protein, eGFP (DTC343), an rAAV vector plasmid carrying a nucleotide sequence encoding the complete native sequence of human PCCB (DTC366), or an rAAV vector plasmid carrying a nucleotide sequence encoding a codon-optimized sequence of human PCCB (DTC367, DTC368, DTC369, DTC370, or DTC371), from lane 1 to lane 8, respectively. β-actin (“Actin”) was used as a loading control.

As shown in FIG. 9A, there was an expected basal level of PCCB protein expression in the “no TX” control (lane 1) and overexpression of PCCB protein in cells transfected with DTC366 (lane 3) and in cells transfected with DTC367 (lane 4) or DTC370, (lane 7) when compared to the “no TX” control (lane 1). Cells transfected with DTC368 (lane 5), DTC369 (lane 6), or DTC371 (lane 8) did not demonstrate overexpression of PCCB protein. Cells transfected with DTC343 (lane 2) demonstrated eGFP expression as expected.

FIG. 9B is an image showing protein expression levels, detected by Western blot, in continuous hepatocyte cell line (Huh7) transfected with a control plasmid (plasmid with an empty vector, no TX), an rAAV vector plasmid carrying a nucleotide sequence encoding enhanced green fluorescent protein, eGFP (DTC343), an rAAV vector plasmid carrying a nucleotide sequence encoding the complete native sequence of human PCCB (DTC366), or an rAAV vector plasmid carrying a nucleotide sequence encoding a codon-optimized sequence of human PCCB (DTC367, DTC369, DTC368, DTC370, or DTC371), from lane 1 to lane 8, respectively. β-actin (“Actin”) was used as a loading control.

As shown in FIG. 9B, there was an expected basal level of PCCB protein expression in the “no TX” control (lane 1) and overexpression of PCCB protein in cells transfected with DTC366 (lane 3) and in cells transfected with DTC367 (lane 4) or DTC370 (lane 7) when compared to the “no TX” control (lane 1). Cells transfected with DTC368 (lane 6), DTC369 (lane 5), or DTC371 (lane 8) did not demonstrate overexpression of PCCB protein. Cells transfected with DTC343 (lane 2) demonstrated eGFP expression as expected.

### Example 6: Overexpressed Human PCCB Protein Localizes to Mitochondria

This example shows that human PCCB overexpressed in continuous hepatocyte cell line (HepG2) localizes to mitochondria. Briefly, continuous hepatocyte cell line (HepG2) was transfected with an rAAV vector plasmid carrying a nucleotide sequence encoding the complete native sequence of human PCCB (DTC366). Following transfection, cells were subjected to subcellular fractionation where cytoplasmic and mitochondrial fractions were isolated. Whole cell lysates were also processed. Proteins were resolved on 10% sodium dodecyl sulfate polyacrylamide electrophoresis (SDS-PAGE) gels and transferred onto IMMUN-BLOT® polyvinylidene fluoride (PVDF) membrane (Bio-Rad). Western blot analysis was performed using anti-PCCB (Abcam, Cambridge, UK) antibody at 1:1000 dilution followed by a secondary antibody conjugated to far red fluorophores (Licor IRDye Secondary Antibodies).

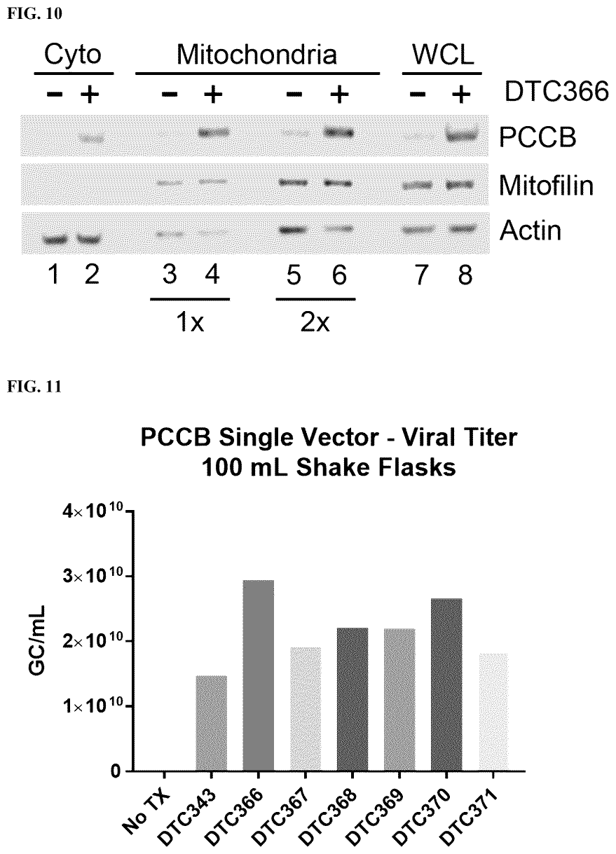

FIG. 10 is an image showing PCCB protein expression levels, detected by Western blot, in whole cell lysates (WCL); subcellular fractions (cytoplasmic fractions (cyto); and mitochondrial fractions (mitochondria)), isolated from a continuous hepatocyte cell line (HepG2) transfected with or without an rAAV vector plasmid carrying a nucleotide sequence encoding the complete native sequence of human PCCB (DTC366). 1× and 2× denote relative volumes of loaded mitochondrial fractions. β-actin (“Actin”) was used as a loading control.

As shown in FIG. 10, and consistent with previous results, cells transfected with rAAV vector plasmid carrying a nucleotide sequence for encoding the complete native sequence of human PCCB (DTC366) demonstrated PCCB protein overexpression when compared to untransfected control cells (lane 7 versus lane 8) in whole cell lysates. Levels of mitofilin, a protein known to be localized to mitochondria, were interrogated to determine the relative purity of isolated cellular fractions. As shown in FIG. 10, cytoplasmic fractions for untransfected and DTC366-transfected cells did not demonstrate detectable levels of mitofilin (lane 1 versus lane 2), whereas mitofilin was readily detected in the mitochondrial fractions and whole cell lysates as expected (lanes 3-8). Overexpressed PCCB localized predominantly to mitochondria (lane 2 versus lanes 4 and 6).

### Example 7: Comparison of PCCB AAV9 Titer Yield

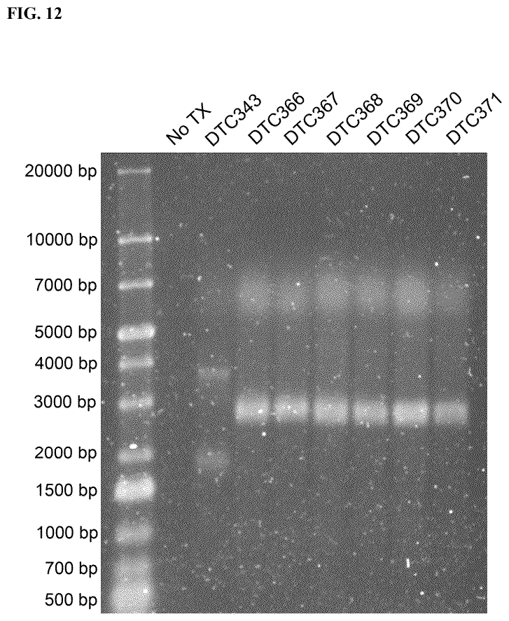

This example relates to comparison of PCCB AAV9 titer yield from rAAV vector plasmid carrying a nucleotide sequence encoding the complete native sequence of human PCCB (DTC366) or an rAAV vector plasmid carrying a nucleotide sequence encoding a codon-optimized sequence of human PCCB (DTC367, DTC368, DTC369, DTC370, or DTC371). A continuous kidney cell line (HEK293) was triple transfected with an rAAV vector plasmid carrying a nucleotide sequence encoding enhanced green fluorescent protein, eGFP (DTC343); an rAAV vector plasmid carrying a nucleotide sequence encoding the complete native sequence of human PCCB (DTC366); or an rAAV vector plasmid carrying a nucleotide sequence encoding a codon-optimized sequence of human PCCA (DTC367, DTC368, DTC369, DTC370, or DTC371); each were expressed in a cell co-transfected with an AAV9 capsid expressing plasmid (pAAV2/9) and a plasmid supplying adenovirus helper functions (pAdHelper). Untransfected cells were used as control and are represented as “no TX” in FIG. 11. Cell supernatant was harvested and treated with DNase I, or equivalent, to eliminate non-encapsidated DNA and facilitate quantification of DNase-resistant particle (DRP) titer utilizing a primer/probe set specific to the polyadenylation signal included in the design of these constructs. FIG. 11 is a bar graph showing, from left-to-right, fold-change of PCCB AAV9 titer (in genome copies (GC)/mL) as determined by qPCR following triple transfection of a continuous kidney cell line (HEK293) with an rAAV vector plasmid carrying nucleotide sequence encoding enhanced green fluorescent protein, eGFP (DTC343); an rAAV vector plasmid carrying nucleotide sequence encoding the complete native sequence of human PCCB (DTC366); or an rAAV vector plasmid carrying a nucleotide sequence encoding a codon-optimized sequence of human PCCA (DTC367, DTC368, DTC369, DTC370, or DTC371); each are expressed in a cell co-transfected with an AAV9 capsid expressing plasmid (pAAV2/9) and a plasmid supplying adenovirus helper functions (pAdHelper). Untransfected cells were used as control and are represented as “no TX”. FIG. 11 shows fold-change in PCCB AAV9 titer (in genome copies (GC)/mL) relative to untransfected cells as determined by qPCR. The data shown in FIG. 11 demonstrates comparable rAAV titers between the native complete human PCCB expressing construct (DTC366) and all codon-optimized versions (DTC367, DTC368, DTC369, DTC370, and DTC371).

### Example 8: Testing PCCB DNA Integrity

Following generation of AAV9 rAAV viral particles via triple transfection in HEK293 cells, cellular supernatant was harvested and incubated with AAVX resin (Thermo Fisher), or equivalent. AAV9 rAAV viral particles were purified and then concentrated using Amicon centrifugal filters (Millipore Sigma). Purified virus was incubated in an alkaline lysis buffer, loaded into a DNA agarose gel and run overnight in the presence of an alkaline running buffer. FIG. 12 is an image of a DNA alkaline agarose gel of affinity purified AAV9 particles containing control plasmid (plasmid with an empty vector, no TX), an eGFP-expressing recombinant AAV (rAAV) vector cassette (DTC343), or a PCCB-expressing recombinant AAV (rAAV) vector cassette (DTC366, DTC367, DTC368, DTC369, DTC370, or DTC371), from left to right, respectively.

As shown in FIG. 12, the gel image shows clear single bands at the expected size for native and codon-optimized PCCB vector cassettes suggesting appropriate packaging of vector genome (DTC366, DTC367, DTC368, DTC369, DTC370, and DTC371). The eGFP control (DTC343) demonstrated packaging of two bands, consistent with known limitations of packaging small vector genomes.

### Example 9: Administration of PCCA Viral Vectors to Wild-Type Mice

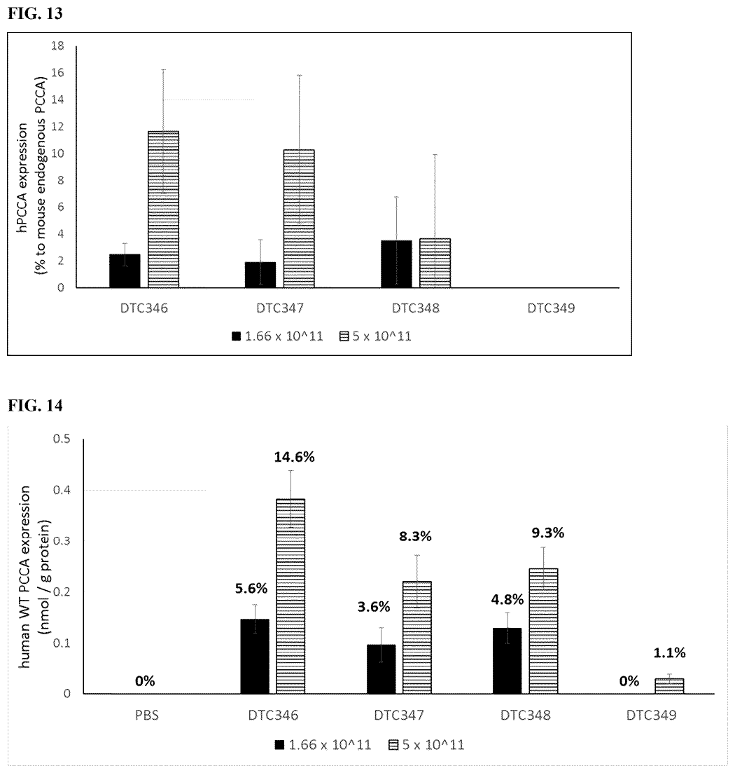

This example relates to human PCCA protein expression in wild-type FVB mice following treatment with rAAV expressing the complete native human PCCA (DTC346) or a codon-optimized (DTC347, DTC348, and DTC349) human PCCA. Briefly, mice (n=2 or 3) were intravenously injected with 1.66×10¹¹ viral genomes (VGs) or 5×10¹¹ VGs of an rAAV expressing the complete native human PCCA (DTC346) represented by SEQ ID NO: 38 or an rAAV expressing a codon-optimized human PCCA (DTC347, DTC348, and DTC349). Human PCCA protein and endogenous mouse PCCA protein were detected by LC-MS. FIG. 13 is a bar graph showing the percentage of human PCCA protein expression relative to mouse endogenous PCCA expression in wild-type FVB mice following treatment with rAAV encoding the complete native sequence of human PCCA (DTC346), or a codon-optimized sequence of human PCCA (DTC347, DTC348, or DTC349). Error bars represent standard deviations. Data presented in FIG. 13 shows that human PCCA expressed from administration of 1.66×10¹¹ VGs of native PCCA (DTC346) was 2.5% to endogenous mouse PCCA levels and human PCCA expressed from administration of 5×10¹¹ VGs of native PCCA viral vector (DTC346) was 11.6% to endogenous mouse PCCA levels.

### Example 10: Human PCCA Activity in Hypomorphic PCCA Mouse Model

This example relates to testing human PCCA activity in a hypomorphic PCCA mouse model. Deletion of the PCCA gene in mice mimics the most severe forms of the human disease. Mice with PCCA gene deletion die within 36 hours of birth, making it difficult to test intravenous systemic therapies in them (see Guenzel et al., Mol Ther. 2013 July; 21(7): 1316-1323). Hypomorphic PCCA mice have reduced PCCA activity due to an introduction of the transgene coding human PCCA with pathogenic A138T mutation on the background of mouse PCCA knockout. This mutant PCCA gene results in elevated levels of propionylcarnitine, methylcitrate, glycine, alanine, lysine, ammonia, and markers associated with cardiomyopathy, similar to levels of these compounds in patients with propionic acidemia (PA). Briefly, mice (n=5) were intravenously injected with 1.66×10¹¹ viral genomes (VGs) or 5×10¹¹ VGs of an rAAV encoding the complete native sequence of human PCCA (DTC346) represented by SEQ ID NO: 38. PBS-treated mice were used as control mice.

### PCCA Expression Level and Enzymatic Activity Assay

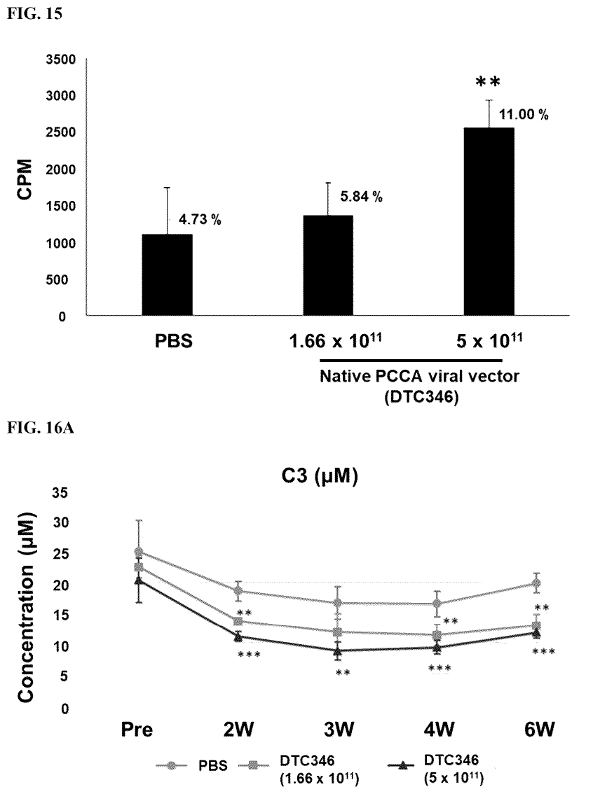

Six weeks after injection, mouse livers were homogenized and human wild-type PCCA protein was measured by LC-MS. FIG. 14 is a bar graph showing the concentration (in nmol/g protein) of human wild-type PCCA protein expressed in a hypomorphic PCCA mouse model (A138T mutant) following treatment with rAAV encoding the complete native sequence of human PCCA (DTC346) or a codon-optimized sequence of human PCCA (DTC347, DTC348, or DTC349). The listed percentage number denotes human wildtype PCCA expression calculated as a percentage to endogenous PCCA protein level of wildtype FVB mice. Error bars represent standard deviations. The listed percentage number denotes human wild-type PCCA expression calculated as a percentage to endogenous PCCA protein level of wild-type FVB mice. Consistent with previous results, human PCCA protein expressed from rAAV encoding a codon-optimized human PCCA demonstrated lower expression levels when compared to native human PCCA protein expressed from rAAV encoding the complete native human PCCA. PCCA enzyme activity of the liver homogenates was measured as incorporation of sodium bicarbonate [C14] using a scintillation counter. FIG. 15 is a bar graph of human PCCA activity in a hypomorphic PCCA mouse model (A138T mutant) following treatment with rAAV encoding the complete native sequence of human PCCA (DTC346), as measured by mean counts per minute (CPM) data. Error bars represent standard deviations. The listed percentage numbers describe the CPM data relative to those observed in wild-type FVB mice. ** denotes p<0.01 in comparison to PBS-treated group using Dunnett multiple comparison test.

As shown in FIG. 15, mice injected with the higher dose (5×10¹¹ VGs) of an rAAV encoding the complete native human PCCA demonstrated a statistically significant improvement in PCCA activity (11%) compared to PBS-treated mice.

### Effect of Administering Native PCCA Viral Vector to Hypomorphic PCCA Mice on Concentration of Known Biomarkers of Propionic Acidemia (PA)

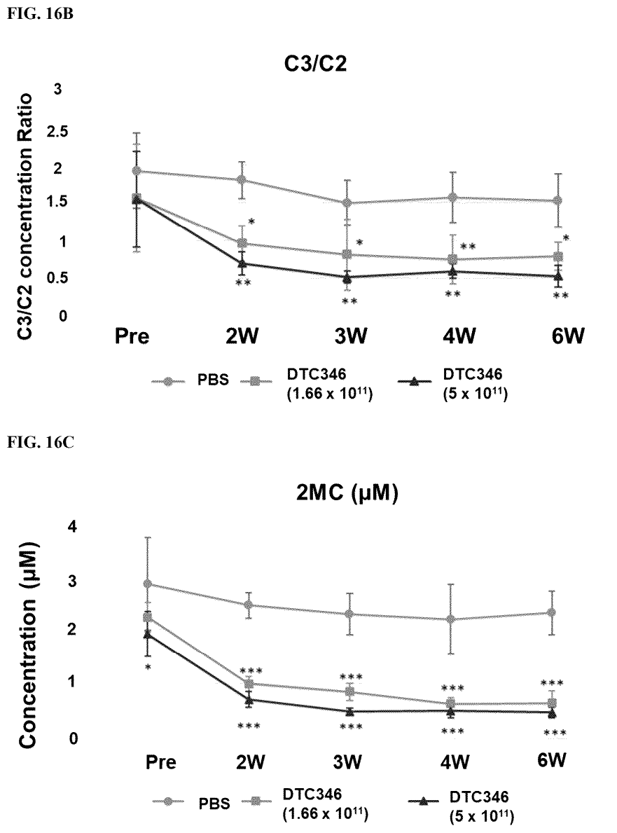

This example also relates to testing the effect of administering rAAV encoding the complete native sequence of human PCCA (DTC346) to hypomorphic PCCA mice on plasma concentration of known biomarkers of PA. Briefly, mice (n=5) were intravenously injected with 1.66×10¹¹ VGs or 5×10¹¹ VGs of an rAAV encoding the complete native sequence of human PCCA (DTC346) represented by SEQ ID NO: 38. Plasma concentrations of known biomarkers of PA (propionylcarnitine (C3), acetylcarnitine (C2), and 2-methylcitrate (2MC)) were determined by liquid chromatography-mass spectrometry (LC-MS). FIGS. 16A-C are bar graphs showing plasma concentrations of known biomarkers of propionic acidemia in a hypomorphic PCCA mouse model (A138T mutant) before (Pre) and 2, 3, 4, and 6 weeks (2W, 3W, 4W, and 6W, respectively) after treatment with rAAV encoding the complete native sequence of human PCCA (DTC346). Error bars represent standard deviations. ** denotes p<0.01 in comparison to PBS-treated group and *** denotes p<0.001 in comparison to PBS-treated group using Dunnett multiple comparison test. FIG. 16A is a bar graph showing plasma concentrations of C3 (propionylcarnitine) in a hypomorphic PCCA mouse model (A138T mutant) following treatment with rAAV encoding the complete native sequence of human PCCA (DTC346). FIG. 16B is bar graph of plasma C3/C2 concentration ratio (propionylcarnitine/acetylcarnitine) in a hypomorphic PCCA mouse model (A138T mutant) following treatment with rAAV encoding the complete native sequence of human PCCA (DTC346). FIG. 16C is a bar graph showing plasma concentrations of 2-methylcitrate (2MC) in a hypomorphic PCCA mouse model (A138T mutant) following treatment with rAAV encoding the complete native sequence of human PCCA (DTC346).

Mice treated with either doses (1.66×10¹¹ VGs or 5×10¹¹ VGs) of rAAV encoding the complete native sequence of human PCCA (DTC346) demonstrated statistically significant reductions in plasma C3, C3/C2 and 2-MC levels compared to PBS-treated mice.

### NUMBERED EMBODIMENTS

Embodiments disclosed herein include embodiments P1 to P136 as provided in the numbered embodiments of the disclosure.

Embodiment P1: A recombinant adeno-associated virus (rAAV), said rAAV comprising an AAV capsid, and a vector genome packaged therein, said vector genome comprising in 5′ to 3′ order:
(a) a 5′ ITR sequence;
(b) a promoter sequence;
(c) a partial or complete coding sequence for PCCA; and
(d) a 3′ ITR sequence.

Embodiment P2: The rAAV according to embodiment P1, wherein the AAV capsid is from an AAV of serotype 1, 2, 3, 4, 5, 6, 7, 8, 9, 10, 11, 12, rh10, or hu37.

Embodiment P3: The rAAV according to embodiment P2, wherein the AAV capsid is from AAV9.

Embodiment P4: The rAAV according to embodiment P2, wherein the AAV capsid is from AAV8.

Embodiment P5: The rAAV according to embodiment P1, wherein the AAV capsid is an AAV9 variant capsid.

Embodiment P6: The rAAV according to any of embodiments P1 to P5, wherein the promoter is selected from a chicken β-actin (CBA) promoter, a cytomegalovirus immediate early gene (CMV) promoter, a transthyretin (TTR) promoter, a thyroxine binding globulin (TBG) promoter, an alpha-1 anti-trypsin (A1AT) promoter, and a CAG promoter.

Embodiment P7: The rAAV according to embodiment P6, wherein the promoter is the CBA promoter.

Embodiment P8: The rAAV according to any of embodiments P1 to P7, wherein the partial or complete coding sequence for PCCA is a wild-type coding sequence.

Embodiment P9: The rAAV according to embodiment P8, wherein the coding sequence for PCCA comprises SEQ ID NO: 1.

Embodiment P10: The rAAV according to any of embodiments P1 to P7, wherein the partial or complete coding sequence for PCCA is a codon-optimized coding sequence.

Embodiment P11: The rAAV according to embodiment P10, wherein the coding sequence for PCCA comprises a coding sequence selected from SEQ ID NOs: 2-6.

Embodiment P12: The rAAV according to any of embodiments P1 to P11, wherein the 5′ ITR sequence is from AAV2.

Embodiment P13: The rAAV according to any of embodiments P1 to P11, wherein the 3′ ITR sequence is from AAV2.

Embodiment P14: The rAAV according to any of embodiments P1 to P11, wherein the 5′ ITR sequence and the 3′ ITR sequence are from AAV2.

Embodiment P15: The rAAV according to any of embodiments P12 to P14, wherein the 5′ ITR sequence and the 3′ ITR sequence comprises or consists of SEQ ID NO: 15.

Embodiment P16: The rAAV according to any of embodiments P1 to P11, wherein the 5′ ITR sequence and/or the 3′ ITR sequence are from a non-AAV2 source.

Embodiment P17: The rAAV according to any of embodiments P1 to P16, wherein the packaged genome further comprises one or more enhancer sequences.

Embodiment P18: The rAAV according to embodiment P17, wherein the enhancer is selected from a cytomegalovirus immediate early gene (CMV) enhancer, a transthyretin enhancer (enTTR), a chicken β-actin (CBA) enhancer, an En34 enhancer, and an ApoE enhancer.

Embodiment P19: The rAAV according to embodiment P18, wherein the enhancer is the CMV enhancer.

Embodiment P20: The rAAV according to embodiment P19, wherein the enhancer comprises or consists of SEQ ID NO: 19.

Embodiment P21: The rAAV according to embodiments P18 to P20, wherein the enhancer is located upstream of the promoter sequence.

Embodiment P22: The rAAV according to any of embodiments P1 to P21, wherein the packaged genome further comprises one or more intron sequences.

Embodiment P23: The rAAV according to embodiment P22, wherein the intron is selected from an SV40 Small T intron, a rabbit hemoglobin subunit beta (rHBB) intron, a human beta globin IVS2 intron, a Promega chimeric intron, or an hFIX intron.

Embodiment P24: The rAAV according to embodiment P23, wherein the intron is the SV40 Small T intron.

Embodiment P25: The rAAV according to embodiment P24, wherein the intron comprises or consists of SEQ ID NO: 20.

Embodiment P26: The rAAV according to embodiment P23, wherein the intron is the rHBB intron.

Embodiment P27: The rAAV according to embodiment P26, wherein the intron comprises or consists of SEQ ID NO: 21.

Embodiment P28: The rAAV according to any of embodiments P1 to P27, wherein the packaged genome further comprises a polyadenylation signal sequence.

Embodiment P29: The rAAV according to embodiment P28, wherein the polyadenylation signal sequence is selected from a bovine growth hormone (BGH) polyadenylation signal sequence, an SV40 polyadenylation signal sequence, and a rabbit beta globin polyadenylation signal sequence.

Embodiment P30: The rAAV according to embodiment P29, wherein the polyadenylation signal sequence is the bovine growth hormone (BGH) polyadenylation signal sequence.

Embodiment P31: The rAAV according to embodiment P30, wherein the polyadenylation signal sequence comprises or consists of SEQ ID NO: 22.

Embodiment P32: The rAAV according to embodiment P29, wherein the polyadenylation signal sequence is the SV40 polyadenylation signal sequence.

Embodiment P33: The rAAV according to embodiment P32, wherein the polyadenylation signal sequence comprises or consists of SEQ ID NO: 23.

Embodiment P34: A recombinant adeno-associated virus (rAAV), said rAAV comprising an AAV capsid, and a vector genome packaged therein, said vector genome comprising in 5′ to 3′ order:
(a) a 5′ ITR sequence;
(b) a promoter sequence;
(c) a partial or complete coding sequence for PCCB; and
(d) a 3′ ITR sequence.

Embodiment P35: The rAAV according to embodiment P34, wherein the AAV capsid is from an AAV of serotype 1, 2, 3, 4, 5, 6, 7, 8, 9, 10, 11, 12, rh10, or hu37.

Embodiment P36: The rAAV according to embodiment P35, wherein the AAV capsid is from AAV9.

Embodiment P37: The rAAV according to embodiment P35, wherein the AAV capsid is from AAV8.

Embodiment P38: The rAAV according to embodiment P34, wherein the AAV capsid is an AAV9 variant capsid.

Embodiment P39: The rAAV according to any of embodiments P34 to P38, wherein the promoter is selected from a chicken β-actin (CBA) promoter, a cytomegalovirus immediate early gene (CMV) promoter, a transthyretin (TTR) promoter, a thyroxine binding globulin (TBG) promoter, an alpha-1 anti-trypsin (A1AT) promoter, and a CAG promoter.

Embodiment P40: The rAAV according to embodiment P39, wherein the promoter is the CBA promoter.

Embodiment P41: The rAAV according to any of embodiments P34 to P40, wherein the partial or complete coding sequence for PCCB is a wild-type coding sequence.

Embodiment P42: The rAAV according to embodiment P41, wherein the coding sequence for PCCB comprises SEQ ID NO: 7.

Embodiment P43: The rAAV according to any of embodiments P34 to P40, wherein the partial or complete coding sequence for PCCB is a codon-optimized coding sequence.

Embodiment P44: The rAAV according to embodiment P43, wherein the coding sequence for PCCB comprises a coding sequence selected from SEQ ID NOs: 8-12.

Embodiment P45: The rAAV according to any of embodiments P34 to P44, wherein the 5′ ITR sequence is from AAV2.

Embodiment P46: The rAAV according to any of embodiments P34 to P44, wherein the 3′ ITR sequence is from AAV2.

Embodiment P47: The rAAV according to any of embodiments P34 to P44, wherein the 5′ ITR sequence and the 3′ ITR sequence are from AAV2.

Embodiment P48: The rAAV according to any of embodiments P45 to P47, wherein the 5′ ITR sequence and the 3′ ITR sequence comprises or consists of SEQ ID NO: 15.

Embodiment P49: The rAAV according to any of embodiments P34 to P44, wherein the 5′ ITR sequence and/or the 3′ ITR sequence are from a non-AAV2 source.

Embodiment P50: The rAAV according to any of embodiments P34 to P49, wherein the packaged genome further comprises one or more enhancer sequences.

Embodiment P51: The rAAV according to embodiment P50, wherein the enhancer is selected from a cytomegalovirus immediate early gene (CMV) enhancer, a transthyretin enhancer (enTTR), a chicken β-actin (CBA) enhancer, an En34 enhancer, and an ApoE enhancer.

Embodiment P52: The rAAV according to embodiment P51, wherein the enhancer is the CMV enhancer.

Embodiment P53: The rAAV according to embodiment P52, wherein the enhancer comprises or consists of SEQ ID NO: 19.

Embodiment P54: The rAAV according to embodiments P51 to P53, wherein the enhancer is located upstream of the promoter sequence.

Embodiment P55: The rAAV according to any of embodiments P34 to P54, wherein the packaged genome further comprises one or more intron sequences.

Embodiment P56: The rAAV according to embodiment P55, wherein the intron is selected from an SV40 Small T intron, a rabbit hemoglobin subunit beta (rHBB) intron, a human beta globin IVS2 intron, a Promega chimeric intron, or an hFIX intron.

Embodiment P57: The rAAV according to embodiment P56, wherein the intron is the SV40 Small T intron.

Embodiment P58: The rAAV according to embodiment P57, wherein the intron comprises or consists of SEQ ID NO: 20.

Embodiment P59: The rAAV according to embodiment P56, wherein the intron is the rHBB intron.

Embodiment P60: The rAAV according to embodiment P59, wherein the intron comprises or consists of SEQ ID NO: 21.

Embodiment P61: The rAAV according to any of embodiments P34 to P60, wherein the packaged genome further comprises a polyadenylation signal sequence.

Embodiment P62: The rAAV according to embodiment P61, wherein the polyadenylation signal sequence is selected from a bovine growth hormone (BGH) polyadenylation signal sequence, an SV40 polyadenylation signal sequence, and a rabbit beta globin polyadenylation signal sequence.

Embodiment P63: The rAAV according to embodiment P62, wherein the polyadenylation signal sequence is the bovine growth hormone (BGH) polyadenylation signal sequence.

Embodiment P64: The rAAV according to embodiment P63, wherein the polyadenylation signal sequence comprises or consists of SEQ ID NO: 22.

Embodiment P65: The rAAV according to embodiment P62, wherein the polyadenylation signal sequence is the SV40 polyadenylation signal sequence.

Embodiment P66: The rAAV according to embodiment P65, wherein the polyadenylation signal sequence comprises or consists of SEQ ID NO: 23.

Embodiment P67: A recombinant adeno-associated virus (rAAV), said rAAV comprising an AAV capsid, and a vector genome packaged therein, said vector genome comprising as operably linked components in 5′ to 3′ order:
(a) a 5′ ITR sequence;
(b) an enhancer sequence;
(c) a promoter sequence;
(d) an intron sequence;
(e) a partial or complete coding sequence for PCCA;
(f) a polyadenylation signal sequence; and
(g) a 3′ ITR sequence.

Embodiment P68: A recombinant adeno-associated virus (rAAV), said rAAV comprising an AAV capsid, and a vector genome packaged therein, said vector genome comprising as operably linked components in 5′ to 3′ order:
(a) a 5′ ITR sequence;
(b) an enhancer sequence;
(c) a promoter sequence;
(d) an intron sequence;
(e) a partial or complete coding sequence for PCCB;
(f) a polyadenylation signal sequence; and
(g) a 3′ ITR sequence.

Embodiment P69: The rAAV according to embodiment P67 or embodiment P68, wherein the AAV capsid is from an AAV of serotype 1, 2, 3, 4, 5, 6, 7, 8, 9, 10, 11, 12, rh10, or hu37.

Embodiment P70: The rAAV according to embodiment P69, wherein the AAV capsid is from AAV9.

Embodiment P71: The rAAV according to embodiment P69, wherein the AAV capsid is from AAV8.

Embodiment P72: The rAAV according to embodiment P67 or embodiment P68, wherein the AAV capsid is an AAV9 variant capsid.

Embodiment P73: The rAAV according to any of embodiments P67 to P72, wherein the promoter is selected from a chicken β-actin (CBA) promoter, a cytomegalovirus immediate early gene (CMV) promoter, a transthyretin (TTR) promoter, a thyroxine binding globulin (TBG) promoter, an alpha-1 anti-trypsin (A1AT) promoter, and a CAG promoter.

Embodiment P74: The rAAV according to embodiment P73, wherein the promoter is the CBA promoter.

Embodiment P75: The rAAV according to embodiment P67, wherein the partial or complete coding sequence for PCCA is a wild-type coding sequence.

Embodiment P76: The rAAV according to embodiment P75, wherein the coding sequence for PCCA comprises SEQ ID NO: 1.

Embodiment P77: The rAAV according to embodiment P67, wherein the partial or complete coding sequence for PCCA is a codon-optimized coding sequence.

Embodiment P78: The rAAV according to embodiment P77, wherein the coding sequence for PCCA comprises a coding sequence selected from SEQ ID NOs: 2-6.

Embodiment P79: The rAAV according to embodiment P68, wherein the partial or complete coding sequence for PCCB is a wild-type coding sequence.

Embodiment P80: The rAAV according to embodiment P79, wherein the coding sequence for PCCB comprises SEQ ID NO: 7.

Embodiment P81: The rAAV according to embodiment P68, wherein the partial or complete coding sequence for PCCB is a codon-optimized coding sequence.

Embodiment P82: The rAAV according to embodiment P81, wherein the coding sequence for PCCB comprises a coding sequence selected from SEQ ID NOs: 8-12.

Embodiment P83: The rAAV according to any of embodiments P67 to P82, wherein the enhancer is selected from a cytomegalovirus immediate early gene (CMV) enhancer, a transthyretin enhancer (enTTR), a chicken β-actin (CBA) enhancer, an En34 enhancer, and an ApoE enhancer.

Embodiment P84: The rAAV according to embodiment P83, wherein the enhancer is the CMV enhancer.

Embodiment P85: The rAAV according to any of embodiments P67 to P84, wherein the intron is selected from an SV40 Small T intron, a rabbit hemoglobin subunit beta (rHBB) intron, a human beta globin IVS2 intron, a Promega chimeric intron, or an hFIX intron.

Embodiment P86: The rAAV according to embodiment P85, wherein the intron is the SV40 Small T intron.

Embodiment P87: The rAAV according to embodiment P85, wherein the intron is the rHBB intron.

Embodiment P88: The rAAV according to any of embodiments P67 to P87, wherein the polyadenylation signal sequence is selected from a bovine growth hormone (BGH) polyadenylation signal sequence, an SV40 polyadenylation signal sequence, and a rabbit beta globin polyadenylation signal sequence.

Embodiment P89: The rAAV according to embodiment P88, wherein the polyadenylation signal sequence is the bovine growth hormone (BGH) polyadenylation signal sequence.

Embodiment P90: The rAAV according to embodiment P88, wherein the polyadenylation signal sequence is the SV40 polyadenylation signal sequence.

Embodiment P91: A recombinant adeno-associated virus (rAAV) useful for the treatment of propionic acidemia (PA), said rAAV comprising an AAV capsid, and a vector genome packaged therein, said vector genome comprising as operably linked components in 5′ to 3′ order:
(a) a 5′ ITR sequence;
(b) an enhancer sequence;
(c) a promoter sequence;
(d) an intron sequence;
(e) a coding sequence for PCCA selected from SEQ ID NOs: 1-6;
(f) a polyadenylation signal sequence; and
(g) a 3′ ITR sequence.

Embodiment P92: A recombinant adeno-associated virus (rAAV) useful for the treatment of propionic acidemia (PA), said rAAV comprising an AAV capsid, and a vector genome packaged therein, said vector genome comprising as operably linked components in 5′ to 3′ order:
(a) a 5′ ITR sequence;
(b) an enhancer sequence;
(c) a promoter sequence;
(d) an intron sequence;
(e) a coding sequence for PCCB selected from SEQ ID NOs: 7-12;
(f) a polyadenylation signal sequence; and
(g) a 3′ ITR sequence.

Embodiment P93: The rAAV according to embodiment P91 or P92, wherein the AAV capsid is from AAV9.

Embodiment P94: A recombinant adeno-associated virus (rAAV) useful for the treatment of propionic acidemia (PA), said rAAV comprising an AAV9 capsid, and a vector genome packaged therein, said vector genome comprising as operably linked components in 5′ to 3′ order:
(a) a 5′ ITR sequence;
(b) an enhancer sequence;
(c) a promoter sequence;
(d) an intron sequence;
(e) a coding sequence for PCCA selected from SEQ ID NOs: 1-6;
(f) a polyadenylation signal sequence; and
(g) a 3′ ITR sequence.

Embodiment P95: A recombinant adeno-associated virus (rAAV) useful for the treatment of propionic acidemia (PA), said rAAV comprising an AAV9 capsid, and a vector genome packaged therein, said vector genome comprising as operably linked components in 5′ to 3′ order:
(a) a 5′ ITR sequence;
(b) an enhancer sequence;
(c) a promoter sequence;
(d) an intron sequence;
(e) a coding sequence for PCCB selected from SEQ ID NOs: 7-12;
(f) a polyadenylation signal sequence; and
(g) a 3′ ITR sequence.

Embodiment P96: A recombinant adeno-associated virus (rAAV) useful for the treatment of propionic acidemia (PA), said rAAV comprising an AAV9 capsid, and a vector genome packaged therein, said vector genome comprising as operably linked components in 5′ to 3′ order:
(a) an AAV2 5′ ITR sequence;
(b) a CMV enhancer sequence;
(c) a CBA promoter sequence;
(d) an rHBB or an SV40 Small T intron sequence;
(e) a coding sequence for PCCA selected from SEQ ID NOs: 1-6;
(f) a BGH or SV40 polyadenylation signal sequence; and
(g) an AAV2 3′ ITR sequence.

Embodiment P97: A recombinant adeno-associated virus (rAAV) useful for the treatment of propionic acidemia (PA), said rAAV comprising an AAV9 capsid, and a vector genome packaged therein, said vector genome comprising as operably linked components in 5′ to 3′ order:
(a) an AAV2 5′ ITR sequence;
(b) a CMV enhancer sequence;
(c) a CBA promoter sequence;
(d) an rHBB or an SV40 Small T intron sequence;
(e) a coding sequence for PCCB selected from SEQ ID NOs: 7-12;
(f) a BGH or SV40 polyadenylation signal sequence; and
(g) an AAV2 3′ ITR sequence.

Embodiment P98: The rAAV according to any of embodiments P67 to P97, wherein the vector genome comprises a consensus Kozak sequence located between vector genome elements (d) and (e).

Embodiment P99: The rAAV according to embodiment P98, wherein the consensus Kozak sequence comprises SEQ ID NO: 24.

Embodiment P100: A composition comprising the rAAV of any of the preceding embodiments and a pharmaceutically acceptable carrier.

Embodiment P101: A method of treating propionic acidemia (PA) in a human subject comprising administering to the human subject a therapeutically effective amount of an rAAV of any of embodiments P1 to P99 or a composition of embodiment P100.

Embodiment P102: A method of treating propionic acidemia (PA) in a human subject comprising administering to the human subject
(1) a therapeutically effective amount of composition comprising an rAAV of any of embodiments P1 to P33; and
(2) a therapeutically effective amount of composition comprising an rAAV of any of embodiments P34 to P66.

Embodiment P103: The method of embodiment P102, wherein the compositions of (1) and (2) are administered simultaneously.

Embodiment P104: The method of embodiment P102, wherein the compositions of (1) and (2) are administered sequentially.

Embodiment P105: The method of embodiment P102, wherein the compositions of (1) and (2) are administered separately.

Embodiment P106: A method of treating propionic acidemia (PA) in a human subject diagnosed with at least one mutation in PCCA, said method comprising administering to the human subject a therapeutically effective amount of composition comprising an rAAV of any of embodiments P1 to P33.

Embodiment P107: The method of embodiment P106, wherein said mutation in PCCA is selected from Table 1.

Embodiment P108: A method of treating propionic acidemia (PA) in a human subject diagnosed with at least one mutation in PCCB, said method comprising administering to the human subject a therapeutically effective amount of composition comprising an rAAV of any of embodiments P34 to P66.

Embodiment P109: The method of embodiment P108, wherein said mutation in PCCB is selected from Table 2.

Embodiment P110: The method of any of embodiments P101 to P109, wherein the recombinant virus, the rAAV, or the composition is administered subcutaneously, intramuscularly, intradermally, intraperitoneally, or intravenously.

Embodiment P111: The method of embodiment P110, wherein the rAAV or the composition is administered intravenously.

Embodiment P112: The method of any of embodiments P101 to P111, wherein the rAAV is administered at a dose of about 1×10¹¹ to about 1×10¹⁴ genome copies (GC)/kg.

Embodiment P113: The method of embodiment P112, wherein the rAAV is administered at a dose of about 1×10¹² to about 1×10¹³ genome copies (GC)/kg.

Embodiment P114: The method according to any of embodiments P110 to P113, wherein administering the rAAV comprises administration of a single dose of rAAV.

Embodiment P115: The method according to any of embodiments P110 to P113, wherein administering the rAAV comprises administration of multiple doses of rAAV.

Embodiment P116: A recombinant nucleic acid encoding a PCCA polypeptide of SEQ ID NO: 16, wherein the nucleic acid sequence is less than 80% identical to the wild-type coding sequence of SEQ ID NO: 1.

Embodiment P117: The recombinant nucleic acid of embodiment P116, wherein the nucleic acid sequence comprises a sequence which is at least 80% identical to a sequence selected from SEQ ID NOs: 2-6.

Embodiment P118: The recombinant nucleic acid of embodiment P117, wherein the nucleic acid sequence comprises a sequence selected from SEQ ID NOs: 2-6.

Embodiment P119: The recombinant nucleic acid of any of embodiments P116 to P118, wherein the nucleic acid sequence further comprises one or more stop codons selected from TGA, TAA, and TAG at the 3′ end.

Embodiment P120: A recombinant nucleic acid encoding a PCCB polypeptide of SEQ ID NO: 17, wherein the nucleic acid sequence is less than 80% identical to the wild-type coding sequence of SEQ ID NO: 7.

Embodiment P121: The recombinant nucleic acid of embodiment P120, wherein the nucleic acid sequence comprises a sequence which is at least 80% identical to a sequence selected from SEQ ID NOs: 8-12.

Embodiment P122: The recombinant nucleic acid of embodiment P121, wherein the nucleic acid sequence comprises a sequence selected from SEQ ID NOs: 8-12.

Embodiment P123: The recombinant nucleic acid of any of embodiments P120 to P122, wherein the nucleic acid sequence further comprises one or more stop codons selected from TGA, TAA, and TAG at the 3′ end.

Embodiment P124: A recombinant nucleic acid which comprises a sequence which is at least 80% identical to a sequence selected from SEQ ID NOs: 2-6.

Embodiment P125: The recombinant nucleic acid of embodiment P124, wherein the recombinant nucleic acid comprises a sequence which is at least 90% identical to a sequence selected from SEQ ID NOs: 2-6.

Embodiment P126: The recombinant nucleic acid of embodiment P125, wherein the recombinant nucleic acid comprises a sequence which is at least 95% identical to a sequence selected from SEQ ID NOs: 2-6.

Embodiment P127: A recombinant nucleic acid which comprises a sequence selected from SEQ ID NOs: 2-6.

Embodiment P128: The recombinant nucleic acid of any of embodiments P124 to P127, wherein the nucleic acid sequence further comprises one or more stop codons selected from TGA, TAA, and TAG at the 3′ end.

Embodiment P129: A recombinant nucleic acid which comprises a sequence which is at least 80% identical to a sequence selected from SEQ ID NOs: 7-12.

Embodiment P130: The recombinant nucleic acid of embodiment P129, wherein the recombinant nucleic acid comprises a sequence which is at least 90% identical to a sequence selected from SEQ ID NOs: 7-12.

Embodiment P131: The recombinant nucleic acid of embodiment P130, wherein the recombinant nucleic acid comprises a sequence which is at least 95% identical to a sequence selected from SEQ ID NOs: 2-6.

Embodiment P132: A recombinant nucleic acid which comprises a sequence selected from SEQ ID NOs: 7-12.

Embodiment P133: The recombinant nucleic acid of any of embodiments P129 to P132, wherein the nucleic acid sequence further comprises one or more stop codons selected from TGA, TAA, and TAG at the 3′ end.

Embodiment P134: An rAAV comprising a recombinant nucleic acid according to any of embodiments P116 to P133.

Embodiment P135: A host cell comprising a recombinant nucleic acid according to any of embodiments P116 to P133 or an rAAV of embodiment P134.

Embodiment P136: The host cell of embodiment P135, wherein said host cell is selected from a HeLa, Cos-7, HEK293, A549, BHK, Vero, RD, HT-1080, ARPE-19, or MRC-5 cell.

### INCORPORATION BY REFERENCE

The entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Various structural elements of the different embodiments and various disclosed method steps may be utilized in various combinations and permutations, and all such variants are to be considered forms of the disclosure. Scope of the disclosure is thus indicated by the appended claims rather than by the foregoing description, and all changes that come within the meaning and range of equivalency of the claims are intended to be embraced therein.

What is claimed is:

1. A recombinant adeno-associated virus (rAAV), said rAAV comprising an AAV capsid, and a vector genome packaged therein, said vector genome comprising in 5′ to 3′ order:
(a) a 5′-inverted terminal repeat sequence (5′-ITR) sequence;
(b) a promoter sequence;
(c) a partial or complete coding sequence for propionyl-CoA carboxylase A (PCCA); and
(d) a 3′-inverted terminal repeat sequence (3′-ITR) sequence.

2. The rAAV according to claim 1, wherein the AAV capsid is from an AAV of serotype 9 or variant thereof, 1, 2, 3, 4, 5, 6, 7, 8, 10, 11, 12, rh10, or hu37.

3. The rAAV according to claim 1, wherein the promoter sequence is selected from a chicken β-actin (CBA) promoter sequence, a cytomegalovirus (CMV) immediate early gene promoter sequence, a transthyretin (TTR) promoter sequence, a thyroxine binding globulin (TBG) promoter sequence, an alpha-1 anti-trypsin (A1AT) promoter sequence, a CAG promoter sequence, and a PCCA gene-specific endogenous promoter sequence.

4. The rAAV according to claim 3, wherein:
(a) the promoter sequence is:
i. the PCCA gene-specific endogenous promoter sequence comprising a nucleotide sequence of at least 15 continuous nucleotides, which is at least 95% identical to an equal length region of SEQ ID NO: 34; or
ii. the CBA promoter sequence; and/or
(b) the 5′-ITR and/or the 3′-ITR sequence:
i. are from AAV2;
ii. comprises or consists of SEQ ID NO: 15; or
iii. are from a non-AAV2 source.

5. The rAAV according to claim 1, wherein the partial or complete coding sequence for PCCA is:
i. a wild-type coding sequence comprising SEQ ID NO: 1; or
ii. a codon-optimized coding sequence comprising a coding sequence selected from SEQ ID NOs: 2-6.

6. The rAAV according to claim 1, wherein the vector genome further comprises—a truncated or complete nucleotide sequence of a human PCCA 5′-untranslated region (UTR) and/or a 3′-UTR.

7. The rAAV according to claim 6, wherein:
(a) the complete nucleotide sequence of the human PCCA 5′-UTR comprises SEQ ID NO: 30;
(b) the truncated nucleotide sequence of the human PCCA 5′-UTR comprises a nucleotide sequence of at least 100 continuous nucleotides, which is at least 95% identical to an equal length region of SEQ ID NO: 30;
(c) the complete nucleotide sequence of the human PCCA 3′-UTR comprises SEQ ID NO: 31; or
(d) the truncated nucleotide sequence of the human PCCA 3′-UTR comprises a nucleotide sequence of at least 15 continuous nucleotides, which is at least 95% identical to an equal length region of SEQ ID NO: 31.

8. The rAAV according to claim 1, wherein the vector genome further comprises:
(a) a polyadenylation signal sequence;
(b) one or more enhancer sequences; and/or
(c) one or more intron sequences.

9. The rAAV of claim 1, wherein the vector genome comprises in 5′ to 3′ order:
(a) a 5′-ITR sequence;
(b) an enhancer sequence;
(c) a promoter sequence;
(d) an intron sequence;
(e) a wild type or a codon-optimized coding sequence for PCCA;
(f) a polyadenylation signal sequence; and
(g) a 3′-ITR sequence.

10. The rAAV according to claim 9, wherein:
(a) the polyadenylation signal sequence:
i. is selected from an SV40 polyadenylation signal sequence, a bovine growth hormone (BGH) polyadenylation signal sequence, a rabbit beta globin polyadenylation signal sequence, and a PCCA gene-specific endogenous polyadenylation signal sequence;
ii. is a bovine growth hormone (BGH) polyadenylation signal sequence comprising SEQ ID NO: 22;
iii. is an SV40 polyadenylation signal sequence comprising SEQ ID NO: 23; or
iv. comprises the PCCA gene-specific endogenous polyadenylation signal sequence; or
v. is a PCCA gene-specific endogenous polyadenylation signal sequence comprising a nucleotide sequence of at least 15 continuous nucleotides, which is at least 95% identical to an equal length region of SEQ ID NO: 35;
(b) the enhancer sequence:
i. is selected from a cytomegalovirus (CMV) immediate early gene enhancer sequence, a transthyretin enhancer (enTTR) sequence, a chicken β-actin (CBA) enhancer sequence, an En34 enhancer sequence, and an apolipoprotein E (ApoE) enhancer sequence;
ii. comprises or consists of SEQ ID NO: 19; and/or
iii. is located upstream of the promoter sequence; and/or
(c) the intron sequence is:
i. selected from a human beta globin IVS2 intron sequence, an SV40 Small T intron sequence, a rabbit hemoglobin subunit beta (rHBB) intron sequence, a Promega chimeric intron sequence, and an hFIX intron sequence; or
ii. an SV40 Small T intron sequence comprising or consisting of SEQ ID NO: 20 or a rHBB intron sequence comprising or consisting of SEQ ID NO: 21.

11. The rAAV of claim 1, wherein the vector genome comprises in 5′ to 3′ order:
(a) the 5′-inverted terminal repeat (ITR) sequence;
(b) the promoter sequence;
(c) a human beta globin IVS2 intron sequence;
(d) the wild type coding sequence for PCCA according to SEQ ID NO: 1, or the codon optimized coding sequence for PCCA selected from SEQ ID NOs: 2-6; and
(e) the 3′-inverted terminal repeat sequence (ITR) sequence.

12. The rAAV of claim 1, wherein the rAAV comprises an AAV9 capsid and the vector genome comprises in 5′ to 3′ order:
(a) the 5′-inverted terminal repeat sequence (ITR) sequence;
(b) an enhancer sequence;
(c) the promoter sequence;
(d) an intron sequence;
(e) the wild type coding sequence for PCCA according to SEQ ID NO: 1, or the codon optimized coding sequence for PCCA selected from SEQ ID NOs: 2-6;
(f) a polyadenylation signal sequence; and
(g) the 3′-inverted terminal repeat sequence (ITR) sequence.

13. The rAAV of claim 1, wherein the rAAV comprises an AAV9 capsid and the vector genome comprises in 5′ to 3′ order:
(a) an AAV2 5′-inverted terminal repeat sequence (ITR) sequence;
(b) a CMV enhancer sequence;
(c) a CBA promoter sequence;
(d) a human beta globin IVS2 intron, an rHBB or an SV40 Small T intron sequence;
(e) the wild type coding sequence for PCCA according to SEQ ID NO: 1, or the codon optimized coding sequence for PCCA selected from SEQ ID NOs: 2-6;
(f) a BGH or SV40 polyadenylation signal sequence; and
(g) an AAV2 3′-inverted terminal repeat sequence (ITR) sequence.

14. The rAAV according to claim 13, wherein the vector genome further comprises:
(a) a truncated or complete nucleotide sequence of a human PCCA 5′-UTR located between vector genome elements (d) and (e);
(b) a truncated or complete nucleotide sequence of a human PCCA 3′-UTR located between vector genome elements (f) and (g); and/or
(c) a consensus Kozak sequence comprising SEQ ID NO: 24 upstream of genome element (e).

15. A composition comprising the rAAV of claim 1, and a pharmaceutically acceptable carrier.

16. A method of treating propionic acidemia (PA) in a human subject comprising administering to the human subject a therapeutically effective amount of the rAAV of claim 1.

17. A method of treating propionic acid (PA) in a human subject comprising administering to the human subject a therapeutically effective amount of a recombinant adeno-associated virus (rAAV) comprising an AAV capsid, and a vector genome packaged therein, said vector genome comprising in 5′ to 3′ order:
(a) a 5′-inverted terminal repeat (5′-ITR) sequence;
(b) a promoter sequence;
(c) a human beta globin IVS2 intron sequence;
(d) a wild type coding sequence for propionyl-CoA carboxylase B (PCCB) according to SEQ ID NO: 7; and
(e) a 3′-inverted terminal repeat sequence (3′-ITR) sequence.

18. A recombinant nucleic acid comprising:
a) a vector genome sequence comprising a coding sequence for a propionyl-CoA carboxylase A (PCCA) polypeptide of SEQ ID NO: 16, wherein the coding sequence for PCCA:
i. is less than 80% identical to the wild-type coding sequence of SEQ ID NO: 1;
ii. is at least 80% identical to a codon optimized sequence selected from SEQ ID NOs: 2-6; or
iii. comprises a codon optimized sequence selected from SEQ ID NOs: 2-6; or
b) a vector genome sequence comprising a coding sequence for a propionyl-CoA carboxylase B (PCCB) polypeptide sequence of SEQ ID NO: 17, wherein the coding sequence for PCCB:
i. is less than 80% identical to the wild-type coding sequence of SEQ ID NO: 7;
ii. is at least 80% identical to a codon optimized sequence selected from SEQ ID NOs: 8-12; or
iii. comprises a codon optimized sequence selected from SEQ ID NOs: 8-12;

19. An rAAV comprising the recombinant nucleic acid according to claim 18.

20. A host cell comprising the recombinant nucleic acid of claim 18, wherein said host cell is selected from a HeLa, Cos-7, HEK293, A549, BHK, Vero, RD, HT-1080, ARPE-19, and MRC-5 cell.


SEQUENCE LISTING (39 sequences)

SEQ ID NO: 1 - DNA, 2184 bp, Homo sapiens
  source 1..2184: mol_type = other DNA
atggcggggttctgggtcgggacagcaccgctggtcgctgccggacggcgtgggcggtggccgccgcagcagctgatgctgagcgcggcgctgcggaccctgaagcatgttctgtactattcaagacagtgcttaatggtgtcccgtaatcttggttcagtgggatatgatcctaatgaaaaaacttttgataaaattcttgttgctaatagaggagaaattgcatgtcgggttattagaacttgcaagaagatgggcattaagacagttgccatccacagtgatgttgatgctagttctgttcatgtgaaaatggcggatgaggctgtctgtgttggcccagctcccaccagtaaaagctacctcaacatggatgccatcatggaagccattaagaaaaccagggcccaagctgtacatccaggttatggattcctttcagaaaacaaagaatttgccagatgtttggcagcagaagatgtcgttttcattggacctgacacacatgctattcaagccatgggcgacaagattgaaagcaaattattagctaagaaagcagaggttaatacaatccctggctttgatggagtagtcaaggatgcagaagaagctgtcagaattgcaagggaaattggctaccctgtcatgatcaaggcctcagcaggtggtggtgggaaaggcatgcgcattgcttgggatgatgaagagaccagggatggttttagattgtcatctcaagaagctgcttctagttttggcgatgatagactactaatagaaaaatttattgataatcctcgtcatatagaaatccaggttctaggtgataaacatgggaatgctttatggcttaatgaaagagagtgctcaattcagagaagaaatcagaaggtggtggaggaagcaccaagcatttttttggatgcggagactcgaagagcgatgggagaacaagctgtagctcttgccagagcagtaaaatattcctctgctgggaccgtggagttccttgtggactctaagaagaatttttatttcttggaaatgaatacaagactccaggttgagcatcctgtcacagaatgcattactggcctggacctagtccaggaaatgatccgtgttgctaagggctaccctctcaggcacaaacaagctgatattcgcatcaacggctgggcagttgaatgtcgggtttatgctgaggacccctacaagtcttttggtttaccatctattgggagattgtctcagtaccaagaaccgttacatctacctggtgtccgagtggacagtggcatccaaccaggaagtgatattagcatttattatgatcctatgatttcaaaactaatcacatatggctctgatagaactgaggcactgaagagaatggcagatgcactggataactatgttattcgaggtgttacacataatattgcattacttcgagaggtgataatcaactcacgctttgtaaaaggagacatcagcactaaatttctctccgatgtgtatcctgatggcttcaaaggacacatgctaaccaagagtgagaagaaccagttattggcaatagcatcatcattgtttgtggcattccagttaagagcacaacattttcaagaaaattcaagaatgcctgttattaaaccagacatagccaactgggagctctcagtaaaattgcatgataaagttcataccgtagtagcatcaaacaatgggtcagtgttctcggtggaagttgatgggtcgaaactaaatgtgaccagcacgtggaacctggcttcgcccttattgtctgtcagcgttgatggcactcagaggactgtccagtgtctttctcgagaagcaggtggaaacatgagcattcagtttcttggtacagtgtacaaggtgaatatcttaaccagacttgccgcagaattgaacaaatttatgctggaaaaagtgactgaggacacaagcagtgttctgcgttccccgatgcccggagtggtggtggccgtctctgtcaagcctggagacgcggtagcagaaggtcaagaaatttgtgtgattgaagccatgaaaatgcagaatagtatgacagctgggaaaactggcacggtgaaatctgtgcactgtcaagctggagacacagttggagaaggggatctgctcgtggagctggaa

SEQ ID NO: 2 - DNA, 2184 bp, synthetic construct
  misc_feature 1..2184: note = Description of Artificial Sequence: PCCA Coding Sequence - Codon-Optimized
  source 1..2184: mol_type = other DNA
atggctggattttgggtcggaacggcccctctcgtggccgccggccgccggggtcggtggccgccgcaacagctgatgttgtcggccgcgctgcgcacccttaagcatgtgctgtactactcccggcaatgccttatggtgtccagaaacctgggtagcgtgggctatgacccgaacgagaaaaccttcgacaagattctggtggccaaccggggggaaattgcctgccgggtcatcaggacttgcaagaagatgggcatcaagaccgtcgccattcactccgacgtggacgcctcctccgtgcacgtgaagatggcagatgaagccgtctgcgtgggccccgccccgacctccaagtcctaccttaacatggacgcgatcatggaagccatcaaaaagaccagagcccaggcagtgcacccgggatacggctttctctccgaaaacaaggagttcgcgcggtgcctggccgctgaagatgtcgtgttcatcggccctgatacccacgcgatccaggctatgggagacaagatcgaatccaagctgctcgccaagaaagccgaagtcaacaccatacctgggtttgacggcgtggtcaaggacgcagaagaagccgtcaggattgcccgcgagatcggataccccgtgatgatcaaggcatccgccggggggggaggaaagggaatgcgcatcgcctgggatgacgaagaaacccgggacggcttcagactctcgtcacaagaggccgcgtcctcattcggggatgaccggctcctgattgagaagttcattgacaatcctcggcacatcgagattcaggtcctgggcgataagcatggaaacgccctgtggctgaacgaacgcgaatgcagcatccagaggcggaaccagaaagtggtggaagaggccccatccatctttctcgacgccgagactcggagagcgatgggtgaacaggccgtggccctggcccgagccgtgaagtactccagcgcggggactgtcgagttcctggtggacagcaagaagaatttctacttcctggagatgaatactcggctccaagtggaacaccccgtgaccgaatgcattaccggtctggacctcgtccaagaaatgatccgcgtcgccaagggctacccattgagacacaaacaggccgacattcggatcaacggatgggccgtcgagtgtcgcgtgtacgcggaagatccgtataagtcgttcggactgccgtccattggtagactctcgcagtaccaagagccactgcacctccccggagtgcgcgtggactcaggcatccagcccggaagcgacatctctatctactacgaccccatgatttccaagttgatcacctacgggtccgataggaccgaggcactgaagcgcatggctgacgcacttgacaactacgtgatccgcggggtcactcacaacattgccctgctccgcgaagtgatcatcaactcgcgcttcgtgaagggcgacatctccactaagttcctgtccgacgtgtaccctgacggtttcaagggccatatgctgaccaagtccgagaagaaccagctcctggctatcgcctcctccctgtttgtggcgttccagctgagggcgcagcacttccaggagaacagccggatgcccgtgatcaagcctgacatcgccaattgggagctgtccgtgaagctgcacgataaggtccataccgtggtggcatccaacaacggatcggtgttcagcgtggaagtggacgggtccaagctgaacgtgaccagcacatggaacctggcgtcccccctgttgtctgtgtcggtcgatggcacgcagcgcactgtgcagtgcctctcccgggaagctggcggaaacatgagcatccagttcctgggtactgtgtacaaggtcaacattctgactcggctggccgccgagctgaacaagttcatgttggaaaaagtcaccgaagatacatcgtcagtcctgcggagcccaatgcctggagtcgtggtggcggtgtcagtgaagcccggcgatgctgtggccgaaggccaagagatctgcgtgatcgaggccatgaagatgcagaactcgatgaccgccggaaagaccggtaccgtgaagtccgtgcattgtcaagcgggcgacactgtgggagagggagatctgctcgtggagctggag

SEQ ID NO: 3 - DNA, 2184 bp, synthetic construct
  misc_feature 1..2184: note = Description of Artificial Sequence: PCCA Coding Sequence - Codon-Optimized
  source 1..2184: mol_type = other DNA
atggccggcttctgggtcggcaccgcccctctggtcgcggccggacgacgcggacgctggccaccccagcaactgatgctgagcgcggccttgaggactctgaagcacgtgctctactactcgcggcagtgcctgatggtgtcccggaatctggggtccgtgggatacgaccctaacgaaaagaccttcgataagatcctcgtggcaaatcggggagagatcgcgtgtcgcgtgatccgcacgtgcaagaagatggggatcaagactgtggcaatccatagcgatgtggatgcatcctcggtccacgtgaagatggccgacgaagctgtgtgcgtgggaccggcgccgacttcgaaatcgtacctgaacatggacgctattatggaggcgatcaagaaaacgcgcgcccaagcggtccatcccggttacggattcctgagcgagaacaaggaatttgcacggtgcctcgctgccgaggacgtggtgtttatcggtcccgacacccacgccatccaagctatgggggacaagattgagtccaagctcctggcgaaaaaggcagaggtcaacacaattcctggtttcgacggcgtcgtgaaggacgccgaagaagccgtgcgcatcgcgagggaaatcggttaccctgtgatgattaaggcctccgccggcggcggtggaaagggaatgagaattgcctgggacgatgaagaaacccgcgacggattccgcctgtcgagccaggaagccgcctcttccttcggcgatgacagactgctgatcgaaaagttcatcgataaccccagacacattgagatccaagtgctcggggataagcacggcaacgccctttggctgaacgagagagagtgctccattcaacgccgcaatcagaaggtcgtggaggaagccccgtcgatattcctggatgccgaaacccggcgggccatgggagagcaggctgtcgcgttggcgcgggccgtcaagtacagctcggccgggaccgtggaatttctggtcgattccaagaagaacttctatttcctggagatgaacaccagactccaggtcgagcacccggtcactgagtgtatcaccgggctcgatctggtgcaagagatgattcgggtggcgaagggatatccccttcggcataaacaagccgacatcaggatcaacggttgggccgtggaatgcagggtctacgccgaggacccctacaagagcttcggcctgcccagcatcggccgcctgtcacagtatcaggaaccgctgcatcttccgggcgtgcgggtcgacagcggaattcagcctggctcagatatctccatctactacgatccaatgatctcaaagctgattacttatggatccgaccggaccgaagcccttaagcgaatggccgacgccctggacaactacgtgatccggggagtgacccacaacatcgccttgctgcgggaagtgatcattaacagcagattcgtgaagggagacatcagcaccaagttcctgtcggatgtctacccggacgggttcaaagggcacatgcttactaagtccgagaagaatcagctgctcgccattgcgtcaagcttgttcgtggcctttcaactccgggcccagcacttccaggaaaactcccgcatgccagtcattaagccggacatcgccaactgggaactcagcgtgaagctccatgacaaagtgcataccgtggtggccagcaacaacggtagcgtgttctcagtcgaggtcgatggctcgaagctcaacgtcacttccacttggaacttggccagcccgctgctgtccgtgtccgtggacggaacccagaggaccgtgcagtgtctgtcgagagaagccggcggcaacatgtcaatccagttcctgggaaccgtgtacaaggtcaacatcctgaccagactggccgccgaactgaacaagtttatgctcgagaaagtgaccgaggacactagctccgtgctgcgctcccctatgcccggagtggtcgtggcagtgtccgtgaagccgggcgacgccgtggccgagggacaggaaatctgtgtgatcgaagcgatgaagatgcagaattcaatgaccgcgggaaagactgggaccgtgaagtctgtgcactgccaggctggcgataccgtgggggagggcgaccttctggtggaactcgag

SEQ ID NO: 4 - DNA, 2184 bp, synthetic construct
  misc_feature 1..2184: note = Description of Artificial Sequence: PCCA Coding Sequence - Codon-Optimized
  source 1..2184: mol_type = other DNA
atggctgggttttgggtggggacagctcctctggtggctgctgggaggagggggaggtggcctcctcagcagctgatgctgtctgctgctctgaggacactgaagcatgtgctgtattattctaggcagtgtctgatggtgtctaggaatctggggtctgtggggtatgatcctaatgagaagacatttgataagattctggtggctaataggggggagattgcttgtagggtgattaggacatgtaagaagatggggattaagacagtggctattcattctgatgtggatgcttcttctgtgcatgtgaagatggctgatgaggctgtgtgtgtggggcctgctcctacatctaagtcttatctgaatatggatgctattatggaggctattaagaagacaagggctcaggctgtgcatcctgggtatgggtttctgtctgagaataaggagtttgctaggtgtctggctgctgaggatgtggtgtttattgggcctgatacacatgctattcaggctatgggggataagattgagtctaagctgctggctaagaaggctgaggtgaatacaattcctgggtttgatggggtggtgaaggatgctgaggaggctgtgaggattgctagggagattgggtatcctgtgatgattaaggcttctgctggggggggggggaaggggatgaggattgcttgggatgatgaggagacaagggatgggtttaggctgtcttctcaggaggctgcttcttcttttggggatgataggctgctgattgagaagtttattgataatcctaggcatattgagattcaggtgctgggggataagcatgggaatgctctgtggctgaatgagagggagtgttctattcagaggaggaatcagaaggtggtggaggaggctccttctatttttctggatgctgagacaaggagggctatgggggagcaggctgtggctctggctagggctgtgaagtattcttctgctgggacagtggagtttctggtggattctaagaagaatttttattttctggagatgaatacaaggctgcaggtggagcatcctgtgacagagtgtattacagggctggatctggtgcaggagatgattagggtggctaaggggtatcctctgaggcataagcaggctgatattaggattaatgggtgggctgtggagtgtagggtgtatgctgaggatccttataagtcttttgggctgccttctattgggaggctgtctcagtatcaggagcctctgcatctgcctggggtgagggtggattctgggattcagcctgggtctgatatttctatttattatgatcctatgatttctaagctgattacatatgggtctgataggacagaggctctgaagaggatggctgatgctctggataattatgtgattaggggggtgacacataatattgctctgctgagggaggtgattattaattctaggtttgtgaagggggatatttctacaaagtttctgtctgatgtgtatcctgatgggtttaaggggcatatgctgacaaagtctgagaagaatcagctgctggctattgcttcttctctgtttgtggcttttcagctgagggctcagcattttcaggagaattctaggatgcctgtgattaagcctgatattgctaattgggagctgtctgtgaagctgcatgataaggtgcatacagtggtggcttctaataatgggtctgtgttttctgtggaggtggatgggtctaagctgaatgtgacatctacatggaatctggcttctcctctgctgtctgtgtctgtggatgggacacagaggacagtgcagtgtctgtctagggaggctggggggaatatgtctattcagtttctggggacagtgtataaggtgaatattctgacaaggctggctgctgagctgaataagtttatgctggagaaggtgacagaggatacatcttctgtgctgaggtctcctatgcctggggtggtggtggctgtgtctgtgaagcctggggatgctgtggctgaggggcaggagatttgtgtgattgaggctatgaagatgcagaattctatgacagctgggaagacagggacagtgaagtctgtgcattgtcaggctggggatacagtgggggagggggatctgctggtggagctggag

SEQ ID NO: 5 - DNA, 2184 bp, synthetic construct
  misc_feature 1..2184: note = Description of Artificial Sequence: PCCA Coding Sequence - Codon-Optimized
  source 1..2184: mol_type = other DNA
atggcagggttttgggtggggacagcaccactggtggcagcagggaggagggggaggtggccaccacagcagctgatgctgtcagcagcactgaggacactgaagcatgtgctgtattattcaaggcagtgtctgatggtgtcaaggaatctggggtcagtggggtatgatccaaatgagaagacatttgataagattctggtggcaaataggggggagattgcatgtagggtgattaggacatgtaagaagatggggattaagacagtggcaattcattcagatgtggatgcatcatcagtgcatgtgaagatggcagatgaggcagtgtgtgtggggccagcaccaacatcaaagtcatatctgaatatggatgcaattatggaggcaattaagaagacaagggcacaggcagtgcatccagggtatgggtttctgtcagagaataaggagtttgcaaggtgtctggcagcagaggatgtggtgtttattgggccagatacacatgcaattcaggcaatgggggataagattgagtcaaagctgctggcaaagaaggcagaggtgaatacaattccagggtttgatggggtggtgaaggatgcagaggaggcagtgaggattgcaagggagattgggtatccagtgatgattaaggcatcagcaggggggggggggaaggggatgaggattgcatgggatgatgaggagacaagggatgggtttaggctgtcatcacaggaggcagcatcatcatttggggatgataggctgctgattgagaagtttattgataatccaaggcatattgagattcaggtgctgggggataagcatgggaatgcactgtggctgaatgagagggagtgttcaattcagaggaggaatcagaaggtggtggaggaggcaccatcaatttttctggatgcagagacaaggagggcaatgggggagcaggcagtggcactggcaagggcagtgaagtattcatcagcagggacagtggagtttctggtggattcaaagaagaatttttattttctggagatgaatacaaggctgcaggtggagcatccagtgacagagtgtattacagggctggatctggtgcaggagatgattagggtggcaaaggggtatccactgaggcataagcaggcagatattaggattaatgggtgggcagtggagtgtagggtgtatgcagaggatccatataagtcatttgggctgccatcaattgggaggctgtcacagtatcaggagccactgcatctgccaggggtgagggtggattcagggattcagccagggtcagatatttcaatttattatgatccaatgatttcaaagctgattacatatgggtcagataggacagaggcactgaagaggatggcagatgcactggataattatgtgattaggggggtgacacataatattgcactgctgagggaggtgattattaattcaaggtttgtgaagggggatatttcaacaaagtttctgtcagatgtgtatccagatgggtttaaggggcatatgctgacaaagtcagagaagaatcagctgctggcaattgcatcatcactgtttgtggcatttcagctgagggcacagcattttcaggagaattcaaggatgccagtgattaagccagatattgcaaattgggagctgtcagtgaagctgcatgataaggtgcatacagtggtggcatcaaataatgggtcagtgttttcagtggaggtggatgggtcaaagctgaatgtgacatcaacatggaatctggcatcaccactgctgtcagtgtcagtggatgggacacagaggacagtgcagtgtctgtcaagggaggcaggggggaatatgtcaattcagtttctggggacagtgtataaggtgaatattctgacaaggctggcagcagagctgaataagtttatgctggagaaggtgacagaggatacatcatcagtgctgaggtcaccaatgccaggggtggtggtggcagtgtcagtgaagccaggggatgcagtggcagaggggcaggagatttgtgtgattgaggcaatgaagatgcagaattcaatgacagcagggaagacagggacagtgaagtcagtgcattgtcaggcaggggatacagtgggggagggggatctgctggtggagctggag

SEQ ID NO: 6 - DNA, 2184 bp, synthetic construct
  misc_feature 1..2184: note = Description of Artificial Sequence: PCCA Coding Sequence - Codon-Optimized
  source 1..2184: mol_type = other DNA
atggctggtttttgggtaggtacagcaccactagtagcagcaggtaggaggggtaggtggccaccacaacaactaatgctatcagcagcactaaggacactaaagcatgtactatattattcaaggcaatgtctaatggtatcaaggaatctggggtctgtggggtatgatcctaatgagaagacatttgataagattctggtggctaataggggggagattgcttgtagggtgattaggacatgtaagaagatggggattaagacagtggctattcattctgatgtggatgcttcttctgtgcatgtgaagatggctgatgaggctgtgtgtgtggggcctgctcctacatctaagtcttatctgaatatggatgctattatggaggctattaagaagacaagggctcaggctgtgcatcctgggtatgggtttctgtctgagaataaggagtttgctaggtgtctggctgctgaggatgtggtgtttattgggcctgatacacatgctattcaggctatgggggataagattgagtctaagctgctggctaagaaggctgaggtgaatacaattcctgggtttgatggggtggtgaaggatgctgaggaggctgtgaggattgctagggagattgggtatcctgtgatgattaaggcttctgctggggggggggggaaggggatgaggattgcttgggatgatgaggagacaagggatgggtttaggctgtcttctcaggaggctgcttcttcttttggggatgataggctgctgattgagaagtttattgataatcctaggcatattgagattcaggtgctgggggataagcatgggaatgctctgtggctgaatgagagggagtgttctattcagaggaggaatcagaaggtggtggaggaggctccttctatttttctggatgctgagacaaggagggctatgggggagcaggctgtggctctggctagggctgtgaagtattcttctgctgggacagtggagtttctggtggattctaagaagaatttttattttctggagatgaatacaaggctgcaggtggagcatcctgtgacagagtgtattacagggctggatctggtgcaggagatgattagggtggctaaggggtatcctctgaggcataagcaggctgatattaggattaatgggtgggctgtggagtgtagggtgtatgctgaggatccttataagtcttttgggctgccttctattgggaggctgtctcagtatcaggagcctctgcatctgcctggggtgagggtggattctgggattcagcctgggtctgatatttctatttattatgatcctatgatttctaagctgattacatatgggtctgataggacagaggctctgaagaggatggctgatgctctggataattatgtgattaggggggtgacacataatattgctctgctgagggaggtgattattaattctaggtttgtgaagggggatatttctacaaagtttctgtctgatgtgtatcctgatgggtttaaggggcatatgctgacaaagtctgagaagaatcagctgctggctattgcttcttctctgtttgtggcttttcagctgagggctcagcattttcaggagaattctaggatgcctgtgattaagcctgatattgctaattgggagctgtctgtgaagctgcatgataaggtgcatacagtggtggcttctaataatgggtctgtgttttctgtggaggtggatgggtctaagctgaatgtgacatctacatggaatctggcttctcctctgctgtctgtgtctgtggatgggacacagaggacagtgcagtgtctgtctagggaggctggggggaatatgtctattcagtttctggggacagtgtataaggtgaatattctgacaaggctggctgctgagctgaataagtttatgctggagaaggtgacagaggatacatcttctgtgctgaggtctcctatgcctggggtggtggtggctgtgtctgtgaagcctggggatgctgtggctgaggggcaggagatttgtgtgattgaggctatgaagatgcagaattctatgacagctgggaagacagggacagtgaagtctgtgcattgtcaggctggggatacagtgggggagggggatctgctggtggagctggag

SEQ ID NO: 7 - DNA, 1617 bp, Homo sapiens
  source 1..1617: mol_type = other DNA
atggcggcggcattacgggtggcggcggtcggggcaaggctcagcgttctggcgagcggtctccgcgccgcggtccgcagcctttgcagccaggccacctctgttaacgaacgcatcgaaaacaagcgccggaccgcgctgctgggagggggccaacgccgtattgacgcgcagcacaagcgaggaaagctaacagccagggagaggatcagtctcttgctggaccctggcagctttgttgagagcgacatgtttgtggaacacagatgtgcagattttggaatggctgctgataagaataagtttcctggagacagcgtggtcactggacgaggccgaatcaatggaagattggtttatgtcttcagtcaggattttacagtttttggaggcagtctgtcaggagcacatgcccaaaagatctgcaaaatcatggaccaggccataacggtgggggctccagtgattgggctgaatgactctgggggagcacggatccaagaaggagtggagtctttggctggctatgcagacatctttctgaggaatgttacggcatccggagtcatccctcagatttctctgatcatgggcccatgtgctggtggggccgtctactccccagccctaacagacttcacgttcatggtaaaggacacctcctacctgttcatcactggccctgatgttgtgaagtctgtcaccaatgaggatgttacccaggaggagctcggtggtgccaagacccacaccaccatgtcaggtgtggcccacagagcttttgaaaatgatgttgatgccttgtgtaatctccgggatttcttcaactacctgcccctgagcagtcaggacccggctcccgtccgtgagtgccacgatcccagtgaccgtctggttcctgagcttgacacaattgtccctttggaatcaaccaaagcctacaacatggtggacatcatacactctgttgttgatgagcgtgaattttttgagatcatgcccaattatgccaagaacatcattgttggttttgcaagaatgaatgggaggactgttggaattgttggcaaccaacctaaggtggcctcaggatgcttggatattaattcatctgtgaaaggggctcgttttgtcagattctgtgatgcattcaatattccactcatcacttttgttgatgtccctggctttctacctggcacagcacaggaatacgggggcatcatccggcatggtgccaagcttctctacgcatttgctgaggcaactgtacccaaagtcacagtcatcaccaggaaggcctatggaggtgcctatgatgtcatgagctctaagcacctttgtggtgataccaactatgcctggcccaccgcagagattgcagtcatgggagcaaagggcgctgtggagatcatcttcaaagggcatgagaatgtggaagctgctcaggcagagtacatcgagaagtttgccaaccctttccctgcagcagtgcgagggtttgtggatgacatcatccaaccttcttccacacgtgcccgaatctgctgtgacctggatgtcttggccagcaagaaggtacaacgtccttggagaaaacatgcaaatattccattg

SEQ ID NO: 8 - DNA, 1617 bp, synthetic construct
  misc_feature 1..1617: note = Description of Artificial Sequence: PCCB Coding Sequence - Codon-Optimized
  source 1..1617: mol_type = other DNA
atggctgccgccctgcgcgtggcggccgtgggagcaagactgtccgtgctggcgtcgggcttgagagcggccgtgcggagcctgtgctcacaagcaacctcggtgaacgaacgcatcgagaacaagcgcaggactgcgctgctgggcgggggccagcgcaggatcgacgcacagcataagcgcggaaagctgaccgcccgcgagcggatttccctgctcctggatcctggaagcttcgtggagtccgacatgttcgtggagcaccgctgcgccgacttcgggatggctgccgacaagaacaagttccccggggactcagtggtcactggtcgcggaagaatcaatggccggctcgtctacgtgttctcacaagactttactgtgttcggcggctccctgtcgggagcccacgcgcaaaagatctgcaagattatggatcaggccatcactgtgggagcgcctgtgattggactcaacgactccgggggagcaagaatccaggaaggagtggaaagccttgccggctacgctgacatcttcctccggaacgtgaccgcctctggagtgattccgcaaatctccctgatcatgggaccatgtgccgggggcgccgtgtactccccggcgctgactgacttcactttcatggtcaaggacacatcctacctgttcatcaccggtcccgacgtcgtgaagtccgtgaccaacgaggatgtgacccaggaagaactggggggggccaagacgcataccaccatgtcgggagtggcccaccgggccttcgagaacgatgtggacgccttgtgcaaccttcgggacttcttcaattatctcccgctgagcagccaggatccggccccagtgcgggaatgccacgacccttcggatcggttggtgcctgagctggataccatcgtgcccctcgaatccaccaaggcttacaacatggtcgacatcattcactccgtggtggacgagagggaattcttcgagattatgccgaactacgccaagaacatcattgtcggattcgcccgcatgaacggtcgaactgtgggcattgtcggaaaccagcctaaagtggcctccggttgcctggacatcaactcaagcgtgaagggtgccagatttgtgcggttttgtgacgcgttcaatattccgctgatcaccttcgtcgacgtcccgggcttcctgcctgggaccgcccaggaatacggcggcatcatcagacacggcgcgaagctcctctacgcgttcgcggaagccaccgtgcccaaggtcaccgtgatcactcgcaaggcatacggcggcgcatacgatgtgatgtcctccaagcacctgtgtggcgacaccaactacgcctggcccaccgccgagatcgccgtgatgggtgccaagggtgctgtcgagatcatcttcaagggacatgaaaacgtggaagctgcccaggccgagtacattgaaaagttcgctaaccccttccctgccgccgtgcggggatttgtggatgacattatccagccgagctcgaccagggccagaatctgctgcgatcttgatgtgttggccagcaaaaaggtccagcggccctggcggaaacacgccaacattccactg

SEQ ID NO: 9 - DNA, 1617 bp, synthetic construct
  misc_feature 1..1617: note = Description of Artificial Sequence: PCCB Coding Sequence - Codon-Optimized
  source 1..1617: mol_type = other DNA
atggccgcggcgcttagagtggccgctgtgggagccaggctgagcgtgctggccagcggtctgcgcgccgcagtgcgctcgctgtgtagccaggctacctccgtgaatgagcggatcgaaaacaagcggcgcaccgccctgttgggcggcggacagcggcgaattgacgcccaacacaagcggggaaagctcactgcgagggaaagaatctcactgctgctcgaccccgggtcgttcgtggaatcggatatgtttgtcgaacatagatgcgcagatttcggaatggccgctgacaagaacaagttcccgggagattccgtcgtgaccggaagggggcgcattaacgggagacttgtgtacgtgttcagccaggatttcacggtgttcggcggatcactgagcggtgcacatgcacagaagatctgcaagatcatggaccaggccattaccgtcggggcacctgtgatcggcctgaatgattcgggcggagcccggattcaagagggcgtggagtcactcgcgggttacgccgacattttcctgcggaacgtcaccgcctccggcgtgatccctcaaatcagcctcattatgggcccctgcgcgggcggtgccgtctactcacccgctctgaccgattttaccttcatggtcaaggacacctcctatctgtttatcactggaccagatgtggtcaagtccgtgaccaacgaggacgtcactcaggaagaactcggtggagcaaagacccacactactatgtccggggtcgcgcatagagctttcgaaaacgacgtcgatgctctctgtaacctgagggatttcttcaactaccttccactgtcgtcgcaagacccagcccccgtgcgcgagtgccacgatccctccgaccgcctggtgccggaactcgacactattgtccctctggagtcaaccaaggcctacaacatggtggacatcatccatagcgtcgtggatgaacgggagttcttcgaaatcatgcccaactatgcgaaaaatatcatcgtgggctttgcgcggatgaacggccgcaccgtgggcatagtgggcaaccagccgaaggtcgcgtcgggatgcctcgatatcaacagctctgtgaagggagcgcggttcgtgcgcttctgcgacgccttcaacatccccttgatcaccttcgtggatgtgcctgggttcttgcctggaaccgcccaggaatacggggggatcattcggcacggagcaaaactgctgtacgccttcgccgaggccactgtgccgaaagtgacagtgattacccggaaggcctacgggggtgcctacgacgtgatgagctccaagcacctgtgcggagacaccaattacgcgtggcctactgctgaaattgctgtcatgggagccaagggcgccgtggaaatcattttcaagggccacgaaaacgtcgaggccgcccaagctgagtacatcgagaagtttgccaacccgtttcctgcggctgtgcgcggcttcgtcgacgatatcattcagccctcgtccactcgcgcccgcatttgttgtgacctcgacgtgctggcgtccaagaaagtgcaaagaccgtggagaaagcatgcaaacatcccgctc

SEQ ID NO: 10 - DNA, 1617 bp, synthetic construct
  misc_feature 1..1617: note = Description of Artificial Sequence: PCCB Coding Sequence - Codon-Optimized
  source 1..1617: mol_type = other DNA
atggctgctgctctgagggtggctgctgtgggggctaggctgtctgtgctggcttctgggctgagggctgctgtgaggtctctgtgttctcaggctacatctgtgaatgagaggattgagaataagaggaggacagctctgctggggggggggcagaggaggattgatgctcagcataagagggggaagctgacagctagggagaggatttctctgctgctggatcctgggtcttttgtggagtctgatatgtttgtggagcataggtgtgctgattttgggatggctgctgataagaataagtttcctggggattctgtggtgacagggagggggaggattaatgggaggctggtgtatgtgttttctcaggattttacagtgtttggggggtctctgtctggggctcatgctcagaagatttgtaagattatggatcaggctattacagtgggggctcctgtgattgggctgaatgattctgggggggctaggattcaggagggggtggagtctctggctgggtatgctgatatttttctgaggaatgtgacagcttctggggtgattcctcagatttctctgattatggggccttgtgctgggggggctgtgtattctcctgctctgacagattttacatttatggtgaaggatacatcttatctgtttattacagggcctgatgtggtgaagtctgtgacaaatgaggatgtgacacaggaggagctggggggggctaagacacatacaacaatgtctggggtggctcatagggcttttgagaatgatgtggatgctctgtgtaatctgagggatttttttaattatctgcctctgtcttctcaggatcctgctcctgtgagggagtgtcatgatccttctgataggctggtgcctgagctggatacaattgtgcctctggagtctacaaaggcttataatatggtggatattattcattctgtggtggatgagagggagttttttgagattatgcctaattatgctaagaatattattgtggggtttgctaggatgaatgggaggacagtggggattgtggggaatcagcctaaggtggcttctgggtgtctggatattaattcttctgtgaagggggctaggtttgtgaggttttgtgatgcttttaatattcctctgattacatttgtggatgtgcctgggtttctgcctgggacagctcaggagtatggggggattattaggcatggggctaagctgctgtatgcttttgctgaggctacagtgcctaaggtgacagtgattacaaggaaggcttatgggggggcttatgatgtgatgtcttctaagcatctgtgtggggatacaaattatgcttggcctacagctgagattgctgtgatgggggctaagggggctgtggagattatttttaaggggcatgagaatgtggaggctgctcaggctgagtatattgagaagtttgctaatccttttcctgctgctgtgagggggtttgtggatgatattattcagccttcttctacaagggctaggatttgttgtgatctggatgtgctggcttctaagaaggtgcagaggccttggaggaagcatgctaatattcctctg

SEQ ID NO: 11 - DNA, 1617 bp, synthetic construct
  misc_feature 1..1617: note = Description of Artificial Sequence: PCCB Coding Sequence - Codon-Optimized
  source 1..1617: mol_type = other DNA
atggcagcagcactgagggtggcagcagtgggggcaaggctgtcagtgctggcatcagggctgagggcagcagtgaggtcactgtgttcacaggcaacatcagtgaatgagaggattgagaataagaggaggacagcactgctggggggggggcagaggaggattgatgcacagcataagagggggaagctgacagcaagggagaggatttcactgctgctggatccagggtcatttgtggagtcagatatgtttgtggagcataggtgtgcagattttgggatggcagcagataagaataagtttccaggggattcagtggtgacagggagggggaggattaatgggaggctggtgtatgtgttttcacaggattttacagtgtttggggggtcactgtcaggggcacatgcacagaagatttgtaagattatggatcaggcaattacagtgggggcaccagtgattgggctgaatgattcagggggggcaaggattcaggagggggtggagtcactggcagggtatgcagatatttttctgaggaatgtgacagcatcaggggtgattccacagatttcactgattatggggccatgtgcagggggggcagtgtattcaccagcactgacagattttacatttatggtgaaggatacatcatatctgtttattacagggccagatgtggtgaagtcagtgacaaatgaggatgtgacacaggaggagctggggggggcaaagacacatacaacaatgtcaggggtggcacatagggcatttgagaatgatgtggatgcactgtgtaatctgagggatttttttaattatctgccactgtcatcacaggatccagcaccagtgagggagtgtcatgatccatcagataggctggtgccagagctggatacaattgtgccactggagtcaacaaaggcatataatatggtggatattattcattcagtggtggatgagagggagttttttgagattatgccaaattatgcaaagaatattattgtggggtttgcaaggatgaatgggaggacagtggggattgtggggaatcagccaaaggtggcatcagggtgtctggatattaattcatcagtgaagggggcaaggtttgtgaggttttgtgatgcatttaatattccactgattacatttgtggatgtgccagggtttctgccagggacagcacaggagtatggggggattattaggcatggggcaaagctgctgtatgcatttgcagaggcaacagtgccaaaggtgacagtgattacaaggaaggcatatgggggggcatatgatgtgatgtcatcaaagcatctgtgtggggatacaaattatgcatggccaacagcagagattgcagtgatgggggcaaagggggcagtggagattatttttaaggggcatgagaatgtggaggcagcacaggcagagtatattgagaagtttgcaaatccatttccagcagcagtgagggggtttgtggatgatattattcagccatcatcaacaagggcaaggatttgttgtgatctggatgtgctggcatcaaagaaggtgcagaggccatggaggaagcatgcaaatattccactg

SEQ ID NO: 12 - DNA, 1617 bp, synthetic construct
  misc_feature 1..1617: note = Description of Artificial Sequence: PCCB Coding Sequence - Codon-Optimized
  source 1..1617: mol_type = other DNA
atggctgcagcactaagggtagcagcagtaggtgcaaggctatcagtactagcatcaggtctaagggcagcagtaaggtcactatgttcacaagcaacatcagtaaatgaaaggatagaaaataagaggaggacagcactactaggtggtgggcagaggaggattgatgctcagcataagagggggaagctgacagctagggagaggatttctctgctgctggatcctgggtcttttgtggagtctgatatgtttgtggagcataggtgtgctgattttgggatggctgctgataagaataagtttcctggggattctgtggtgacagggagggggaggattaatgggaggctggtgtatgtgttttctcaggattttacagtgtttggggggtctctgtctggggctcatgctcagaagatttgtaagattatggatcaggctattacagtgggggctcctgtgattgggctgaatgattctgggggggctaggattcaggagggggtggagtctctggctgggtatgctgatatttttctgaggaatgtgacagcttctggggtgattcctcagatttctctgattatggggccttgtgctgggggggctgtgtattctcctgctctgacagattttacatttatggtgaaggatacatcttatctgtttattacagggcctgatgtggtgaagtctgtgacaaatgaggatgtgacacaggaggagctggggggggctaagacacatacaacaatgtctggggtggctcatagggcttttgagaatgatgtggatgctctgtgtaatctgagggatttttttaattatctgcctctgtcttctcaggatcctgctcctgtgagggagtgtcatgatccttctgataggctggtgcctgagctggatacaattgtgcctctggagtctacaaaggcttataatatggtggatattattcattctgtggtggatgagagggagttttttgagattatgcctaattatgctaagaatattattgtggggtttgctaggatgaatgggaggacagtggggattgtggggaatcagcctaaggtggcttctgggtgtctggatattaattcttctgtgaagggggctaggtttgtgaggttttgtgatgcttttaatattcctctgattacatttgtggatgtgcctgggtttctgcctgggacagctcaggagtatggggggattattaggcatggggctaagctgctgtatgcttttgctgaggctacagtgcctaaggtgacagtgattacaaggaaggcttatgggggggcttatgatgtgatgtcttctaagcatctgtgtggggatacaaattatgcttggcctacagctgagattgctgtgatgggggctaagggggctgtggagattatttttaaggggcatgagaatgtggaggctgctcaggctgagtatattgagaagtttgctaatccttttcctgctgctgtgagggggtttgtggatgatattattcagccttcttctacaagggctaggatttgttgtgatctggatgtgctggcttctaagaaggtgcagaggccttggaggaagcatgctaatattcctctg

SEQ ID NO: 13 - DNA, 2211 bp, synthetic construct
  misc_feature 1..2211: note = Description of Artificial Sequence: AAV9 Nucleic Acid Sequence
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 14 - protein, 736 aa, synthetic construct
  REGION 1..736: note = Description of Artificial Sequence: AAV9 Amino Acid Sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 15 - DNA, 145 bp, synthetic construct
  misc_feature 1..145: note = Description of Artificial Sequence: AAV2 ITR
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 16 - protein, 728 aa, Homo sapiens
MAGFWVGTAPLVAAGRRGRWPPQQLMLSAALRTLKHVLYYSRQCLMVSRNLGSVGYDPNEKTFDKILVANRGEIACRVIRTCKKMGIKTVAIHSDVDASSVHVKMADEAVCVGPAPTSKSYLNMDAIMEAIKKTRAQAVHPGYGFLSENKEFARCLAAEDVVFIGPDTHAIQAMGDKIESKLLAKKAEVNTIPGFDGVVKDAEEAVRIAREIGYPVMIKASAGGGGKGMRIAWDDEETRDGFRLSSQEAASSFGDDRLLIEKFIDNPRHIEIQVLGDKHGNALWLNERECSIQRRNQKVVEEAPSIFLDAETRRAMGEQAVALARAVKYSSAGTVEFLVDSKKNFYFLEMNTRLQVEHPVTECITGLDLVQEMIRVAKGYPLRHKQADIRINGWAVECRVYAEDPYKSFGLPSIGRLSQYQEPLHLPGVRVDSGIQPGSDISIYYDPMISKLITYGSDRTEALKRMADALDNYVIRGVTHNIALLREVIINSRFVKGDISTKFLSDVYPDGFKGHMLTKSEKNQLLAIASSLFVAFQLRAQHFQENSRMPVIKPDIANWELSVKLHDKVHTVVASNNGSVFSVEVDGSKLNVTSTWNLASPLLSVSVDGTQRTVQCLSREAGGNMSIQFLGTVYKVNILTRLAAELNKFMLEKVTEDTSSVLRSPMPGVVVAVSVKPGDAVAEGQEICVIEAMKMQNSMTAGKTGTVKSVHCQAGDTVGEGDLLVELE

SEQ ID NO: 17 - protein, 539 aa, Homo sapiens
MAAALRVAAVGARLSVLASGLRAAVRSLCSQATSVNERIENKRRTALLGGGQRRIDAQHKRGKLTARERISLLLDPGSFVESDMFVEHRCADFGMAADKNKFPGDSVVTGRGRINGRLVYVFSQDFTVFGGSLSGAHAQKICKIMDQAITVGAPVIGLNDSGGARIQEGVESLAGYADIFLRNVTASGVIPQISLIMGPCAGGAVYSPALTDFTFMVKDTSYLFITGPDVVKSVTNEDVTQEELGGAKTHTTMSGVAHRAFENDVDALCNLRDFFNYLPLSSQDPAPVRECHDPSDRLVPELDTIVPLESTKAYNMVDIIHSVVDEREFFEIMPNYAKNIIVGFARMNGRTVGIVGNQPKVASGCLDINSSVKGARFVRFCDAFNIPLITFVDVPGFLPGTAQEYGGIIRHGAKLLYAFAEATVPKVTVITRKAYGGAYDVMSSKHLCGDTNYAWPTAEIAVMGAKGAVEIIFKGHENVEAAQAEYIEKFANPFPAAVRGFVDDIIQPSSTRARICCDLDVLASKKVQRPWRKHANIPL

SEQ ID NO: 18 - DNA, 278 bp, synthetic construct
  misc_feature 1..278: note = Description of Artificial Sequence: CBA Promoter
  source 1..278: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 19 - DNA, 304 bp, synthetic construct
  misc_feature 1..304: note = Description of Artificial Sequence: CMV Enhancer
  source 1..304: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 20 - DNA, 95 bp, synthetic construct
  misc_feature 1..95: note = Description of Artificial Sequence: SV40 Small T Intron
  source 1..95: mol_type = other DNA
gctctaaggtaaatataaaatttttaagtgtataatgtgttaaactactgattctaattgtttctctcttttagattccaacctttggaactgat

SEQ ID NO: 21 - DNA, 315 bp, synthetic construct
  misc_feature 1..315: note = Description of Artificial Sequence: rHBB Intron
  source 1..315: mol_type = other DNA
gtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctcaagagcctctgctaaccatgttcatgccttcttctttttcctacagctcctgggcaacgtgctggttattgtgctgtctcatcattttggcaaagaatt

SEQ ID NO: 22 - DNA, 215 bp, synthetic construct
  misc_feature 1..215: note = Description of Artificial Sequence: BGH polyadenylation signal sequence
  source 1..215: mol_type = other DNA
gcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 23 - DNA, 198 bp, synthetic construct
  misc_feature 1..198: note = Description of Artificial Sequence: SV40 Late polyadenylation signal sequence
  source 1..198: mol_type = other DNA
gatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttag

SEQ ID NO: 24 - skipped

SEQ ID NO: 25 - protein, 728 aa, Homo sapiens; identical to SEQ ID NO: 16

SEQ ID NO: 26 - protein, 702 aa, Homo sapiens
MAGFWVGTAPLVAAGRRGRWPPQQLMLSAALRTLKTFDKILVANRGEIACRVIRTCKKMGIKTVAIHSDVDASSVHVKMADEAVCVGPAPTSKSYLNMDAIMEAIKKTRAQAVHPGYGFLSENKEFARCLAAEDVVFIGPDTHAIQAMGDKIESKLLAKKAEVNTIPGFDGVVKDAEEAVRIAREIGYPVMIKASAGGGGKGMRIAWDDEETRDGFRLSSQEAASSFGDDRLLIEKFIDNPRHIEIQVLGDKHGNALWLNERECSIQRRNQKVVEEAPSIFLDAETRRAMGEQAVALARAVKYSSAGTVEFLVDSKKNFYFLEMNTRLQVEHPVTECITGLDLVQEMIRVAKGYPLRHKQADIRINGWAVECRVYAEDPYKSFGLPSIGRLSQYQEPLHLPGVRVDSGIQPGSDISIYYDPMISKLITYGSDRTEALKRMADALDNYVIRGVTHNIALLREVIINSRFVKGDISTKFLSDVYPDGFKGHMLTKSEKNQLLAIASSLFVAFQLRAQHFQENSRMPVIKPDIANWELSVKLHDKVHTVVASNNGSVFSVEVDGSKLNVTSTWNLASPLLSVSVDGTQRTVQCLSREAGGNMSIQFLGTVYKVNILTRLAAELNKFMLEKVTEDTSSVLRSPMPGVVVAVSVKPGDAVAEGQEICVIEAMKMQNSMTAGKTGTVKSVHCQAGDTVGEGDLLVELE

SEQ ID NO: 27 - protein, 681 aa, Homo sapiens
MAGFWVGTAPLVAAGRRGRWPPQQLMLSAALRTLKHVLYYSRQCLMVSRNLGSVGYDPNEKTFDKILVANRGEIACRVIRTCKKMGIKTVAIHSDVDASSVHVKMADEAVCVGPAPTSKSYLNMDAIMEAIKKTRAQAVHPGYGFLSENKEFARCLAAEDVVFIGPDTHAIQAMGDKIESKLLAKKAEVNTIPGFDGVVKDAEEAVRIAREIGYPVMIKASAGGGGKGMRIAWDDEETRDGFRLSSQEAASSFGDDRLLIEKFIDNPRHIEIQVLGDKHGNALWLNERECSIQRRNQKVVEEAPSIFLDAETRRAMGEQAVALARAVKYSSAGTVEFLVDSKKNFYFLEMNTRLQVEHPVTECITGLDLVQEMIRVAKGYPLRHKQADIRINGWAVECRVYAEDPYKSFGLPSIGRLSQYQEPLHLPGVRVDSGIQPGSDISIYYDPMISKLITYGSDRTEALKRMADALDNYVIRGVTHNIALLREVIINSRFVKGDISTKFLSDVYPDGFKGHMLTKSEKNQLLAIASSLFVAFQLRAQHFQENSRMPVIKPDIANWELSVKLHDKVHTVVASNNGSVFSVEVDGSKLNVTSTWNLASPLLSVSVDGTQRTVQCLSREAGGNMSIQFLGTVVAEGQEICVIEAMKMQNSMTAGKTGTVKSVHCQAGDTVGEGDLLVELE

SEQ ID NO: 28 - protein, 539 aa, Homo sapiens; identical to SEQ ID NO: 17

SEQ ID NO: 29 - protein, 559 aa, Homo sapiens
MAAALRVAAVGARLSVLASGLRAAVRSLCSQATSVNERIENKRRTALLGGGQRRIDAQHKRGKLTARERISLLLDPGSFVESDMFVEHRCADFGMAADKNKFPGDSVVTGRGRINGRLVYVFSQQIIGWAQWLPLVISALWEAEDFTVFGGSLSGAHAQKICKIMDQAITVGAPVIGLNDSGGARIQEGVESLAGYADIFLRNVTASGVIPQISLIMGPCAGGAVYSPALTDFTFMVKDTSYLFITGPDVVKSVTNEDVTQEELGGAKTHTTMSGVAHRAFENDVDALCNLRDFFNYLPLSSQDPAPVRECHDPSDRLVPELDTIVPLESTKAYNMVDIIHSVVDEREFFEIMPNYAKNIIVGFARMNGRTVGIVGNQPKVASGCLDINSSVKGARFVRFCDAFNIPLITFVDVPGFLPGTAQEYGGIIRHGAKLLYAFAEATVPKVTVITRKAYGGAYDVMSSKHLCGDTNYAWPTAEIAVMGAKGAVEIIFKGHENVEAAQAEYIEKFANPFPAAVRGFVDDIIQPSSTRARICCDLDVLASKKVQRPWRKHANIPL

SEQ ID NO: 30 - DNA, 1412 bp, Homo sapiens
  source 1..1412: mol_type = genomic DNA
tctcaaggtgactctgcaggagatgcaaagtagttaagaggagaacccccaatactgatgtgtttataggtgaggtggtctgatgtcttagatttgctttaaaatattcctgcacaaaataaaaaggggaatagatgaaacaagattgacaaaatgttgattcattgacgcggtgtaatgggtatctgggtgatcatagtacaattttatctcttttgtatatattttaaaatttccataatatagcaaaacatgagcccagtccctcaaggcagccagatttgggttcaaaccctagtttgaccttttactcgctgagtgactttaccctgctacagcttcagtttctccaaatggaaataatatatactccattaaggttcttgtaaactctcaagtaagagggaatctgaggctctagcacacggcccggcccttaatagttgtactaatttttattttatttttcgaaacggagtttgctctgtcacccaggctggagtgtagtggcatgatccggctcactgcaacctccacctccagggttcaagcaattctcctgcctcagcctcctcccgagtaactgggattacaggagtgcgccaccacgcccagctaatttttgtattttttagtagagacggggtttcggcatgttgtccaggctggtctccaactcctgacctcaggttatcctcccgcctcggcctcccagagtgctgggattacaggcgtgagccactgcgcctggccagtagttgtactaacaagggtttctgcttgcgagcaacaaagactgactctagttgtggctaatttctttcacacacacatattaagtgccagaggtaggacttgagctcggatctctgatttccagacctagaactttttatgtgttattacagtcgttaaaaaccttacctggatttgtgctttgcaatttacgctttcgaacatgcaggaatgtagtttgggcatttgtctggaacttcggattctcgggactccagggtggccaaggcgcggaggtgactcaatgacattgaccggcctggatgcacgtgggttgctgagccgatgctgggaaggtacggggtttgcccggtgcaggccgccggaactcacgcacgcccctgtctctctcactcctggggtcggctctggccggtggcctccagctcagctagcggccggacgcgcagcccgccgactagccctccaggtcctagcgcgactcccggatcccggatccggcggatctggccgaccactcaccctcccctttccgcaagttagggggcggggcatcgggtttctggctcgtgatttgccggagctcctgcgctccccttctccaccccctccggctgtgtgagaggtcagcagaggggcggtctgcggggacaaca

SEQ ID NO: 31 - DNA, 275 bp, Homo sapiens
  source 1..275: mol_type = genomic DNA
aggatttataacctttcagtcatcacccaatttaattagccatttgcatgatgctttcacacacaattgattcaagcattatacaggaacacccctgtgcagctacgtttacgtcgtcatttattccacagagtcaagaccaatattctgccaaaaaatcaccaatggaaattttcattgatataaatacttgtacatatgatttgtacttctgctgtgagattccctagtgtcaaaattaaatcaataaaactgagcatttgtctaaatattag

SEQ ID NO: 32 - DNA, 1378 bp, Homo sapiens
  source 1..1378: mol_type = genomic DNA
gctggccccagccccagagtttctgatttaaaggtctggagtaagttctgagaattcgagcttgcatttccaactcactcccaggtgatactgaagttgctggattgacaagattccctccttgatcaaacattggtcaggctcttctgagctctctctttgactaggcctgagtgtgggctcccatgtcattccttgtggaattccattttagcaagaaacttgccaagtcagtttagccagaatcctccattcttgatatctgattactctccatatctggttttgacccataccgccaccatcccccaggtgatgttcagttaccctgacctgccttgggcaggaatcctgttaggtcaatttagccagattccccgccaatgtttcctcttagtaattttccatccactgacttccacacactgccccttagctataaattaccactttgctatattcagcccaatcgctctgacttatggcaaaactctatccctacctctgtcagattgggcctgaataaagtttgccttaccattcttaaacatgaataattaagtgtcatgaatagttttttctttaatgggatccatactttgagaactactgattagaggttaagggtcctgtgctcattatctggcacatattaatgataggtctctgagtttgaatcctggccaaccacattgtcagtcttggcttttttttttttttttttttgagacagggttttgcccaggctggagtgcagtgatgcaatcacggctcactgcagcctcgacttcctgggccaagccatcctgccgccttagcctcccgagtagctggggtcacaggtgcaagccgccatgcccagctaatttttaatttttttgtagaggtggggtcttactatattgctcaggctggtcttgggctcctgggctcacctacctcggcctcagtcttgcctcttgatcaaatcactcatctctctgtgcttgatttacctcatctgtgaaatccagatagtaagctagtgcatggtaagtgcaaaaagcaacacctggcacaatgcttaataaatgttggttaaatcttataatcattttctgtaggaagtgggacgacaaagttaagggtctaggtggtcagagaagagcaaggactatgtcccttttttgtgcagccaggcaggccctgtactttttctctgcgcgaccctcctcgggcagcaccgggtctcgaactctcacgccttactcccaagcaggttctgtagtccagcccccgcggctccctacagccagccgatgctgcgcaagcgccgtacccacgctttagcacatgcgtactcaggtgcgccggtaggggacgcgccggcacagcaaaa

SEQ ID NO: 33 - DNA, 159 bp, Homo sapiens
  source 1..159: mol_type = genomic DNA
acaaatcaaaggaaaagaaaccaagaactgaattactgtctgcccattcacatcccattcctgccttttgcaatcatgaaacctgggaatccaaatagttggataacttagaataactaagtttattaaattctagaaagatctcttttgtgccttact

SEQ ID NO: 34 - DNA, 793 bp, Homo sapiens
  source 1..793: mol_type = genomic DNA
tttttgtattttttagtagagacggggtttcggcatgttgtccaggctggtctccaactcctgacctcaggttatcctcccgcctcggcctcccagagtgctgggattacaggcgtgagccactgcgcctggccagtagttgtactaacaagggtttctgcttgcgagcaacaaagactgactctagttgtggctaatttctttcacacacacatattaagtgccagaggtaggacttgagctcggatctctgatttccagacctagaactttttatgtgttattacagtcgttaaaaaccttacctggatttgtgctttgcaatttacgctttcgaacatgcaggaatgtagtttgggcatttgtctggaacttcggattctcgggactccagggtggccaaggcgcggaggtgactcaatgacattgaccggcctggatgcacgtgggttgctgagccgatgctgggaaggtacggggtttgcccggtgcaggccgccggaactcacgcacgcccctgtctctctcactcctggggtcggctctggccggtggcctccagctcagctagcggccggacgcgcagcccgccgactagccctccaggtcctagcgcgactcccggatcccggatccggcggatctggccgaccactcaccctcccctttccgcaagttagggggcggggcatcgggtttctggctcgtgatttgccggagctcctgcgctccccttctccaccccctccggctgtgtgagaggtcagcagaggggcggtctgcggggacaaca

SEQ ID NO: 35 - DNA, 168 bp, Homo sapiens
  source 1..168: mol_type = genomic DNA
aggatttataacctttcagtcatcacccaatttaattagccatttgcatgatgctttcacacacaattgattcaagcattatacaggaacacccctgtgcagctacgtttacgtcgtcatttattccacagagtcaagaccaatattctgccaaaaaatcaccaatgg

SEQ ID NO: 36 - DNA, 468 bp, Homo sapiens
  source 1..468: mol_type = genomic DNA
gctggtcttgggctcctgggctcacctacctcggcctcagtcttgcctcttgatcaaatcactcatctctctgtgcttgatttacctcatctgtgaaatccagatagtaagctagtgcatggtaagtgcaaaaagcaacacctggcacaatgcttaataaatgttggttaaatcttataatcattttctgtaggaagtgggacgacaaagttaagggtctaggtggtcagagaagagcaaggactatgtcccttttttgtgcagccaggcaggccctgtactttttctctgcgcgaccctcctcgggcagcaccgggtctcgaactctcacgccttactcccaagcaggttctgtagtccagcccccgcggctccctacagccagccgatgctgcgcaagcgccgtacccacgctttagcacatgcgtactcaggtgcgccggtaggggacgcgccggcacagcaaaa

SEQ ID NO: 37 - DNA, 102 bp, Homo sapiens
  source 1..102: mol_type = genomic DNA
acaaatcaaaggaaaagaaaccaagaactgaattactgtctgcccattcacatcccattcctgccttttgcaatcatgaaacctgggaatccaaatagttgg

SEQ ID NO: 38 - DNA, 3360 bp, synthetic construct
  misc_feature 1..3360: note = Description of Artificial Sequence: rAAV with native PCCA cDNA sequence (DTC346)
  source 1..3360: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgcgtcgaggtgagccccacgttctgcttcactctccccatctcccccccctccccacccccaattttgtatttatttattttttaattattttatgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggctctaaggtaaatataaaatttttaagtgtataatgtgttaaactactgattctaattgtttctctcttttagattccaacctttggaactgatgccgccaccatggcggggttctgggtcgggacagcaccgctggtcgctgccggacggcgtgggcggtggccgccgcagcagctgatgctgagcgcggcgctgcggaccctgaagcatgttctgtactattcaagacagtgcttaatggtgtcccgtaatcttggttcagtgggatatgatcctaatgaaaaaacttttgataaaattcttgttgctaatagaggagaaattgcatgtcgggttattagaacttgcaagaagatgggcattaagacagttgccatccacagtgatgttgatgctagttctgttcatgtgaaaatggcggatgaggctgtctgtgttggcccagctcccaccagtaaaagctacctcaacatggatgccatcatggaagccattaagaaaaccagggcccaagctgtacatccaggttatggattcctttcagaaaacaaagaatttgccagatgtttggcagcagaagatgtcgttttcattggacctgacacacatgctattcaagccatgggcgacaagattgaaagcaaattattagctaagaaagcagaggttaatacaatccctggctttgatggagtagtcaaggatgcagaagaagctgtcagaattgcaagggaaattggctaccctgtcatgatcaaggcctcagcaggtggtggtgggaaaggcatgcgcattgcttgggatgatgaagagaccagggatggttttagattgtcatctcaagaagctgcttctagttttggcgatgatagactactaatagaaaaatttattgataatcctcgtcatatagaaatccaggttctaggtgataaacatgggaatgctttatggcttaatgaaagagagtgctcaattcagagaagaaatcagaaggtggtggaggaagcaccaagcatttttttggatgcggagactcgaagagcgatgggagaacaagctgtagctcttgccagagcagtaaaatattcctctgctgggaccgtggagttccttgtggactctaagaagaatttttatttcttggaaatgaatacaagactccaggttgagcatcctgtcacagaatgcattactggcctggacctagtccaggaaatgatccgtgttgctaagggctaccctctcaggcacaaacaagctgatattcgcatcaacggctgggcagttgaatgtcgggtttatgctgaggacccctacaagtcttttggtttaccatctattgggagattgtctcagtaccaagaaccgttacatctacctggtgtccgagtggacagtggcatccaaccaggaagtgatattagcatttattatgatcctatgatttcaaaactaatcacatatggctctgatagaactgaggcactgaagagaatggcagatgcactggataactatgttattcgaggtgttacacataatattgcattacttcgagaggtgataatcaactcacgctttgtaaaaggagacatcagcactaaatttctctccgatgtgtatcctgatggcttcaaaggacacatgctaaccaagagtgagaagaaccagttattggcaatagcatcatcattgtttgtggcattccagttaagagcacaacattttcaagaaaattcaagaatgcctgttattaaaccagacatagccaactgggagctctcagtaaaattgcatgataaagttcataccgtagtagcatcaaacaatgggtcagtgttctcggtggaagttgatgggtcgaaactaaatgtgaccagcacgtggaacctggcttcgcccttattgtctgtcagcgttgatggcactcagaggactgtccagtgtctttctcgagaagcaggtggaaacatgagcattcagtttcttggtacagtgtacaaggtgaatatcttaaccagacttgccgcagaattgaacaaatttatgctggaaaaagtgactgaggacacaagcagtgttctgcgttccccgatgcccggagtggtggtggccgtctctgtcaagcctggagacgcggtagcagaaggtcaagaaatttgtgtgattgaagccatgaaaatgcagaatagtatgacagctgggaaaactggcacggtgaaatctgtgcactgtcaagctggagacacagttggagaaggggatctgctcgtggagctggaatgaatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 39 - DNA, 2806 bp, synthetic construct
  misc_feature 1..2806: note = Description of Artificial Sequence: rAAV with native PCCB cDNA sequence
  source 1..2806: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggctctaaggtaaatataaaatttttaagtgtataatgtgttaaactactgattctaattgtttctctcttttagattccaacctttggaactgatctcgaggccgccaccatggcggcggcattacgggtggcggcggtcggggcaaggctcagcgttctggcgagcggtctccgcgccgcggtccgcagcctttgcagccaggccacctctgttaacgaacgcatcgaaaacaagcgccggaccgcgctgctgggagggggccaacgccgtattgacgcgcagcacaagcgaggaaagctaacagccagggagaggatcagtctcttgctggaccctggcagctttgttgagagcgacatgtttgtggaacacagatgtgcagattttggaatggctgctgataagaataagtttcctggagacagcgtggtcactggacgaggccgaatcaatggaagattggtttatgtcttcagtcaggattttacagtttttggaggcagtctgtcaggagcacatgcccaaaagatctgcaaaatcatggaccaggccataacggtgggggctccagtgattgggctgaatgactctgggggagcacggatccaagaaggagtggagtctttggctggctatgcagacatctttctgaggaatgttacggcatccggagtcatccctcagatttctctgatcatgggcccatgtgctggtggggccgtctactccccagccctaacagacttcacgttcatggtaaaggacacctcctacctgttcatcactggccctgatgttgtgaagtctgtcaccaatgaggatgttacccaggaggagctcggtggtgccaagacccacaccaccatgtcaggtgtggcccacagagcttttgaaaatgatgttgatgccttgtgtaatctccgggatttcttcaactacctgcccctgagcagtcaggacccggctcccgtccgtgagtgccacgatcccagtgaccgtctggttcctgagcttgacacaattgtccctttggaatcaaccaaagcctacaacatggtggacatcatacactctgttgttgatgagcgtgaattttttgagatcatgcccaattatgccaagaacatcattgttggttttgcaagaatgaatgggaggactgttggaattgttggcaaccaacctaaggtggcctcaggatgcttggatattaattcatctgtgaaaggggctcgttttgtcagattctgtgatgcattcaatattccactcatcacttttgttgatgtccctggctttctacctggcacagcacaggaatacgggggcatcatccggcatggtgccaagcttctctacgcatttgctgaggcaactgtacccaaagtcacagtcatcaccaggaaggcctatggaggtgcctatgatgtcatgagctctaagcacctttgtggtgataccaactatgcctggcccaccgcagagattgcagtcatgggagcaaagggcgctgtggagatcatcttcaaagggcatgagaatgtggaagctgctcaggcagagtacatcgagaagtttgccaaccctttccctgcagcagtgcgagggtttgtggatgacatcatccaaccttcttccacacgtgcccgaatctgctgtgacctggatgtcttggccagcaagaaggtacaacgtccttggagaaaacatgcaaatattccattgtaatctagagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa